(19) United States
(12) Patent Application Publication
(10) Pub. No.: US 2022/0187315 A1
(43) Pub. Date: Jun. 16, 2022

(54) METHODS AND COMPOSITIONS FOR DIAGNOSING DEPRESSION

(71) Applicants: Rima F. KADDURAH-DAOUK, Belmont, MA (US); John A. RUSH, Durham, NC (US); Siamk Mahmoudian DEHKORDI, Durham, NC (US); Sudeepa BHATTCHARYYA, Little Rock, AR (US); Ranga R. KRISHNAN, Chicago, IL (US); Mark A. FRYE, Rochester, MN (US); Ahmed T. AHMED, Rochester, MN (US); Richard M. WEINSHILBOUM, Rochester, MN (US); Boadie W. DUNLOP, Atlanta, GA (US); Wei JIA, Honolulu, HI (US); Duke University, Durham, NC (US); UNIVERSITY OF ARKANSAS FOR MEDICAL SCIENCES, Little Rock, AZ (US); RUSH MEDICAL COLLEGE, Chicago, IL (US); MAYO CLINIC, Rochester, MN (US); EMORY UNIVERSITY SCHOOL OF MEDICINE, Atlanta, GA (US); UNIVERSITY OF HAWAII CANCER CENTER, Honolulu, HI (US)

(72) Inventors: Rima F. Kaddurah-Daouk, Belmont, MA (US); Augustus John Rush, Jr., Durham, NC (US); Siamak Mahmoudian Dehkordi, Durham, NC (US); Sudeepa Bhattcharyya, Little Rock, AR (US); Ranga R. Krishnan, Chicago, IL (US); Mark A. Frye, Rochester, MN (US); Ahmed T. Ahmed, Rochester, MN (US); Richard M. Weinshilboum, Rochester, MN (US); Boadie W. Dunlop, Atlanta, GA (US); Wei Jia, Honolulu, HA (US)

(21) Appl. No.: 17/438,156

(22) PCT Filed: Mar. 6, 2020
(86) PCT No.: PCT/US2020/021433

§ 371 Date: Sep. 10, 2021

Related U.S. Application Data
(60) Provisional application No. 62/817,635, filed on Mar. 13, 2019.

(51) Int. Cl. G01N 33/68; G01N 33/92; G01N 33/94; A61P 25/24

(52) U.S. Cl. CPC G01N 33/6893; G01N 33/92; G01N 2800/304; A61P 25/24; G01N 33/942

(57) ABSTRACT

Described herein are methods and composition for diagnosing and evaluating the treatment of depression using one or more biomarker metabolites for the diagnosis and monitoring treatment efficacy. In one aspect, the biomarker metabolites can be used to screen subjects for the likelihood of developing depression, the diagnosis thereof, monitoring the efficacy of treatment, and evaluating a subject's propensity for responding to treatment.

### CROSS-REFERENCE TO RELATED APPLICATIONS

This application claims priority to U.S. Provisional Patent Application No. 62/817,635, filed on Mar. 13, 2019, which is incorporated by reference here in in its entirety.

### FEDERALLY SPONSORED RESEARCH

This invention was made with United States government support under National Institutes of Health/National Institute of Mental Health grant number MH108348. The United States government has certain rights in the invention.

### TECHNICAL FIELD

Described herein are methods and composition for diagnosing and evaluating the treatment of depression using one or more biomarker metabolites for the diagnosis and monitoring treatment efficacy. In one aspect, the biomarker metabolites can be used to screen subjects for the likelihood of developing depression, the diagnosis thereof, monitoring the efficacy of treatment, and evaluating a subject's propensity for responding to treatment.

### BACKGROUND

Major depressive disorder (MDD) is a common, often disabling condition affecting over 300 million individuals worldwide, but much about its pathobiology and the biology of treatment response is unknown. World Health Organization W. Depression and Other Common Mental Disorders: Global Health Estimates (World Health Organization, Geneva, 2017). There is a growing interest in the role of metabolic dysregulation in the pathogenesis of MDD, with small studies implicating alterations in several pathways, including neurotransmission (GABA, glutamine, tryptophan, phenylalanine), nitrogen metabolomics, methylation, and lipid metabolism (Paige et al., Int. J. Geriatr. Psychiatry 22, 418-423 (2007); Steffens et al., J. Geriatr. Psychiatry Neurol. 23, 138-146 (2010); Gadad et al., J. Alfective Dis. 233, 3-14, (2018); MacDonald et al., Am. J. Med. Gen. Part B, Neuropsychiatric Gen. 180: 122-137 (2019); Pedrini, et al., Prog. Neuro-psychopharmacol. Biol. Psychiatry 93, 182-188 (2019)). Patients achieving remission from MDD with an antidepressant appear to have a metabolic state distinct from that of never-depressed individuals, with alterations in methylation, purine metabolism and oxidative stress pathways (Kaddurah-Daouk et al., Sci. Rep. 2, 667 (2012)).

Selective serotonin reuptake inhibitors (SSRIs) are common first-line agents used for the treatment of MDD, yet their effect varies across patients (Crismon et al., J. Clin. Psychiatry 60, 142-156 (1999); Anderson et al., J. Psychopharm. 22, 343-396 (2008). Roughly 40% of patients do not respond, and more than two thirds fail to achieve remission of symptoms with SSRI treatment (Rush et al., N. Engl. J. Med. 354, 1231-1242 (2006)). Hence, in clinical practice a “trial and error” approach is used to find an effective, yet well-tolerated therapy (Rush et al., Am. J. Psychiatry 163, 1905-1917 (2006)). SSRIs are believed to work by increasing extracellular availability of the neurotransmitter serotonin by limiting its reuptake into presynaptic neurons. Other mechanisms are also thought to contribute to therapeutic benefit.

Metabolomics provides enabling tools to map global metabolic changes in neuropsychiatric diseases and the effects of treatment. Kaddurah-Daouk et al., Sci. Rep. 2, 667 (2012); Paige et al., Int. J. Geratr. Psych. 418-423 (2007); Steffens et al., J. Geriatr Psychiatry Neurol. 23, 138-146 (2010); Wenk, Nat. Rev. Drug Disc. 4, 594-610 (2005); Griffin, Philosop. Trans. Royal Soc. London. Series B, Biol. Sci. 361, 147-161 (2006); Kaddurah-Daouk and Krishnan, Neuropsychopharmacol. 34, 173-186 (2009); Kaddurah-Daouk and Weinshilboum, Clin Pharmacol. Ther. 98, 71-75 (2015); Kaddurah-Daouk and Weinshilboum, Clin Pharmacol. Ther. 95, 154-167 (2014); Beger et al., Metabolomics 12, 149-149 (2016). Pharmacometabolomics refers to the effects on the metabolome resulting from exposure to drugs. Early pharmacometabolomic studies in MDD found that sertraline produced changes in intermediates of the tricarboxylic acid and urea cycles, fatty acids, intermediates of lipid biosynthesis, and amino acids. Kaddurah-Daouk et al. Transl. Psychiatry 3, e223 (2013). Sertraline responders had higher pretreatment levels of 5-methoxytryptamine (5-MTPM), greater reduction in 5-MTPM levels after treatment, an increase in 5-methoxytryptophol and melatonin levels, and decreases in the kynurenine/melatonin ratio post-treatment compared to pretreatment, indicating that improvement from a depressed state correlated with a shift in utilization of tryptophan from kynurenine to melatonin and its related methoxy-indole pathway metabolites (Zhu et al., PLoS One 8, e68283 (2013).

A pharmacometabolomic study of intravenous ketamine, which induces a rapid antidepressant effect, detected (Rotroff et al., wTransl. Psychiatry 6, e894 (2016) changes in metabolites related to tryptophan metabolism (e.g., indole-3-acetate and methionine) acyl-carnitine, urea cycle (e.g., citrulline, arginine and omithine), and lipid metabolism during a 2 h infusion. Changes in phospholipids were associated with changes in depression severity during treatment. The use of metabolomics data can also inform genomics studies and together can identify novel genes and pathways implicated in variation of response to treatment.

Symptom features alone have not been successful as was to address the well-known biological and treatment-response heterogeneity in major depression. More recent efforts have used molecular, physiological, imaging, neuropsychological and clinical data to identify more homogeneous subgroupings of depression and to facilitate more personalized patient care.

We recently proposed 3 symptomatically defined phenotypes based on items from the 17 item Hamilton Rating Scale for Depression (HRSD˜) that may reflect somewhat distinct neural processes or circuits which could establish the framework for subsequent biomarker analyses. These phenotypes focused on core depressive symptoms (depressed mood #1, work and activities #7) [CD+] reflecting the positive and negative valence circuits; neuro-vegetative symptoms of melancholia [NVSM+] (late insomnia #6, somatic gastrointestinal #12) and anxious features reflecting the fear circuit using items #9 (agitation), #10 (psychological anxiety), #11 (somatic anxiety), and #15 (hypochondriasis) (ANX+). Our approach attempts to use the Research Domain Criteria (RDoC) research framework to establish the basis for depression by integrating clinical, psychological, neuro-functional, biological, behavioral, physiological perspectives.

There is a need for methods to diagnose, treat, differentiate depression subtypes, monitor efficacy, and predict treatment success using metabolomics.

### SUMMARY

One embodiment described herein is a method for diagnosing or detecting depression in a subject, the method comprising: obtaining a sample from a subject and determining concentration levels of one or more biomarker metabolites in the sample from the subject; and diagnosing the subject as having depression or an increased risk of depression when the concentration levels of the one or more biomarker metabolites in the sample from the subject is different from (greater or less) than concentration levels the one or more biomarker metabolites in a control sample.

In one aspect, the method further comprises: initially treating the subject for depression by administering an effective amount sufficient to attenuate, reduce, or eliminate the symptoms of depression of one or more of antidepressants, cognitive behavior therapy, exercise, dietary supplements, prebiotics, probiotics, dietary changes, or an elimination diet; obtaining a second sample from the subject and determining concentration levels of one or more biomarker metabolites in the sample from the subject; evaluating the concentration level of the one or more biomarker metabolites in comparison to control concentration levels of the one or more biomarker metabolites; evaluating the efficacy of the depression treatment; and continuing the one or more initial depression treatments; administering one or more additional depression treatment; or administering one or more second depression treatments (switching the treatment regimen).

In another aspect, the biomarker metabolite comprises one or more of: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine; Biogenic Amines: acetylomithine; asymmetric dimethylarginine; alpha-aminoadipic acid; carnosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline; Glycerophospholipids (i.e., PC aa): PC aa C24:0; PC aa C26:0; PC aa C28:1; PC aa C30:0; PC as C32:0; PC aa C32:1; PC as C32:3; PC aa C34:1; PC aa C34:2; PC aa C34:3; PC aa C34:4; PC aa C36:0; PC as C36:1; PC as C36:2; PC aa C36:3; PC aa C36:4; PC aa C36:5; PC aa C36:6; PC aa C38:0; PC aa C38:3; PC aa C38:4; PC aa C38:5; PC aa C38:6; PC aa C40:1; PC aa C40:2; PC aa C40:3; PC aa C40:4; PC aa C40:5; PC aa C40:6; PC as C42:0; PC aa C42:1; PC aa C42:2; PC aa C42:4; PC aa C42:5; or PC aa C42:6; Glycerophospholipids (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6; Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1; or combinations thereof.

In another aspect, the biomarker metabolite comprises one or more of: tryptophan (Trp), tyrosine (Tyr), phenylalanine (Phe), methionine (Met), cysteine (Cys), 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid homogentisic acid (4HPLA HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), or theophylline or combinations thereof. In another aspect, the biomarker metabolites comprise one or more of: carnitine, propionylcarnitine; butyrylcarnitine, isovalerylcarnitine, α-aminoadipic acid, glutamate and proline; isoleucine, valine, tryptophan, tyrosine, phenylalanine, methionine, methionine-sulfoxide, sarcosine; phosphatidylcholines, or sphingolipids.

In another aspect, the biomarker metabolite comprises one or more of: short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA)); 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; In another aspect, the biomarker metabolite concentration level is greater than the control concentration level. In another aspect, the biomarker metabolite concentration level is less than the control concentration level. In another aspect, two or more biomarker metabolite concentration levels covary and are greater than the control or covary and are less than the control concentration levels (positive correlation). In another aspect, two or more biomarker metabolite concentration levels vary dissimilarly (negative correlation) compared to the control concentration levels.

In another aspect, the sample from the subject is one or more of whole blood, serum, plasma, urine, saliva, or other body fluids. In another aspect, the control sample is from an untreated subject or a subject or population of subjects not experiencing depression or not at risk for depression.

In another aspect, the depression is Major Depression Disorder (MDD), core depression (CD+), anxious depression (ANX+), neurovegetative symptoms of melancholia (NVSM+), or subclinical characteristics associated with depression.

In another aspect, the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the efficacy of the depression treatment is evaluated using the Hamilton Depression Rating Scale (HRSD₁₇), the Quick Inventory of Depressive Symptomatology (QIDS), subscales thereof, or specific questions thereof.

Another embodiment described herein is a method of treating depression comprising: administering an effective amount sufficient to attenuate, reduce, or eliminate the symptoms of depression of an antidepressant or cognitive behavioral therapy or a combination thereof to a subject having depression or at risk of depression; obtaining a blood sample from the subject; and measuring concentration levels in the subject's blood sample of one or more biomarker metabolites comprising: short-chain acylcarnitines, medium-chain acylcarnitines, long-chain acylcarnitines, amino acids, biogenic amines, glycerophospholipids, sphingolipids, or combinations thereof.

In one aspect, the treatment is maintained or adjusted based on the concentration levels of one or more biomarker metabolites, ratios of biomarker metabolites, or combinations thereof. In another aspect, the depression is Major Depression Disorder (MDD), core depression (CD+), anxious depression (ANX+), neurovegetative symptoms of melancholia (NVSM+), or subclinical characteristics associated with depression. In another aspect, the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram.

In another aspect, the treatment further comprises administering one or more of exercise, dietary supplements, prebiotics, probiotics, probiotics, dietary changes, or an elimination diet.

Another embodiment described herein is a method of treating depression and evaluating the treatment efficacy comprising: identifying a subject suffering from or at risk of developing depression; administering an effective amount sufficient to attenuate, reduce, or eliminate the symptoms of depression of one or more antidepressants, cognitive behavior therapy, exercise, dietary supplements, prebiotics, probiotics, or dietary changes, or a combination thereof over a period of time; obtaining a sample from the subject and evaluating concentration levels of one or more biomarker metabolites; evaluating the concentration level of the one or more biomarker metabolites in comparison to control concentration levels of the one or more biomarker metabolites; evaluating the efficacy of the depression treatment; and continuing the administration one or more first depression treatments; administering an additional depression treatment; or administering one or more second depression treatments (switching the treatment regimen).

In one aspect, the biomarker metabolite comprises one or more of: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine; Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline; Glycerophospholipids (i.e., PC aa): PC as C24:0; PC as C26:0; PC aa C28:1; PC aa C30:0; PC as C32:0; PC aa C32:1; PC as C32:3; PC as C34:1; PC aa C34:2; PC as C34:3; PC aa C34:4; PC aa C36:0; PC as C36:1; PC aa C36:2; PC aa C36:3; PC aa C36:4; PC aa C36:5; PC as C36:6; PC aa C38:0; PC aa C38:3; PC as C38:4; PC aa C38:5; PC aa C38:6; PC aa C40:1; PC as C40:2; PC aa C40:3; PC as C40:4; PC as C40:5; PC aa C40:6; PC as C42:0; PC aa C42:1; PC aa C42:2; PC as C42:4; PC aa C42:5; or PC as C42:6; Glycerophospholipid ethers (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6; Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1; short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA)); 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; or combinations thereof.

In another aspect, the sample from the subject is one or more of whole blood, serum, plasma, urine, saliva, or other body fluids. In another aspect, the control sample is from an untreated subject or a subject or population of subjects not experiencing depression or not at risk for depression.

In another aspect, the biomarker metabolite concentration level is greater than the control concentration level. In another aspect, the biomarker metabolite concentration level is less than the control concentration level. In another aspect, two or more biomarker metabolite concentration levels covary and are greater than the contori or covary and are less than the control concentration levels (positive correlation). In another aspect, two or more biomarker metabolite concentration levels vary dissimilarly (negative correlation) compared to the control concentration levels.

In another aspect, the period of time is at least: 4-weeks, 8-weeks, 16-weeks, 32-weeks, 64-weeks, or greater than 64-weeks. In another aspect, the depression is Major Depression Disorder (MDD), core depression (CD+), anxious depression (ANX+), neurovegetative symptoms of melancholia (NVSM+), or subclinical characteristics associated with depression. In another aspect, the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram. In another aspect, the efficacy of the depression treatment is evaluated using the Hamilton Depression Rating Scale (HRSD17), the Quick Inventory of Depressive Symptomatology (QIDS), subscales thereof, or specific questions thereof.

Another embodiment described herein is a method of evaluating the efficacy of depression treatment, the method comprising: identifying a subject suffering from or at risk of developing depression; obtaining a sample from the subject and determining concentration levels of one or more biomarker metabolites in the sample from the subject; and diagnosing the subject as having depression or an increased risk of depression when the concentration levels of the one or more biomarker metabolites in the sample from the subject is different from (greater or less) than the concentration levels the one or more biomarker metabolites in a control sample. administering an effective amount sufficient to attenuate, reduce, or eliminate the symptoms of depression of one or more first depression treatments over a period of time; obtaining an additional sample from the subject and reevaluating the concentration level of the one or more biomarker metabolites in comparison to the subject's initial concentration levels of the one or more biomarker metabolites and the control concentration levels of the one or more biomarker metabolites; evaluating the efficacy of the depression treatment; and continuing the one or more first depression treatments, administering an additional depression treatment, or administering one or more second depression treatments (switching the treatment regimen).

In one aspect, the biomarker metabolite comprises one or more of: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine; Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline; Glycerophospholipids (i.e., PC aa): PC aa C24:0; PC aa C26:0; PC aa C28:1; PC aa C30:0; PC as C32:0; PC as C32:1; PC aa C32:3; PC aa C34:1; PC aa C34:2; PC aa C34:3; PC aa C34:4; PC as C36:0; PC as C36:1; PC as C36:2; PC as C36:3; PC as C36:4; PC as C36:5; PC as C36:6; PC as C38:0; PC as C38:3; PC aa C38:4; PC aa C38:5; PC aa C38:6; PC as C40:1; PC as C40:2; PC aa C40:3; PC as C40:4; PC aa C40:5; PC as C40:6; PC as C42:0; PC aa C42:1; PC as C42:2; PC aa C42:4; PC aa C42:5; or PC aa C42:6; Glycerophospholipid ethers (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6; Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1; short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA)); 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; or combinations thereof.

In another aspect, the sample from the subject is one or more of whole blood, serum, plasma, urine, saliva, or other body fluids. In another aspect, the control sample is from an untreated subject or a subject or population of subjects not experiencing depression or not at risk for depression.

In another aspect, the biomarker metabolite concentration level increases compared to the initial sample or control. In another aspect, the biomarker metabolite concentration level decreases compared to the initial sample or control. In another aspect, two or more biomarker metabolite concentration levels covary and increase or covary and decrease (positive correlation) compared to the initial sample or control. In another aspect, two or more biomarker metabolite concentration levels vary dissimilarly (negative correlation) the initial sample or control.

In another aspect, the efficacy of the depression treatment is evaluated using the Hamilton Depression Rating Scale (HRSD17), the Quick Inventory of Depressive Symptomatology (QIDS), subscales thereof, or specific questions thereof. In another aspect, the period of time is at least: 4-weeks, 8-weeks, 12-weeks, 16-weeks, 32-weeks, 64-weeks, or greater than 64-weeks. In another aspect, the depression is Major Depression Disorder (MDD), core depression (CD+), anxious depression (ANX+), neurovegetative symptoms of melancholia (NVSM+), or subclinical characteristics associated with depression.

In another aspect, the first depression treatments comprise one or more of antidepressants, cognitive behavior therapy, exercise, dietary supplements, prebiotics, probiotics, dietary changes, or an elimination diet. In another aspect, the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram.

Another embodiment described herein is a method for screening a subject for depression or an increased risk of depression, the method comprising: obtaining a sample from the subject and determining concentration levels of one or more biomarker metabolites in the sample from the subject; and diagnosing the subject as having depression or an increased risk of depression when the concentration levels of the one or more biomarker metabolites in the sample from the subject is different from (greater or less) than the concentration levels the one or more biomarker metabolites in a control sample.

In one aspect, the method further comprises: initially treating the subject for depression by administering an effective amount sufficient to attenuate, reduce, or eliminate the symptoms of depression of one or more of antidepressants, cognitive behavior therapy, exercise, dietary supplements, prebiotics, probiotics, dietary changes, or an elimination diet for a period of time.

In another aspect, the method further comprises: obtaining an additional sample from the subject and reevaluating the concentration level of the one or more biomarker metabolites in comparison to the subject's initial concentration levels of the one or more biomarker metabolites and the control concentration levels of the one or more biomarker metabolites; evaluating the efficacy of the initial depression treatment; and continuing the one or more initial depression treatments, administering an additional depression treatment, or administering one or more second depression treatments (switching the treatment regimen).

In another aspect, the biomarker metabolite comprises one or more of: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine; Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline; Glycerophospholipids (i.e., PC aa): PC aa C24:0; PC aa C26:0; PC aa C28:1; PC as C30:0; PC as C32:0; PC as C32:1; PC as C32:3; PC as C34:1; PC aa C34:2; PC as C34:3; PC aa C34:4; PC as C36:0; PC as C36:1; PC as C36:2; PC as C36:3; PC as C36:4; PC as C36:5; PC as C36:6; PC as C38:0; PC as C38:3; PC as C38:4; PC as C38:5; PC as C38:6; PC as C40:1; PC as C40:2; PC as C40:3; PC as C40:4; PC as C40:5; PC as C40:6; PC aa C42:0; PC as C42:1; PC as C42:2; PC as C42:4; PC as C42:5; or PC as C42:6; Glycerophospholipid ethers (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6; Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1; short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA)); 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; or combinations thereof.

In another aspect, the sample from the subject is one or more of whole blood, serum, plasma, urine, saliva, or other body fluids. In another aspect, the control sample is from an untreated subject or a subject or population of subjects not experiencing depression or not at risk for depression.

In another aspect, the biomarker metabolite concentration level increases compared to the initial sample or control. In another aspect, the biomarker metabolite concentration level decreases compared to the initial sample or control. In another aspect, two or more biomarker metabolite concentration levels covary and increase or covary and decrease (positive correlation) compared to the initial sample or control. In another aspect, two or more biomarker metabolite concentration levels vary dissimilarly (negative correlation) the initial sample or control.

In another aspect, the depression is Major Depression Disorder (MDD), core depression (CD+), anxious depression (ANX+), neurovegetative symptoms of melancholia (NVSM+), or subclinical characteristics associated with depression. In another aspect, the period of time is at least: 4-weeks, 8-weeks, 12-weeks, 16-weeks, 32-weeks, 64-weeks, or greater than 64-weeks.

In another aspect, the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram.

In another aspect, the depression and efficacy of treatment is evaluated using the Hamilton Depression Rating Scale (HRSD17), the Quick Inventory of Depressive Symptomatology (QIDS), subscales thereof, or specific questions thereof. In another aspect, if the subject's depression score statistically significantly decreases from the initial score, the treatment is efficacious.

In another aspect, the method further comprises detecting at least one positively correlated biomarker metabolite, wherein detecting the at least one positively correlated biomarker metabolite is associated with a presence of at least one independent indicator of depression.

In another aspect, the positively correlated biomarker metabolite is one or more of acylcarnitine C3, acylcarnitine C5, α-aminoadipic acid, sarcosine, serotonin (5HT), or 3-methoxy-4-hydroxyphenylglycol (MHPG), or combinations thereof. In another aspect, at least one independent indicator of depression comprises a HRSD₁₇ score of >18.

In another aspect the method further comprises detecting at least one negatively correlated biomarker metabolite, wherein detecting the at least one negatively correlated biomarker metabolite is associated with an absence of at least one independent indicator of depression.

In another aspect, the baseline detection of an increased level of at least one biomarker metabolite compared to a control comprising one or more of acylcarnitine C3, acylcarnitine C5, α-aminoadipic acid, sarcosine, serotonin or 3-methoxy-4-hydroxyphenylglycol (MHPG), or combinations thereof indicates the subject has at least one independent indicator of depression.

Another embodiment described herein is a method for predicting a depression subject's propensity of treatment success (depression remission) in response to an antidepressant, the method comprising: obtaining a sample from the subject and evaluating the concentration level of the one or more biomarker metabolites in comparison to the control concentration levels of the one or more biomarker metabolites; administering an effective amount sufficient to attenuate, reduce, or eliminate the symptoms of depression of one or more antidepressants, cognitive behavioral therapy, or a combination thereof for a period of time; obtaining an additional sample from the subject and evaluating the concentration level of the one or more biomarker metabolites in comparison to the subject's initial concentration levels of the one or more biomarker metabolites and the control concentration levels of the one or more biomarker metabolites; continuing the one or more initial depression treatments, administering an additional depression treatment, or administering one or more second depression treatments (switching the treatment regimen).

In one aspect, the biomarker metabolite comprises one or more of: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine; Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline; Glycerophospholipids (i.e., PC aa): PC aa C24:0; PC aa C26:0; PC aa C28:1; PC aa C30:0; PC as C32:0; PC aa C32:1; PC aa C32:3; PC aa C34:1; PC aa C34:2; PC aa C34:3; PC aa C34:4; PC as C36:0; PC aa C36:1; PC aa C36:2; PC aa C36:3; PC aa C36:4; PC as C36:5; PC aa C36:6; PC as C38:0; PC as C38:3; PC aa C38:4; PC aa C38:5; PC aa C38:6; PC as C40:1; PC as C40:2; PC aa C40:3; PC as C40:4; PC aa C40:5; PC aa C40:6; PC as C42:0; PC aa C42:1; PC aa C42:2; PC aa C42:4; PC aa C42:5; or PC aa C42:6; Glycerophospholipid ethers (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6; Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1; short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA)); 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; or combinations thereof.

In another aspect, the sample from the subject is one or more of whole blood, serum, plasma, urine, saliva, or other body fluids. In another aspect, the control sample is from an untreated subject or a subject or population of subjects not experiencing depression or not at risk for depression.

In another aspect, the depression is Major Depression Disorder (MDD), core depression (CD+), anxious depression (ANX+), neurovegetative symptoms of melancholia (NVSM+), or subclinical characteristics associated with depression. In another aspect, the period of time is at least: 4-weeks, 8-weeks, 12-weeks, 16-weeks, 32-weeks, 64-weeks, or greater than 64-weeks. In another aspect, the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, wherein the antidepressant comprises escitalopram or citalopram. In another aspect, the depression and efficacy of treatment is evaluated using the Hamilton Depression Rating Scale (HRSD17), the Quick Inventory of Depressive Symptomatology (QIDS), subscales thereof, or specific questions thereof.

In another aspect, if the subject has greater baseline concentration levels of acylcarnitines C3, C5, α-aminoadipic acid, sarcosine, or serotonin in comparison to control concentration levels, and after at least 8-weeks of treatment these biomarker metabolites had concentrations greater than control levels, the subject has a propensity of antidepressant treatment success.

In another aspect, if the subject has greater baseline concentration levels of acylcarnitines C3, C5, α-aminoadipic acid, sarcosine, or serotonin in comparison to control concentration levels, and the concentration levels of these biomarker metabolites remains greater than baseline after 8 weeks of antidepressant treatment, the subject has a propensity of antidepressant treatment success.

In another aspect, if the subject has greater baseline concentration levels of one or more of 3-methoxy-4-hydroxyphenylglycol (MHPG) and serotonin (5HT) compared to control concentration levels and after at least 8-weeks of antidepressant treatment, the levels of MHPG and 5HT decreased compared to the baseline concentration levels, the subject has a propensity of antidepressant treatment success.

In another aspect, if the subject has greater concentrations levels of one or more of PC aa C34:1, PC aa C34:2, PC aa C36:2 and PC aa C36:4 compared to baseline concentration levels after at least 8-weeks of antidepressant treatment, the subject has a propensity of antidepressant treatment success.

In another aspect, if the subject has greater concentration levels after at least 8-weeks of antidepressant treatment of one or more of acylcarnitine (C5-M-DC), histidine, proline, kynurenine and trans-4-hydroxyproline, PC aa C34:1, PC as C34:2, PC aa C36:1, PC aa C36:2, PC aa C36:3, PC aa C36:4, PC aa C40:1, PC ae C34:2, PC ae C34:3, PC ae C36:1, PC ae C36:2, PC ae C36:3, PC ae C38:2, or PC ae C38:3, and the concentration levels of one or more of these biomarker metabolites are inversely associated with changes in the subject's depression score, the subject has a propensity of antidepressant treatment success.

Another embodiment described herein is a method for evaluating a depression subject's response to antidepressant treatment, cognitive behavioral therapy or a combination thereof, the method comprising: obtaining a sample from the subject and evaluating the concentration level of the one or more biomarker metabolites in comparison to the control concentration levels of the one or more biomarker metabolites; administering an effective amount sufficient to attenuate, reduce, or eliminate the symptoms of depression of one or more antidepressants, cognitive behavioral therapy, or a combination thereof for a period of time; obtaining an additional sample from the subject and evaluating the concentration level of the one or more biomarker metabolites in comparison to the subject's initial concentration levels of the one or more biomarker metabolites and the control concentration levels of the one or more biomarker metabolites; evaluating the efficacy of the initial depression treatment; and continuing the one or more initial depression treatments, administering an additional depression treatment, or administering one or more second depression treatments (switching the treatment regimen).

In one aspect, the biomarker metabolite comprises one or more of: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine; Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline; Glycerophospholipids (i.e., PC aa): PC aa C24:0; PC aa C26:0; PC aa C28:1; PC aa C30:0; PC as C32:0; PC aa C32:1; PC as C32:3; PC aa C34:1; PC aa C34:2; PC aa C34:3; PC aa C34:4; PC aa C36:0; PC aa C36:1; PC as C36:2; PC aa C36:3; PC aa C36:4; PC aa C36:5; PC aa C36:6; PC as C38:0; PC as C38:3; PC aa C38:4; PC as C38:5; PC aa C38:6; PC as C40:1; PC as C40:2; PC as C40:3; PC as C40:4; PC aa C40:5; PC aa C40:6; PC as C42:0; PC aa C42:1; PC as C42:2; PC aa C42:4; PC as C42:5; or PC aa C42:6; Glycerophospholipid ethers (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6; Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1; short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (ds-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA)); 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; or combinations thereof.

In another aspect, the sample from the subject is one or more of whole blood, serum, plasma, urine, saliva, or other body fluids. In another aspect, the control sample is from an untreated subject or a subject or population of subjects not experiencing depression or not at risk for depression.

In another aspect, the depression is Major Depression Disorder (MDD), core depression (CD+), anxious depression (ANX+), neurovegetative symptoms of melancholia (NVSM+), or subclinical characteristics associated with depression. In another aspect, the period of time is at least: 4-weeks, 8-weeks, 12-weeks, 16-weeks, 32-weeks, 64-weeks, or greater than 64-weeks. In another aspect, the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram. In another aspect, the efficacy of the depression treatment is evaluated using the Hamilton Depression Rating Scale (HRSD₁₇), the Quick Inventory of Depressive Symptomatology (QIDS), subscales thereof, or specific questions thereof.

In another aspect, after at least 8-weeks of antidepressant treatment, the subject has: greater concentration levels compared to baseline concentration levels of acylcarnitine C3, C4 and C5; arginine, proline, methionine sulfoxide; phosphatidylcholines (PC as C36:1, C30:0, C42:2); phosphatidylcholine ethers (PC ae C34:3, C38:2, C36:3); and sphingomyelin SM C24:0; and decreased concentration levels compared to baseline of acylcarnitines C8, C10, C12, C14:2, C16, C16:1, C18, C18:1, and C18:2; serotonin and sarcosine.

In another aspect, after at least 8-weeks of antidepressant treatment, the subject has: greater concentration levels compared to baseline levels of acylcarnitine indoles in TRP metabolism; 5-Hydroxytryptophan (THTP), 5-hydroxyindoleacetic acid (5HIAA), indole-3-acetic acid (I3AA); homogentisic acid (HGA), vanillylmandelic acid, 4-hydroxyphenylacetic acid (4HPAC), 4-hydroxybenzoic acid (4HBAC), guanine (G), paraxanthine (PXAN), xanthosine (XANTH), salicylic acid (SA), the ratio of 4-hydroxyphenylacetic acid to tyrosine, the ratio of 5-hydroxyindoleacetic acid to serotonin, the ratio of uric acid to xanthosine, and the ratio of paraxanthine to xanthosine; and decreased concentration levels of serotonin, 3-methoxy-4-hydroxyphenylethyleneglycol (MHPG), xanthosine, hypoxanthine, and the ratio of 3-methoxy-4-hydroxyphenylethyleneglycol to tyrosine.

In another aspect, after at least 8-weeks of cognitive behavioral therapy, the subject has: greater concentration levels compared to baseline levels of acylcarnitine (C0, C3, C4, and C5), α-aminoadipic acid, glutamate, and proline, isoleucine, aspartic acid, histidine, valine, tryptophan, tyrosine, phenylalanine, methionine, methionine-sulfoxide, and sarcosine; acylcarnitines (C10:2, C161-OH, C16-OH, C3:1, C3-OH, C4:1, C5:1, C5:1-DC, C5-OH (C3-DC-M), C6:1, C9); Phosphatidylcholines: (PCaa C24:0, C28:1, C30:0, C32:0, C32:1, C32:3, C34:1, C34:2, C34:3, C34:4, C36:1, C36:2, C36:3, C36:4, C36:5, C36:6, C38:0, C38:3, C38:4, C38:5, C38:6, C40:2, C40:4, C40:5, C40:6, C42:0, C42:1, C42:2, C42:4, C42:5, C42:6); (PC ae C30:2, C32:1, C32:2, C34:0, C34:1, C34:2, C34:3, C36:0, C36:1, C36:2, C36:3, C36:4, C36:5, C38:0, C38:2, C38:3, C38:4, C38:5, C38:6, C40:1, C40:2, C40:3, C40:4, C40:5, C40:6, C42:1, C42:2, C42:3, C42:4, C42:5, C44:3, C44:4, C44:5, C44:6); (LysoPC a C24:0, C26:0, C26:1, C28:0, C28:1); Sphingomyelins: SM (OH) C14:1, C22:1, C24:1, SM C16:0, C16:1, C18:0, C24:0, C24:1, C26:0, C26:1; and decreases in the subject's depression score.

Another embodiment described herein is a method for differentiating a depression subject's depression phenotype, the method comprising: identifying a subject experiencing depression or at risk of depression; obtaining a sample from the subject and evaluating the concentration level of the one or more biomarker metabolites in comparision to the control concentration levels of the one or more biomarker metabolites; administering an effective amount sufficient to attenuate, reduce, or eliminate the symptoms of depression of one or more antidepressants, cognitive behavioral therapy, or a combination thereof for a period of time; obtaining an additional sample from the subject and evaluating the concentration level of the one or more biomarker metabolites in comparison to the subject's initial concentration levels of the one or more biomarker metabolites and the control concentration levels of the one or more biomarker metabolites; evaluating the phenotype and efficacy of the initial depression treatment; and continuing the one or more initial depression treatments, administering an additional depression treatment, or administering one or more second depression treatments (switching the treatment regimen).

In one aspect, the biomarker metabolite comprises one or more of: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine; Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline; Glycerophospholipids (i.e., PC aa): PC aa C24:0; PC aa C26:0; PC as C28:1; PC aa C30:0; PC aa C32:0; PC aa C32:1; PC aa C32:3; PC aa C34:1; PC aa C34:2; PC aa C34:3; PC aa C34:4; PC aa C36:0; PC aa C36:1; PC aa C36:2; PC aa C36:3; PC aa C36:4; PC aa C36:5; PC aa C36:6; PC aa C38:0; PC aa C38:3; PC aa C38:4; PC aa C38:5; PC aa C38:6; PC aa C40:1; PC aa C40:2; PC as C40:3; PC aa C40:4; PC aa C40:5; PC aa C40:6; PC aa C42:0; PC aa C42:1; PC aa C42:2; PC aa C42:4; PC aa C42:5; or PC aa C42:6; Glycerophospholipid ethers (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6; Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1; short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cas-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA)); 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; or combinations thereof.

In another aspect, the sample from the subject is one or more of whole blood, serum, plasma, urine, saliva, or other body fluids. In another aspect, the control sample is from an untreated subject or a subject or population of subjects not experiencing depression or not at risk for depression.

In another aspect, the depression is Major Depression Disorder (MDD), core depression (CD+), anxious depression (ANX+), neurovegetative symptoms of melancholia (NVSM+), or subclinical characteristics associated with depression. In another aspect, the period of time is at least: 4-weeks, 8-weeks, 12-weeks, 16-weeks, 32-weeks, 64-weeks, or greater than 64-weeks. In another aspect, the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram. In another aspect, the type of depression (phenotype) and efficacy of the depression treatment is evaluated using the Hamilton Depression Rating Scale (HRSD₁₇), the Quick Inventory of Depressive Symptomatology (QIDS), subscales thereof, or specific questions thereof.

In another aspect, the baseline detection of an decreased concentration level of at least one biomarker metabolite compared to a control comprising one or more of acylcarnitines C0, C3, C18m, C5:1, C5-DC/C6-OH, C16-OH, or combinations thereof indicates the subject has at least one independent indicator of core depression (CD+).

In another aspect, the baseline detection of an decreased concentration level of at least one biomarker metabolite compared to a control comprising one or more of acylcarnitines C5-DC/C6-OH and C16-OH, or combinations thereof and higher concentration levels of acylcarnitine C10 compared to a control, indicates the subject has at least one independent indicator of neurovegetative symptoms of melancholia (NVSM+).

In another aspect, after at least 8-weeks of antidepressant treatment, subjects having core depression (CD+), had greater concentration levels of acylcarnitines C5-DC/C6-OH, C5-M-DC, C6, C14:1, C16, C16-OH, C16:1, and C18:1-OH compared with concentration levels of subjects having neurovegetative symptoms of melancholia (NVSM+).

In another aspect, after at least 8-weeks of antidepressant treatment, subjects having core depression (CD+), had lower concentration levels of acylcarnitines C2, C3 and C6 compared with anxious depression (ANX+) subjects.

In another aspect, after at least 8-weeks of antidepressant treatment, subjects having neurovegetative symptoms of melancholia (NVSM+), had lower concentration levels of acylcarnitines C0, C2, C3, C5-DC/C6-OH, C5-M-DC, C6, C10, C14:1, C16, C16-OH, C16:1 and C18:1-OH compared with anxious depression (ANX+) subjects.

In another aspect, after at least 8-weeks of antidepressant treatment, subjects having core depression (CD+), had increased concentration levels of acylcarnitines C0, C3, C4, C5-M-DC and C5:1 compared with baseline concentration levels of these biomarker metabolites.

In another aspect, after at least 8-weeks of antidepressant treatment, subjects having core depression (CD+) or anxious depression (ANX+), had increased concentration levels of acylcarnitines C0, C3, and C4 compared with baseline concentration levels of these biomarker metabolites.

In another aspect, after at least 8-weeks of antidepressant treatment, subjects having core depression (CD+) or neurovegetative symptoms of melancholia (NVSM+), had decreased concentration levels of acylcarnitines C8, C10, and C12 compared with baseline concentration levels of these biomarker metabolites.

In another aspect, after at least 8-weeks of antidepressant treatment, subjects having core depression (CD+) or neurovegetative symptoms of melancholia (NVSM+), had decreased concentration levels of acylcarnitines C14:1, C14:2, C16, C16-OH, C16:1, C18, C18:1, C18:1-OH, C18:2 compared with baseline concentration levels of these biomarker metabolites.

In another aspect, after at least 8-weeks of antidepressant treatment, subjects having core depression (CD+), neurovegetative symptoms of melancholia (NVSM+) or anxious depression (ANX+), had decreased concentration levels of acylcarnitines, C5-OH, C16:1, C18:1, and C18:2, compared with baseline concentration levels of these biomarker metabolites.

### DESCRIPTION OF THE DRAWINGS

FIG. 1: Role of acylcarnitines in β-oxidation of fatty acids. FIG. 1A. Mitochondrial energy metabolism pathways involve: branched-chain amino acids (BCAAs), acyl carnitines, fatty acid beta-oxidation and tricarboxylic acid cycle (TCA cycle). A delicate balance between two of the main cellular energy resources, glucose utilization and fatty acid utilization, is essential to maintain physiological homeostasis, the perturbation of which may lead to pathologic processes. The grey font metabolites have not been measured in p180. Abbreviations: Leu: Leucine, Val: Valine, lie: Isoleucine, CoA: Coenzyme A, CPT1: carnitine-palmitoyl-transferase 1, CPT2: carnitine-palmitoyl-transferase 2, CACT: carnitine acylcarnitine-translocase, CrAT: carnitine O-acetyltransferase. Metabolite abbreviations are spelled out in Table X. FIG. 1B: Effect of SSRI on interconnected pathways, including polyamine, sarcosine, urea cycle and 1-carbon metabolism. Orange boxes indicate an increase in metabolite levels, while blue boxes represent a decrease in metabolite levels. White boxes represent no change in in metabolite levels. Metabolites not measured in this assay are in grey. Abbreviations: NH3: Ammonia, C02: Carbon Dioxide, SAH: S-adenosyl-I-homocysteine, SAM: S-adenosyl methionine, Cit: Citrulline, Om: Omithine, Arg: Arginine, ADMA: Asymmetric dimethylarginine, CoA: Coenzyme A.

FIG. 2. Heat map depicting log 2-fold changes in metabolite levels P-values were obtained using linear mixed effect models controlling for age, sex and baseline HRSD₁₇ were corrected for multiple comparisons. Red indicates an increase and blue indicates a decrease in metabolite levels over 8 weeks of treatment; *: q-value<0.05, **: q-value <0.01 and **: q-value <0.001. Abbreviations: PC: Phosphatidylcholine, SM: Sphingomyelin, LysoPC: Lyso-Phosphatidylcholines, SSRI: Selective Serotonin Reuptake Inhibitor, HRSD₁₇: 17-item Hamilton Rating Scale for Depression.

FIG. 3. Hierarchical clustering of Spearman's rank correlation of change in metabolite levels Red represents positive correlations and blue represents negative correlations. Cluster #1: enriched for medium and long-chain acylcarnitines (e.g., C8, C9, C10, C12, C16), Cluster #2: biogenic amines including serotonin, spermine, spermidine, taurine, putrescine, glutamate, omithine and sarcosine. Cluster #3: short chain-acylcarnitines (C3, C4, C5), branched-chain amino acids (isoleucine and valine), kynurenine, arginine, glycine, tryptophan and glutamine. Cluster #4: long-chain phosphatidylcholine of PC ae class and Cluster #5: a large number of phospholipids and lyso-phospholipids. Abbreviations: PC: Phosphatidylcholine, LysoPC: Lyso-Phosphatidylcholines, SM: Sphingomyelin.

FIG. 4. Metabolite Concentrations Across Visits stratified by Response Group. Levels (log 2 transformed) of metabolites at pre-treatment and 8 weeks post-treatment. Green lines indicate participants who remitted and red lines indicate participants who failed to respond to the treatment at the end of therapy. Asterisks indicate statistical significance of mean differences between the two groups (unadjusted p<0.05). Error bars represent standard error of the means. Black stars represent statistical significance at visit. P-values were obtained from linear mixed effect models corrected for age, sex, antidepressant and 17-item Hamilton Rating Scale for Depression scores.

FIG. 5. Serotonin Neighbors in Partial Correlation Networks Computed from Baseline and Week 8 Metabolic Profiles. Vertices represent metabolites. Edges represent partial correlations that surpassed Bonferroni significance level. Solid lines represent positive partial correlation and dashed lines represent negative partial correlation.

FIG. 6. Venn diagram of the Overlap between (Mayo data and Emory data) positive phenotype [Core Depression (CD), Neurovegetative Symptoms of Melancholia (NVSM), and anxiety (ANX)]. Circles represent each group of the phenotype (CD, NVSM and ANX). The numbers inside overlapping circles represent patients who achieved the positive phenotype and the overlap between them.

FIG. 7. Eight Weeks Signature of Exposure to Escitalopram/Citalopram Stratified by Phenotype. Heatmap depicts log 2 fold change of metabolite levels from baseline to 8 weeks of SSRI treatment. P-values were obtained using linear mixed effect models controlling for age, sex, treatment and HRSD₁₇. Red indicate an increase and blue indicate a decrease in metabolite levels over 8 weeks of treatment; *: p-value <0.05, *: p-value <0.01, and **: p-value <0.001.

FIGS. 8A-C. Acylcarnitine concentrations. A. Short-chain Acylcarnitine concentrations. Baseline, 4 weeks and 8 weeks stratified by Phenotype (+) and (−). B. Medium-chain Acylcarnitine concentrations. Baseline, 4 weeks and 8 weeks stratified by Phenotype (+) and (−). C. Long-chain Acylcarnitine concentrations. Baseline, 4 weeks and 8 weeks stratified by Phenotype (+) and (−). Error bars represent standard error of the means in all panels.

FIG. 9. Metabolic Signature of Eight Weeks of Exposure to SSRI. The heatmap depicts the log 2 fold change of metabolite levels from baseline to week eight of SSRI treatment. CD+: Core Depression, ANX+: Anxiety, NVSM+: Neurovegetative Symptom of Melancholia. P-values were obtained using linear mixed effect models controlling for age, sex and baseline 17-item Hamilton Rating Scale for Depression scores. Red indicates an increase and blue indicates a decrease in metabolite levels over eight weeks of treatment; *: p-value <0.05, *: p-value <0.01 and **: p-value<0.001.

FIG. 10. Workflow for data analysis.

FIG. 11. A heatmap showing correlations between the metabolite modules and the changes in symptom severity (HRSD₁₇) scores. Each module represents a number of metabolites that showed strongly correlated perturbation patterns, from baseline to week 12, in response to CBT. The metabolite members of the three modules, purple, yellow and green-yellow, that showed significant association (R2>0.3, at p<0.1) with changes in HRSD₁₇ scores are presented in adjacent boxes.

FIGS. 12A-11B. Purple module characteristics. 12A. The pairwise correlations between the changes in the metabolite members to each other and also to HRSD₁₇ changes are shown in a composite plot. 12B. The trajectories of each member metabolite with its mean (±SEM) at baseline and week 12 are presented for remitters and treatment failures.

FIG. 13A-13B. Yellow module characteristics. 13A. The pairwise correlations between the changes in the metabolite members to each other and also to HRSD₁₇ changes are shown in a composite plot. 13B. The trajectories of each member metabolite with its mean (t SEM) at baseline and week 12 are presented for remitters and treatment-failures.

FIG. 14A-14B. 14A. A heatmap showing correlations between the metabolite modules and the changes in symptom severity (HRSD₁₇) scores, highlighting the green-yellow module. The member metabolites of this large module are presented in the adjacent box. 14B. A heatmap showing the pairwise Pearson correlations between the member metabolites of the green-yellow module. The empty cells indicate that the p-values of the correlations for that metabolite-pair was not statistically significant (p>0.1). The trajectories of each member metabolite of the green-yellow module, consisting of mostly lipids, or lipid-elated metabolites, with its mean (+/−SEM) at baseline and week 12 are presented for remitters and treatment-failures.

FIGS. 15A-15B. Metabolic signature of drug exposure. 15A. Shows the heatmap of metabolite changes at baseline, week 4, and week 8, normalized to baseline levels. 15B. Shows changes within the purine, tryptophan, and tyrosine pathways.

FIGS. 16A-16F. Metabolite changes associated with HRSD₁₇ scores. 16A. 5HT (Serotonin). 16B. 5 HIAA/5HT ratio. 16C. 5HIAA (5-hydroxyindoleacetic acid). Temporal changes in 16D. HRSD₁₇ scores. 16E. 5HT. 16F. MHPG differed significantly between responders and nonresponders. The error bars represent standard error of the mean.

FIGS. 17A-17F. Partial correlation networks (PCN). 17A. PCN at baseline. 17B. PCN at baseline after 1000 bootstrap estimations. 17C. PCN at week 8. 17D. PCN at week 8 after 1000 bootstrap estimations. The different clusters representing communities of closely associated metabolites are shown in different colors. Differential PCN as a function of high versus low HRSD₁₇ week 8 scores at baseline (17E) and week 8 (17F). The edges between metabolites most impacted by higher HRSD₁₇ week 8 scores are bolded in green, while those by lower HRSD₁₇ week 8 scores are bolded in red.

FIGS. 18A-18B. Plots showing regional association plots generated with SNiPA. 18A. The HGA/GR ratio at baseline. 18B. The MET/TYR ratio at week 8.

FIG. 19. Baseline profile difference in Body Mass Index.

FIG. 20. Baseline profile differences in depression severity: HRSD₁₇<20 vs ≥20.

FIGS. 21A-21D. Insomnia and Hypersomnia: QIDS-C items 1-3 vs. QIDS-C item 4. 21A. Falling asleep. 21B. Sleep during the Night. 21C. Waking up too early. 21D. Sleeping too much.

FIGS. 22A-22B. Baseline profile differences in insomnia and hypersomnia. Models were corrected for age, sex and BMI. A. Model 1: Scores (1, 2, 3) vs 0. B. Model 2: Score (2 and 3) vs. (0 and 1).

FIG. 23. Baseline Profile Differences: Anxious vs Non-Anxious comorbid disorder and Anxiety—High Anxious vs Low Anxious (HRSA Total-cut point 15).

FIGS. 24A-24B. Assessment of Decreased appetite vs. increased appetite. 24A. Decreased appetite. 24B. Increased appetite.

FIGS. 25A-25B. Baseline profile difference, decreased appetite and increases appetite.

FIG. 26. Baseline profile differences low energy vs. no-change.

FIGS. 27A-27B. Assessment of slowed down vs. no-change. 27A. Feeling slowed down. 27B. Feeling restless.

FIG. 28. Baseline profile differences for psychomotor changes: slowed down and restless vs. no-change.

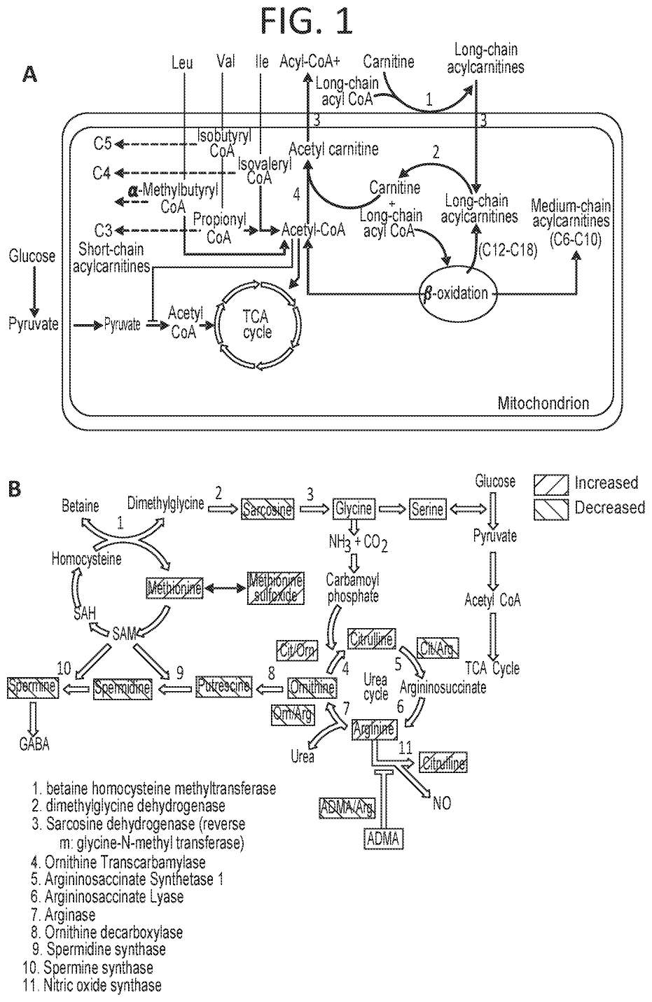

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Further, unless otherwise required by context, singular terms shall include pluralities and plural terms shall include the singular.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Subject” and “patient” as used herein interchangeably refers to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

As used herein, “depression” refers to a mood disorder that causes a persistent feeling of sadness and loss of interest. As used herein “depression” includes subclinical characteristics associated with depression such as sadness, loss of interest in activities, loss of appetite, anhedonia, insomnia, changes in sleep, difficulty falling asleep, waking during the night, restless sleep, waking too early, sleeping too much, low energy level, lack of concentration, diminished or altered daily behavior, low self-esteem, suicidal thoughts, or anxiety coupled with depression.

As used herein, “sample,” “test sample,” and “biological sample” refer to fluid sample containing or suspected of containing a biomarker metabolite. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing a biomarker metabolite may be assayed directly. In one embodiment, the source containing a biomarker metabolite is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis.

As used herein a biomarker metabolite comprises one or more metabolites that is a measurable indicator of the severity or presence of a disease state, disorder, or physiological or metal state of a subject. In one aspect as used herein, biomarker metabolites are acylcarnitines (short-, medium-, and long-chain); amino acids; biogenic amines, glycerophospholipids, sphingolipids, tryptophan metabolites, phenylalanine metabolites, tyrosine metabolites, sulfur-amino acid metabolites, tocopherol metabolites, purine metabolites, or other metabolites.

As used herein, the terms “treat”, “treating,” or “treatment” of any disease or disorder refer In an embodiment, to ameliorating the disease or disorder (i.e., slowing or arresting or reducing the development of the disease or at least one of the clinical symptoms thereof). In an embodiment, “treat,” “treating,” or “treatment” refers to alleviating or ameliorating at least one physical or mental parameter including those which may not be discernible by the patient.

As used herein, the term “effective amount” refers to the amount of a treatment sufficient to cure, ameliorate the disease or disorder (i.e., slowing or arresting or reducing the development of the disease or at least one of the clinical symptoms thereof), or cause remission of the symptoms.

As used herein, the term “preventing” refers to a reduction in the frequency of, or delay in the onset of, symptoms of the condition or disease.

As used herein, a subject is “in need of” a treatment if such subject would benefit biologically, medically, or in quality of life from such treatment.

The term “prophylaxis” refers to preventing or reducing the progression of a disorder, either to a statistically significant degree or to a degree detectable to one skilled in the art.

As used herein, the terms “efficacy” or “efficacious” refer to the success of a treatment in bringing about a cure, amelioration, remission, or reduction of symptoms of a disease or disorder.

As used herein, the terms “depression instrument scores” or “depression scores” refer to the “Hamilton Depression Rating Scale” or “HRSD₁₇”, the “Quick Inventory of Depressive Symptomatology” or “QIDS” (e.g., QIDS SR-16), subscales thereof, or specific questions thereof. These instruments are useful for evaluating the type (phenotype) and severity of depression. The HRSD₁₇ method was described by Hamilton, Br. J. Soc. Clin. Psychol. 6(4): 278-96 (1967), which is incorporated by reference herein for such teachings. The QIDS SR inventory was described by Rush et al., Biol. Psychiatry 54(5):573-583 (2003), which is incorporated by reference herein for such teachings. The “depression instrument scores” or “depression scores” as used herein, can be used to diagnose depression, evaluate the severity of depression, determine the phenotype of depression, and evaluate the efficacy of depression treatments.

As used herein the phrase “propensity of success” refers a subject's ability to respond to a treatment and have efficacious results including remission, cure, or a reduction of symptoms.

The term “substantially” as used herein means to a great or significant extent, but not completely.

As used herein, all percentages (%) refer to mass (or weight, w/w) percent unless noted otherwise.

The term “about” as used herein refers to any values, including both integers and fractional components that are within a variation of up to *10% of the value modified by the term “about.”

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

Terms such as “include,” “including,” “contain,” “containing,” “having,” and the like mean “comprising.”

The term “or” can be conjunctive or disjunctive.

Described herein is a targeted metabolomics approach to derive insights into mechanisms of action of antidepressants, such as escitalopram and citalopram, and the sources of variation in response to antidepressant (e.g., SSRI) treatment. The study measured 180 metabolites within three major components of the metabolome that previous work has implicated in either the pathophysiology of MDD or its response to antidepressant treatments: (1) acylcarnitines (ACs), which are involved in mitochondrial function and beta oxidation, major source for energy production; (2) amino acids and biogenic amines, which are crucial for neurotransmission; and (3) lipids involved in in membrane structure, function, and signaling, including the sphingomyelins (SM), lysophosphatidylcholines (lysoPC), and glycerophospholipids. The result described herein show how biomarker metabolites can be used to screen subjects for the likelihood of developing depression, the diagnosis thereof, monitoring the efficacy of treatment, and evaluating a subject's propensity for responding to treatment.

Plasma samples were obtained for metabolomic analyses from outpatients with non-psychotic MDD in the Mayo Pharmacogenomic Research Network Antidepressant Medication Pharmacogenomic Study (PGRN-AMPS) (Mrazek et al., J. Clin. Psychopharmacol. 34, 313-317 (2014)). Samples were collected at baseline and at 4 and 8 weeks during acute phase treatment with either escitalopram (n=161) or citalopram (n=108).

The study compared and contrasted these three phenotypes to ascertain whether they are biochemically distinct either at baseline in the symptomatic state or in-terms of biochemical differences following 8 weeks of acute treatment with an SSRI.

Acylcarnitines (AC) were analyzed because of their central role in the CNS and their relevance to depression. In animal studies, an incomplete β-oxidation of fatty acids shows elevated medium- and long-chain acylcarnitines in the depressed rats. AC is involved in mitochondrial function and energy, anti-oxidation and membrane stability, gene expression and neurotransmission, thus it widely considered as neuroprotective. A genetic defect in mitochondrial β-oxidation is used to be diagnosed by alterations in acylcarnitine levels. Studies has shown an altered mitochondrial function or reduced ATP production in patients with lifetime diagnosis of MDD, or patients with known mitochondrial disorders frequently have depressive symptoms, or lifetime diagnosis of MDD.

Acetyl-L-carnitine levels appear to be lower in patients with MDD (n=71) as compared to healthy controls (n=45). Subjects with MDD and HIV-positive or negative have decreased level acylcarnitines (propionylcarnitine, isobutyrylcarnitine, isovalerylcarnitine, and 2-methylbutyrylcarnitine) and were correlated with depressive symptom severity. Moreover, patients with uremia undergoing hemodialysis had decrease in their self-rating depression scale and increased total, free, and acylcarnitine levels after L-carnitine treatment. ACs are highly expressed in the brain—especially the hypothalamus. Shug et al., Life Sci. 31(25): 2869-2874 (1982); Bresolin et al., Exp. Neurol. 78(2): 285-292 (1982). This intra-cellular compound operates as source of the acetyl groups during β-oxidation, and it assists the transfer of fatty acids from cytosol to mitochondria.

Biomarker metabolites were analyzed in human plasma samples using the Biocrates AbsoluteIDQ® p180 mass-spectrometry-based targeted metabolomics kit (Biocrates, Austria), which assays 180 metabolites including amino acids (21); biogenic amines (21); monosaccharide hexoses (1); acylcarnitines (40); glycerophospholipids (90), and sphingomyelins (15).

Other analyses (Example 3) used a targeted, liquid chromatography-electrochemical coulometric array (LCECA) metabolomics platform to assay metabolites in plasma samples according to known methods. See Matson et al., Clin. Chem. 30, 1477-1488 (1984).

One embodiment described herein is a method for diagnosing or detecting depression in a subject, the method comprising: obtaining a sample from a subject and determining concentration levels of one or more biomarker metabolites in the sample from the subject; and diagnosing the subject as having depression or an increased risk of depression when the concentration levels of the one or more biomarker metabolites in the sample from the subject is different from (greater or less) than concentration levels the one or more biomarker metabolites in a control sample.

In one aspect, the method further comprises: initially treating the subject for depression by administering an effective amount sufficient to attenuate, reduce, or eliminate the symptoms of depression of one or more of antidepressants, cognitive behavior therapy, exercise, dietary supplements, prebiotics, probiotics, dietary changes, or an elimination diet; obtaining a second sample from the subject and determining concentration levels of one or more biomarker metabolites in the sample from the subject; evaluating the concentration level of the one or more biomarker metabolites in comparison to control concentration levels of the one or more biomarker metabolites; evaluating the efficacy of the depression treatment; and continuing the one or more initial depression treatments; administering one or more additional depression treatment; or administering one or more second depression treatments (switching the treatment regimen).

In one aspect, the biomarker metabolites are acylcarnitines (short-, medium-, and long-chain); amino acids; biogenic amines, glycerophospholipids, sphingolipids, tryptophan metabolites, phenylalanine metabolites, tyrosine metabolites, sulfur-amino acid metabolites, tocopherol metabolites, purine metabolites, or other metabolites. In another aspect, the biomarker metabolites are one or more of: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine; Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline; Glycerophospholipids (i.e., PC aa): PC as C24:0; PC as C26:0; PC aa C28:1; PC as C30:0; PC as C32:0; PC as C32:1; PC as C32:3; PC as C34:1; PC as C34:2; PC as C34:3; PC as C34:4; PC as C36:0; PC as C36:1; PC as C36:2; PC as C36:3; PC as C36:4; PC as C36:5; PC as C36:6; PC as C38:0; PC as C38:3; PC as C38:4; PC as C38:5; PC as C38:6; PC as C40:1; PC as C40:2; PC as C40:3; PC as C40:4; PC as C40:5; PC as C40:6; PC as C42:0; PC as C42:1; PC as C42:2; PC as C42:4; PC as C42:5; or PC as C42:6; Glycerophospholipid ethers (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6; Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1; short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cs-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA)); 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; or combinations thereof.

In one aspect, the biomarker metabolite concentration level is less than the control concentration level. In another aspect, two or more biomarker metabolite concentration levels covary and are greater than the control or covary and are less than the control concentration levels (positive correlation). In another aspect, two or more biomarker metabolite concentration levels vary dissimilarly (negative correlation) compared to the control concentration levels.

Samples from the subject can comprise one or more of whole blood, serum, plasma, urine, saliva, or other body fluids. The control sample is from an untreated subject or a subject or population of subjects not experiencing depression or not at risk for depression. In one aspect, the depression is Major Depression Disorder (MDD), core depression (CD+), anxious depression (ANX+), neurovegetative symptoms of melancholia (NVSM+), or subclinical characteristics associated with depression.

Antidepressants for treating the subject can comprise tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In one aspect, the antidepressant is one or more selective serotonin reuptake inhibitors (SSRI). In another aspect, the antidepressant comprises an SSRI selected from escitalopram, citalopram, fluoxetine, sertraline, paroxetine, fluvoxamine, vilazodone, vortioxetine, or duloxetine. In another aspect, the antidepressant comprises an SSRI comprising escitalopram or citalopram.

Cognitive behaviorial therapy can be used to treat subjects for depression. Cognitive behavioral therapy (CBT) is a form of psychotherapy that focuses on modifying dysfunctional emotions, behaviors, and thoughts by interrogating and uprooting negative or irrational beliefs. CBT is appropriate for people of all ages, including children, adolescents, and adults. CBT can address numerous conditions, such as major depressive disorder, anxiety disorders, post-traumatic stress disorder, eating disorders, obsessive-compulsive disorders, and many others. CBT can be effective in a brief period of time, generally 5 to 20 sessions, though there is no set timeframe.

In another aspect, depression can be treated by having the subject exercise, consuming dietary supplements, prebiotics, probiotics, dietary changes, or an elimination diet that augments metabolites that have low plasma concentrations levels during periods of depression but higher levels during periods of remission or alternatively have high concentrations during depression and low concentrations during periods of remission.

In another aspect, the Hamilton Depression Rating Scale (HRSD₁₇) is used to diagnose depression, evaluate the severity of depression, determine the phenotype of depression, and evaluate the efficacy of depression treatments.

Another embodiment described herein is a method of treating depression and evaluating the treatment efficacy comprising: identifying a subject suffering from or at risk of developing depression; administering an effective amount sufficient to attenuate, reduce, or eliminate the symptoms of depression of one or more antidepressants, cognitive behavior therapy, exercise, dietary supplements, prebiotics, probiotics, or dietary changes, or a combination thereof over a period of time; obtaining a sample from the subject and evaluating concentration levels of one or more biomarker metabolites; evaluating the concentration level of the one or more biomarker metabolites in comparison to control concentration levels of the one or more biomarker metabolites; evaluating the efficacy of the depression treatment; and continuing the administration one or more first depression treatments; administering an additional depression treatment; or administering one or more second depression treatments (switching the treatment regimen).

In one aspect, the biomarker metabolites are acylcarnitines (short-, medium-, and long-chain); amino acids; biogenic amines, glycerophospholipids, sphingolipids, tryptophan metabolites, phenylalanine metabolites, tyrosine metabolites, sulfur-amino acid metabolites, tocopherol metabolites, purine metabolites, or other metabolites.

In another aspect, the treatment is maintained or adjusted based on the concentration levels of one or more biomarker metabolites, ratios of biomarker metabolites, or combinations thereof. In another aspect, the depression is Major Depression Disorder (MDD), core depression (CD+), anxious depression (ANX+), neurovegetative symptoms of melancholia (NVSM+), or subclinical characteristics associated with depression. In another aspect, the antidepressant comprises one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram. In another aspect, the treatment further comprises administering one or more of exercise, dietary supplements, prebiotics, probiotics, probiotics, dietary changes, or an elimination diet.

Another embodiment described herein is a method of treating depression and evaluating the treatment efficacy comprising: identifying a subject suffering from or at risk of developing depression; administering an effective amount sufficient to attenuate, reduce, or eliminate the symptoms of depression of one or more antidepressants, cognitive behavior therapy, exercise, dietary supplements, prebiotics, probiotics, or dietary changes, or a combination thereof over a period of time; obtaining a sample from the subject and evaluating concentration levels of one or more biomarker metabolites; evaluating the concentration level of the one or more biomarker metabolites in comparison to control concentration levels of the one or more biomarker metabolites; evaluating the efficacy of the depression treatment; and continuing the administration one or more first depression treatments; administering an additional depression treatment; or administering one or more second depression treatments (switching the treatment regimen).

In one aspect, the biomarker metabolites are acylcarnitines (short-, medium-, and long-chain); amino acids; biogenic amines, glycerophospholipids, sphingolipids, tryptophan metabolites, phenylalanine metabolites, tyrosine metabolites, sulfur-amino acid metabolites, tocopherol metabolites, purine metabolites, or other metabolites.

In another aspect, the sample from the subject is one or more of whole blood, serum, plasma, urine, saliva, or other body fluids. In another aspect, the control sample is from an untreated subject or a subject or population of subjects not experiencing depression or not at risk for depression. In another aspect, the biomarker metabolite concentration level is greater than the control concentration level. In another aspect, the biomarker metabolite concentration level is less than the control concentration level. In another aspect, two or more biomarker metabolite concentration levels covary and are greater than the control or covary and are less than the control concentration levels (positive correlation). In another aspect, two or more biomarker metabolite concentration levels vary dissimilarly (negative correlation) compared to the control concentration levels. In another aspect, the period of time is at least: 4-weeks, 8-weeks, 16-weeks, 32-weeks, 64-weeks, or greater than 64-weeks. In another aspect, the depression is Major Depression Disorder (MDD), core depression (CD+), anxious depression (ANX+), neurovegetative symptoms of melancholia (NVSM+), or subclinical characteristics associated with depression. In another aspect, the antidepressant comprises one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram. In another aspect, the efficacy of the depression treatment is evaluated using the Hamilton Depression Rating Scale (HRSD₁₇), the Quick Inventory of Depressive Symptomatology (QIDS), subscales thereof, or specific questions thereof.

Another embodiment described herein is a method for screening a subject for depression or an increased risk of depression, the method comprising: obtaining a sample from the subject and determining concentration levels of one or more biomarker metabolites in the sample from the subject; and diagnosing the subject as having depression or an increased risk of depression when the concentration levels of the one or more biomarker metabolites in the sample from the subject is different from (greater or less) than the concentration levels the one or more biomarker metabolites in a control sample.

In one embodiment, the method further comprises: initially treating the subject for depression by administering an effective amount sufficient to attenuate, reduce, or eliminate the symptoms of depression of one or more of antidepressants, cognitive behavior therapy, exercise, dietary supplements, prebiotics, probiotics, dietary changes, or an elimination diet for a period of time.

In another embodiment, the method further comprises: obtaining an additional sample from the subject and reevaluating the concentration level of the one or more biomarker metabolites in comparison to the subject's initial concentration levels of the one or more biomarker metabolites and the control concentration levels of the one or more biomarker metabolites; evaluating the efficacy of the initial depression treatment; and continuing the one or more initial depression treatments, administering an additional depression treatment, or administering one or more second depression treatments (switching the treatment regimen).

In one aspect, the biomarker metabolites are acylcarnitines (short-, medium-, and long-chain); amino acids; biogenic amines, glycerophospholipids, sphingolipids, tryptophan metabolites, phenylalanine metabolites, tyrosine metabolites, sulfur-amino acid metabolites, tocopherol metabolites, purine metabolites, or other metabolites. In another aspect, the sample from the subject is one or more of whole blood, serum, plasma, urine, saliva, or other body fluids. In another aspect, the control sample is from an untreated subject or a subject or population of subjects not experiencing depression or not at risk for depression. In another aspect, the biomarker metabolite concentration level increases compared to the initial sample or control. In another aspect, the biomarker metabolite concentration level decreases compared to the initial sample or control. In another aspect, two or more biomarker metabolite concentration levels covary and increase or covary and decrease (positive correlation) compared to the initial sample or control. In another aspect, two or more biomarker metabolite concentration levels vary dissimilarly (negative correlation) the initial sample or control. In another aspect, the control sample is from an untreated subject or a subject or population of subjects not experiencing depression or not at risk for depression.

In another aspect, the depression is Major Depression Disorder (MDD), core depression (CD+), anxious depression (ANX+), neurovegetative symptoms of melancholia (NVSM+), or subclinical characteristics associated with depression. In another aspect, the period of time is at least: 4-weeks, 8-weeks, 12-weeks, 16-weeks, 32-weeks, 64-weeks, or greater than 64-weeks.

In another aspect, the antidepressant comprises one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram. In another aspect, the depression and efficacy of treatment is evaluated using the Hamilton Depression Rating Scale (HRSD₁₇), the Quick Inventory of Depressive Symptomatology (QIDS), subscales thereof, or specific questions thereof. In another aspect, if the subject's depression score statistically significantly decreases from the initial score, the treatment is efficacious.

In another aspect, the method further comprises detecting at least one positively correlated biomarker metabolite, wherein detecting the at least one positively correlated biomarker metabolite is associated with a presence of at least one independent indicator of depression.

In another aspect, the positively correlated biomarker metabolite is one or more of acylcarnitine C3, acylcarnitine C5, α-aminoadipic acid, sarcosine, serotonin (5HT), or 3-methoxy-4-hydroxyphenylglycol (MHPG), or combinations thereof.

In another aspect, at least one independent indicator of depression comprises a HRSD₁₇ score of >18.

In another aspect the method further comprises detecting at least one negatively correlated biomarker metabolite, wherein detecting the at least one negatively correlated biomarker metabolite is associated with an absence of at least one independent indicator of depression.

In another aspect, the baseline detection of an increased level of at least one biomarker metabolite compared to a control comprising one or more of acylcarnitine C3, acylcarnitine C5, α-aminoadipic acid, sarcosine, serotonin or 3-methoxy-4-hydroxyphenylglycol (MHPG), or combinations thereof indicates the subject has at least one independent indicator of depression.

Another embodiment described herein is a method for predicting a depression subject's propensity of treatment success (depression remission) in response to an antidepressant, the method comprising: obtaining a sample from the subject and evaluating the concentration level of the one or more biomarker metabolites in comparison to the control concentration levels of the one or more biomarker metabolites; administering an effective amount sufficient to attenuate, reduce, or eliminate the symptoms of depression of one or more antidepressants, cognitive behaviorial therapy, or a combination thereof for a period of time; obtaining an additional sample from the subject and evaluating the concentration level of the one or more biomarker metabolites in comparison to the subject's initial concentration levels of the one or more biomarker metabolites and the control concentration levels of the one or more biomarker metabolites; continuing the one or more initial depression treatments, administering an additional depression treatment, or administering one or more second depression treatments (switching the treatment regimen).

In one aspect, the biomarker metabolites are acylcarnitines (short-, medium-, and long-chain); amino acids; biogenic amines, glycerophospholipids, sphingolipids, tryptophan metabolites, phenylalanine metabolites, tyrosine metabolites, sulfur-amino acid metabolites, tocopherol metabolites, purine metabolites, or other metabolites. In another aspect, the sample from the subject is one or more of whole blood, serum, plasma, urine, saliva, or other body fluids. In another aspect, the control sample is from an untreated subject or a subject or population of subjects not experiencing depression or not at risk for depression. In another aspect, the biomarker metabolite concentration level increases compared to the initial sample or control. In another aspect, the biomarker metabolite concentration level decreases compared to the initial sample or control. In another aspect, two or more biomarker metabolite concentration levels covary and increase or covary and decrease (positive correlation) compared to the initial sample or control. In another aspect, two or more biomarker metabolite concentration levels vary dissimilarly (negative correlation) the initial sample or control. In another aspect, the control sample is from an untreated subject or a subject or population of subjects not experiencing depression or not at risk for depression.

In another aspect, the depression is Major Depression Disorder (MDD), core depression (CD+), anxious depression (ANX+), neurovegetative symptoms of melancholia (NVSM+), or subclinical characteristics associated with depression. In another aspect, the period of time is at least: 4-weeks, 8-weeks, 12-weeks, 16-weeks, 32-weeks, 64-weeks, or greater than 64-weeks.

In another aspect, the antidepressant comprises one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is a selective serotonin reuptake inhibitor (SSRI). In another aspect, the antidepressant comprises escitalopram, citalopram, fluoxetine, sertraline, paroxetine, fluvoxamine, vilazodone, vortioxetine, or duloxetine. In another aspect, wherein the antidepressant comprises escitalopram or citalopram. In another aspect, the depression and efficacy of treatment is evaluated using the Hamilton Depression Rating Scale (HRSD₁₇), the Quick Inventory of Depressive Symptomatology (QIDS), subscales thereof, or specific questions thereof.

In another aspect, if the subject has greater baseline concentration levels of acylcarnitines C3, C5, α-aminoadipic acid, sarcosine, or serotonin in comparison to control concentration levels, and after at least 8-weeks of treatment these biomarker metabolites had concentrations greater than control levels, the subject has a propensity of antidepressant treatment success.

In another aspect, if the subject has greater baseline concentration levels of acylcarnitines C3, C5, α-aminoadipic acid, sarcosine, or serotonin in comparison to control concentration levels, and the concentration levels of these biomarker metabolites remains greater than baseline after 8 weeks of antidepressant treatment, the subject has a propensity of antidepressant treatment success.

In another aspect, if the subject has greater baseline concentration levels of one or more of 3-methoxy-4-hydroxyphenylglycol (MHPG) and serotonin (5HT) compared to control concentration levels and after at least 8-weeks of antidepressant treatment, the levels of MHPG and 5HT decreased compared to the baseline concentration levels, the subject has a propensity of antidepressant treatment success.

In another aspect, if the subject has greater concentrations levels of one or more of PC aa C34:1, PC aa C34:2, PC aa C36:2 and PC aa C36:4 compared to baseline concentration levels after at least 8-weeks of antidepressant treatment, the subject has a propensity of antidepressant treatment success.

In another aspect, if the subject has greater concentration levels after at least 8-weeks of antidepressant treatment of one or more of acylcarnitine (C5-M-DC), histidine, proline, kynurenine and trans-4-hydroxyproline, PC aa C34:1, PC aa C34:2, PC aa C36:1, PC aa C36:2, PC aa C36:3; PC aa C36:4, PC aa C40:1, PC ae C34:2, PC ae C34:3, PC ae C36:1, PC ae C36:2, PC ae C36:3, PC ae C38:2, or PC ae C38:3, and the concentration levels of one or more of these biomarker metabolites are inversely associated with changes in the subject's depression score, the subject has a propensity of antidepressant treatment success.

Another embodiment described herein is a method for evaluating a depression subject's response to antidepressant treatment, cognitive behavioral therapy or a combination thereof, the method comprising: obtaining a sample from the subject and evaluating the concentration level of the one or more biomarker metabolites in comparison to the control concentration levels of the one or more biomarker metabolites; administering an effective amount sufficient to attenuate, reduce, or eliminate the symptoms of depression of one or more antidepressants, cognitive behaviorial therapy, or a combination thereof for a period of time; obtaining an additional sample from the subject and evaluating the concentration level of the one or more biomarker metabolites in comparison to the subject's initial concentration levels of the one or more biomarker metabolites and the control concentration levels of the one or more biomarker metabolites; evaluating the efficacy of the initial depression treatment; and continuing the one or more initial depression treatments, administering an additional depression treatment, or administering one or more second depression treatments (switching the treatment regimen).

In one aspect, the biomarker metabolites are acylcarnitines (short-, medium-, and long-chain); amino acids; biogenic amines, glycerophospholipids, sphingolipids, tryptophan metabolites, phenylalanine metabolites, tyrosine metabolites, sulfur-amino acid metabolites, tocopherol metabolites, purine metabolites, or other metabolites. In another aspect, the sample from the subject is one or more of whole blood, serum, plasma, urine, saliva, or other body fluids. In another aspect, the control sample is from an untreated subject or a subject or population of subjects not experiencing depression or not at risk for depression. In another aspect, the biomarker metabolite concentration level increases compared to the initial sample or control. In another aspect, the biomarker metabolite concentration level decreases compared to the initial sample or control. In another aspect, two or more biomarker metabolite concentration levels covary and increase or covary and decrease (positive correlation) compared to the initial sample or control. In another aspect, two or more biomarker metabolite concentration levels vary dissimilarly (negative correlation) the initial sample or control. In another aspect, the control sample is from an untreated subject or a subject or population of subjects not experiencing depression or not at risk for depression.

In another aspect, the depression is Major Depression Disorder (MDD), core depression (CD+), anxious depression (ANX+), neurovegetative symptoms of melancholia (NVSM+), or subclinical characteristics associated with depression. In another aspect, the period of time is at least: 4-weeks, 8-weeks, 12-weeks, 16-weeks, 32-weeks, 64-weeks, or greater than 64-weeks.

In another aspect, the antidepressant comprises one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram. In another aspect, the depression and efficacy of treatment is evaluated using the Hamilton Depression Rating Scale (HRSD₁₇), the Quick Inventory of Depressive Symptomatology (QIDS), subscales thereof, or specific questions thereof. In another aspect, if the subject's depression score statistically significantly decreases from the initial score, the treatment is efficacious.

In another aspect, after at least 8-weeks of antidepressant treatment, the subject has: greater concentration levels compared to baseline concentration levels of acylcarnitine C3, C4 and C5; arginine, proline, methionine sulfoxide; phosphatidylcholines (PC aa C36:1, C30:0, C42:2); phosphatidylcholine ethers (PC ae C34:3, C38:2, C36:3); and sphingomyelin SM C24:0; and decreased concentration levels compared to baseline of acylcarnitines C8, C10, C12, C14:2, C16, C16:1, C18, C18:1, and C18:2; serotonin and sarcosine.

In another aspect, after at least 8-weeks of antidepressant treatment, the subject has: greater concentration levels compared to baseline levels of acylcarnitine indoles in TRP metabolism; 5-hydroxytryptophan (THTP), 5-hydroxyindoleacetic acid (5HIAA), indole-3-acetic acid (I3AA); homogentisic acid (HGA), vanillylmandelic acid, 4-hydroxyphenylacetic acid (4HPAC), 4-hydroxybenzoic acid (4HBAC), guanine (G), paraxanthine (PXAN), xanthosine (XANTH), salicylic acid (SA), the ratio of 4-hydroxyphenylacetic acid to tyrosine, the ratio of 5-hydroxyindoleacetic acid to serotonin, the ratio of uric acid to xanthosine, and the ratio of paraxanthine to xanthosine; and decreased concentration levels of serotonin, 3-methoxy-4-hydroxyphenylethyleneglycol (MHPG), xanthosine, hypoxanthine, and the ratio of 3-methoxy-4-hydroxyphenylethyleneglycol to tyrosine.

In another aspect, after at least 8-weeks of cognitive behavioral therapy, the subject has: greater concentration levels compared to baseline levels of acylcarnitine (C0, C3, C4, and C5), α-aminoadipic acid, glutamate, and proline, isoleucine, aspartic acid, histidine, valine, tryptophan, tyrosine, phenylalanine, methionine, methionine-sulfoxide, and sarcosine; acylcarnitines (C10:2, C161-OH, C16-OH, C3:1, C3-OH, C4:1, C5:1, C5:1-DC, C5-OH (C3-DC-M), C6:1, C9); Phosphatidylcholines: (PCaa C24:0, C28:1, C30:0, C32:0, C32:1, C32:3, C34:1, C34:2, C34:3, C34:4, C36:1, C36:2, C36:3, C36:4, C36:5, C36:6, C38:0, C38:3, C38:4, C38:5, C38:6, C40:2, C40:4, C40:5, C40:6, C42:0, C42:1, C42:2, C42:4, C42:5, C42:6); (PC ae C30:2, C32:1, C32:2, C34:0, C34:1, C34:2, C34:3, C36:0, C36:1, C36:2, C36:3, C36:4, C36:5, C38:0, C38:2, C38:3, C38:4, C38:5, C38:6, C40:1, C40:2, C40:3, C40:4, C40:5, C40:6, C42:1, C42:2, C42:3, C42:4, C42:5, C44:3, C44:4, C44:5, C44:6); (LysoPC a C24:0, C26:0, C26:1, C28:0, C28:1); Sphingomyelins: SM (OH) C14:1, C22:1, C24:1, SM C16:0, C16:1, C18:0, C24:0, C24:1, C26:0, C26:1; and decreases in the subject's depression score.

Another embodiment described herein is a method for differentiating a depression subject's depression phenotype, the method comprising: identifying a subject experiencing depression or at risk of depression; obtaining a sample from the subject and evaluating the concentration level of the one or more biomarker metabolites in comparison to the control concentration levels of the one or more biomarker metabolites; administering an effective amount sufficient to attenuate, reduce, or eliminate the symptoms of depression of one or more antidepressant antidepressants, cognitive behaviorial therapy, or a combination thereof for a period of time; obtaining an additional sample from the subject and evaluating the concentration level of the one or more biomarker metabolites in comparison to the subject's initial concentration levels of the one or more biomarker metabolites and the control concentration levels of the one or more biomarker metabolites; evaluating the phenotype and efficacy of the initial depression treatment; and continuing the one or more initial depression treatments, administering an additional depression treatment, or administering one or more second depression treatments (switching the treatment regimen).

In one aspect, the biomarker metabolites are acylcarnitines (short-, medium-, and long-chain); amino acids; biogenic amines, glycerophospholipids, sphingolipids, tryptophan metabolites, phenylalanine metabolites, tyrosine metabolites, sulfur-amino acid metabolites, tocopherol metabolites, purine metabolites, or other metabolites. In another aspect, the sample from the subject is one or more of whole blood, serum, plasma, urine, saliva, or other body fluids. In another aspect, the control sample is from an untreated subject or a subject or population of subjects not experiencing depression or not at risk for depression. In another aspect, the biomarker metabolite concentration level increases compared to the initial sample or control. In another aspect, the biomarker metabolite concentration level decreases compared to the initial sample or control. In another aspect, two or more biomarker metabolite concentration levels covary and increase or covary and decrease (positive correlation) compared to the initial sample or control. In another aspect, two or more biomarker metabolite concentration levels vary dissimilarly (negative correlation) the initial sample or control. In another aspect, the control sample is from an untreated subject or a subject or population of subjects not experiencing depression or not at risk for depression.

In another aspect, the depression is Major Depression Disorder (MDD), core depression (CD+), anxious depression (ANX+), neurovegetative symptoms of melancholia (NVSM+), or subclinical characteristics associated with depression. In another aspect, the period of time is at least: 4-weeks, 8-weeks, 12-weeks, 16-weeks, 32-weeks, 64-weeks, or greater than 64-weeks.

In another aspect, the antidepressant comprises one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In another aspect, the antidepressant is one or more selective serotonin reuptake inhibitors (SSRI). In another aspect, the antidepressant comprises escitalopram, citalopram, fluoxetine, sertraline, paroxetine, fluvoxamine, vilazodone, vortioxetine, or duloxetine. In another aspect, the antidepressant comprises escitalopram or citalopram. In another aspect, the type of depression (phenotype) and efficacy of the depression treatment is evaluated using the Hamilton Depression Rating Scale (HRSD₁₇), the Quick Inventory of Depressive Symptomatology (QIDS), subscales thereof, or specific questions thereof. In another aspect, if the subject's depression score statistically significantly decreases from the initial score, the treatment is efficacious.

In another aspect, the baseline detection of an decreased concentration level of at least one biomarker metabolite compared to a control comprising one or more of acylcarnitines C0, C3, C18m, C5:1, C5-DC/C6-OH, C16-OH, or combinations thereof indicates the subject has at least one independent indicator of core depression (CD+).

In another aspect, the baseline detection of an decreased concentration level of at least one biomarker metabolite compared to a control comprising one or more of acylcarnitines C5-DC/C6-OH and C16-OH, or combinations thereof and higher concentration levels of acylcarnitine C10 compared to a control, indicates the subject has at least one independent indicator of neurovegetative symptoms of melancholia (NVSM+).

In another aspect, after at least 8-weeks of antidepressant treatment, subjects having core depression (CD+), had greater concentration levels of acylcarnitines C5-DC/C6-OH, C5-M-DC, C6, C14:1, C16, C16-OH, C16:1, and C18:1-OH compared with concentration levels of subjects having neurovegetative symptoms of melancholia (NVSM+).

In another aspect, after at least 8-weeks of antidepressant treatment, subjects having core depression (CD+), had lower concentration levels of acylcarnitines C2, C3 and C6 compared with anxious depression (ANX+) subjects.

In another aspect, after at least 8-weeks of antidepressant treatment, subjects having neurovegetative symptoms of melancholia (NVSM+), had lower concentration levels of acylcarnitines C0, C2, C3, C5-DC/C6-OH, C5-M-DC, C6, C10, C14:1, C16, C16-OH, C16:1 and C18:1-OH compared with anxious depression (ANX+) subjects.

In another aspect, after at least 8-weeks of antidepressant treatment, subjects having core depression (CD+), had increased concentration levels of acylcarnitines C0, C3, C4, C5-M-DC and C5:1 compared with baseline concentration levels of these biomarker metabolites.

In another aspect, after at least 8-weeks of antidepressant treatment, subjects having core depression (CD+) or anxious depression (ANX+), had increased concentration levels of acylcarnitines C0, C3, and C4 compared with baseline concentration levels of these biomarker metabolites.

In another aspect, after at least 8-weeks of antidepressant treatment, subjects having core depression (CD+) or neurovegetative symptoms of melancholia (NVSM+), had decreased concentration levels of acylcarnitines C8, C10, and C12 compared with baseline concentration levels of these biomarker metabolites.

In another aspect, after at least 8-weeks of antidepressant treatment, subjects having core depression (CD+) or neurovegetative symptoms of melancholia (NVSM+), had decreased concentration levels of acylcarnitines C14:1, C14:2, C16, C16-OH, C16:1, C18, C18:1, C18:1-OH, C18:2 compared with baseline concentration levels of these biomarker metabolites.

In another aspect, after at least 8-weeks of antidepressant treatment, subjects having core depression (CD+), neurovegetative symptoms of melancholia (NVSM+) or anxious depression (ANX+), had decreased concentration levels of acylcarnitines, C5-OH, C16:1, C18:1, and C18:2, compared with baseline concentration levels of these biomarker metabolites.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

### Study Design and Participants:

The design and clinical outcomes of the PGRN-AMPS study have been previously published (Mrazek et al., J. Clin. Psychopharmacol. 34, 313-317 (2014)). The trial enrolled 800 MDD patients between 18-84 years of age from Mayo Clinic psychiatry or primary care clinics. Patients received open-label treatment with either citalopram (20-40 mg/day) or escitalopram (10-20 mg/day) for 8 weeks. Severity of MDD was assessed using the Hamilton Depression Rating Scale, 17-item (HRSD₁₇) (Hamilton, Br. J. Soc. Clin. Psychol. 6(4): 278-96 (1967)), at baseline, week 4 and week 8. The PGRN-AMPS protocol was approved by Institutional Review Board of Mayo Clinic.

Metabolomic Profiling Using Absolute IDQ p180 Kit

Metabolites were measured with a targeted metabolomics approach using the AbsoluteIDQ® p180 Kit (BIOCRATES Life Science AG, Innsbruck, Austria), with a ultra-performance liquid chromatography (UPLC)/MS/MS system (Acquity UPLC (Waters), TQ-S triple quadrupole MS/MS (Waters)), which provides measurements of up to 186 endogenous metabolites quantitatively (amino acids and biogenic amines) and semi-quantitatively (acylcarnitines, sphingomyelins, phosphatidylcholines and lysophosphatidylcholines across multiple classes). The AbsoluteIDQ® p180 kit has been fully validated according to European Medicine Agency Guidelines on bioanalytical method validation. Additionally, the kit plates include an automated technical validation to assure the validity of the run and provide verification of the actual performance of the applied quantitative procedure including instrumental analysis. The technical validation of each analyzed kit plate was performed using MetIDQ® software based on results obtained and defined acceptance criteria for blank, zero samples, calibration standards and curves, low/medium/high-level QC samples and measured signal intensity of internal standards over the plate. This platform has been used in hundreds of studies, including prior investigations of MDD. De-identified samples were analyzed following the manufacturer's protocol, with metabolomics labs blinded to clinical data.

### Quality Control of P180 Profiles

Metabolites with >40% of measurements below the lower limit of detection (LOD) were excluded from the analysis. Each assay plate included a set of duplicates obtained by combining approximately 10 μL from the first 76 samples in the study (QC pool duplicates) to allow appropriate inter-plate abundance scaling based specifically on this cohort of samples. To adjust for the batch effects, a correction factor for each metabolite in a specific plate was obtained by dividing metabolite's SPQC global average by SPQC average within the plate. <LOD values were imputed using each metabolite's LOD/2 value followed by log 2 transformation.

We checked for the presence of multivariate outlier samples by evaluating the squared Mahalanobis distance of samples. Samples with Mahalanobis distances exceeding the critical value corresponding to Bonferroni-corrected threshold (0.05/n, n: number of samples) of the Chi-square distribution with m degrees of freedom (m=163: number of metabolite) were flagged as outlier. This procedure identified 15 outlier samples. This resulted in an analysis data set containing 269 subjects, 537 samples and 163 metabolites of baseline (n=240), week 4 (n=144) and week 8 (n=127) samples.

### Clinical Outcomes

The HRSD₁₇ total score was used as the outcome measure for both continuous depression severity change and for defining categorical outcomes. Consistent with prior definitions, patient outcomes at week 8 were categorized as “remission” (HRSD_{17≤}7); “response without remission” (250% reduction from baseline HRSD₁₇, but not reaching remission threshold); “partial response” (30-49% reduction from baseline HRSD₁₇ score); and “treatment failure” (<30% reduction from baseline HRSD₁₇ score). Rush et al., Neuropsychopharmacol. 31(9): 1841-1853 (2006); Dunlop, Kelley et al., Am. J. Psychiatry 174, 546-556 (2017).

### Statistical Analysis

Differences in demographic variables and depression scores across the response groups were evaluated using ANOVA and the Pearson Chi-squared test (for categorical variables). All association and differential abundance analyses were performed in a metabolite-wise manner. To examine the significance of log 2-fold change in metabolite concentrations, linear mixed effect models (with random intercept) with log 2 metabolite levels as the dependent variable were fitted while correcting for age, sex, baseline HRSD₁₇ and specific antidepressant (citalopram/escitalopram). Then we used the “emmeans” R package to compute the least squared means of the contrasts of interest (week 8 vs. baseline) and their corresponding p-values. Adjustments for multiple comparisons were made using the Benjamini-Hochberg procedure to control the false discovery rate. We investigated the global correlation structure of changing metabolites from baseline to week 8 using Spearman's ranked correlation to identify biochemically related metabolites on which SSRI exposure has similar effect, followed by hierarchical clustering to group similar correlated metabolites.

To detect whether changes in metabolites were associated with clinical outcome, we conducted continuous and categorical analyses. In the continuous analysis, the associations of changes in HRSD₁₇ score after 8 weeks with changes in metabolite levels were tested using linear regression models corrected for age and sex. In the categorical analysis, profiles of remission (“remitters” vs. “response” vs. “partial response” vs. “treatment failure”) were compared using linear mixed effect models (with random intercept), with log 2 metabolite levels as the dependent variable and the interaction of the week 8 outcome (4 level categorical variable: remitters; response without remission; partial response; treatment failures) and visit (2 level categorical variable: baseline; week 8) as independent variables while correcting for age, sex, HRSD₁₇ score and antidepressant (citalopram/escitalopram), as the method used by other investigators to maximize the ability to identify biological characteristics most clearly associated with differential outcomes (Dunlop et al., Am. J. Psychiatry 174, 546-556 (2017); Vadodaria et al, Mol. Psychiatry doi:10.1038/s41380-019-0363-y (2019)). Then the fitted models were used to conduct contrasts between the “remission” and “treatment failure” groups at baseline and week 8 using the “emmeans” R-package.

### Results

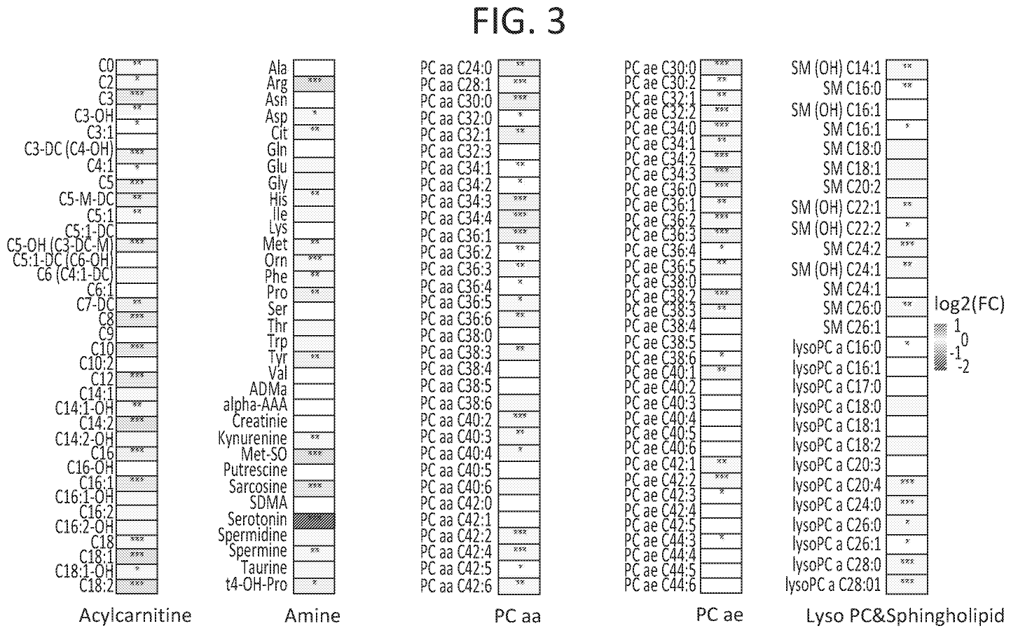

Table 1 depicts demographics of Mayo-PGRN study at baseline. Age, sex and body mass index were not significantly different across response groups. Table 2 and FIG. 3 depict the metabolites measured in the p180 kit.

TABLE 1
Demographics of Mayo PGRN study
Baseline

| | | | | Partial- | Non- | | |
|---|---|---|---|---|---|---|---|
| | | Remission | Response | Response | Response | Combined | |
| | N | (N = 122) | (N = 48) | (N = 45) | (N = 25) | (N = 240) | p-value |
| Age (years), | 240 | 40.18 | 38.20 | 40.31 | 41.83 | 39.98 | 0.86 |
| Mean (SD) | | (13.36) | (11.49) | (11.39) | (17.09) | (13.06) | |
| Sex: Male | 240 | 32% (39) | 42% (20) | 29% (13) | 36% (9) | 34% (81) | 0.56 |
| BMI (Kg/M2), | 192 | 33.59 | 27.68 | 29.84 | 28.56 | 31.15 | 0.42 |
| Mean (SD) | | (26.35) | (6.51) | (7.67) | (7.23) | (19.41) | |
| HRSD₁₇ | 240 | 21.25 | 24.79 | 22.16 | 22.12 | 22.22 | P <0.001 |
| Mean (SD) | | (5.04) | (4.47) | (4.48) | (4.13) | (4.90) | |

Remission: HRSD₁₇ at week 8< = 7; Response: >50% improvement in HRSD₁₇ but not remission;
Partial-Response: >30% and <50% improvement in HR5D₁₇; Non-Response: <30% improvement in
HR5D₁₇. BMI: Body Mass Index. HRSD₁₇: The Hamilton Depression Rating Scale-17 items.

TABLE 2
Metabolites measured in the p180 kit

| | | | Coefficient of | |
|---|---|---|---|---|
| | | | Variation-SPQC | % < Limit of |
| Class | Abbreviation | Metabolite Name | Samples | Detection |
| Short-chain | C0 | Carnitine | 4.60 | 0.00 |
| Acylcarnitines | C2 | Acetylcarnitine | 4.25 | 0.00 |
| | C3 | Propionylcarnitine | 4.86 | 0.00 |
| | C3—OH | Hydroxypropionylcarnitine | 10.22 | 0.35 |
| | C3:1 | Propenoylcarnitine | 10.31 | 0.17 |
| | C3-DC (C4—OH) | Hydroxybutyrylcarnitine | 7.65 | 0.17 |
| | C4 | Butyrylcarnitine | 4.32 | 0.00 |
| | C4:1 | Butenylcarnitine | 10.86 | 1.04 |
| | C5 | Valerylcarnitine | 5.93 | 0.00 |
| | C5-M-DC | Methylglutarylcarnitine | 8.82 | 21.80 |
| | C5:1 | Tiglylcarnitine | 7.80 | 0.52 |
| | C5:1-DC | Glutaconylcarnitine | 17.06 | 0.35 |
| | C5—OH (C3-DC-M) | Hydroxyvalerylcarnitine | 7.61 | 0.00 |
| | | (Methylmalonylcarnitine) | | |
| | C5-DC (C6—OH) | Glutarylcarnitine* | 13.51 | 0.17 |
| | | (Hydroxyhexanoylcarnitine) | | |
| Medium-chain | C6 (C4:1-DC) | Hexanoylcarnitine | 5.56 | 0.35 |
| Acylcarnitines | | (Fumarylcarnitine) | | |
| | C6:1 | Hexenoylcarnitine | 9.66 | 0.52 |
| | C7-DC | Pimelylcarnitine | 12.80 | 1.38 |
| | C8 | Octanoylcarnitine | 4.67 | 21.80 |
| | C9 | Nonaylcarnitine | 7.58 | 0.00 |
| | C10 | Decanoylcarnitine | 4.17 | 1.21 |
| | C10:1 | Decenoylcarnitine | NA | 100.00 |
| | C10:2 | Decadienylcarnitine | 6.39 | 9.34 |
| | C12 | Dodecanoylcarnitine | 4.58 | 22.49 |
| | C12-DC | Dodecanedioylcarnitine | NA | 100.00 |
| | C12:1 | Dodecenoylcarnitine | NA | 100.00 |
| Long-chain | C14 | Tetradecanoylcarnitine | 4.08 | 45.16 |
| Acylcarnitines | C14:1 | Tetradecenoylcarnitine | 14.83 | 0.00 |
| | C14:1-OH | Hydroxytetradecenoylcarnitine | 8.84 | 1.04 |
| | C14:2 | Tetradecadienylcarnitine | 4.85 | 4.50 |
| | C14:2-OH | Hydroxytetradecadienylcarnitine | 9.63 | 2.42 |
| | C16 | Hexadecanoylcarnitine | 4.62 | 0.00 |
| | C16—OH | Hydroxyhexadecanoylcarnitine | 10.17 | 8.48 |
| | C16:1 | Hexadecenoylcarnitine | 5.66 | 0.35 |
| | C16:1-OH | Hydroxyhexadecenoylcarnitine | 7.11 | 7.79 |
| | C16:2 | Hexadecadienylcarnitine | 5.75 | 23.70 |
| | C16:2-OH | Hydroxyhexadecadienylcarnitine | 6.28 | 11.76 |
| | C18 | Octadecanoylcarnitine | 4.94 | 0.00 |
| | C18:1 | Octadecenoylcarnitine | 4.47 | 0.00 |
| | C18:1-OH | Hydroxyoctadecenoylcarnitine | 10.44 | 5.88 |
| | C18:2 | Octadecadienylcarnitine | 3.71 | 0.00 |
| Amino Acids | Alanine | | 4.69 | 0.00 |
| | Arginine | | 6.74 | 0.00 |
| | Asparagine | | 5.41 | 0.00 |
| | Aspartate | | 22.31 | 1.73 |
| | Citrulline | | 6.32 | 0.00 |
| | Glutamine | | 5.35 | 0.00 |
| | Glutamate | | 7.44 | 0.00 |
| | Glycine | | 5.19 | 0.00 |
| | Histidine | | 4.34 | 0.00 |
| | Isoleucine | | 6.25 | 0.00 |
| | Lysine | | 14.93 | 0.00 |
| | Methionine | | 9.40 | 0.00 |
| | Ornithine | | 10.89 | 0.00 |
| | Phenylalanine | | 3.93 | 0.00 |
| | Proline | | 3.64 | 0.00 |
| | Serine | | 7.35 | 0.00 |
| | Threonine | | 4.49 | 0.00 |
| | Tryptophan | | 4.06 | 0.00 |
| | Tyrosine | | 4.33 | 0.00 |
| | Valine | | 6.52 | 0.00 |
| Biogenic Amines | Acetylornithine | | NA | 100.00 |
| | Asymmetric | | 15.94 | 0.17 |
| | dimethylarginine | | | |
| | alpha-Aminoadipic | | 19.10 | 10.73 |
| | acid | | | |
| | Carnosine | | NA | 99.65 |
| | Creatinine | | 3.72 | 0.00 |
| | DOPA | | 7.83 | 51.56 |
| | Dopamine | | NA | 100.00 |
| | Histamine | | NA | 99.83 |
| | Kynurenine | | 5.72 | 0.17 |
| | Methionine | | 16.78 | 3.63 |
| | sulfoxide | | | |
| | Nitrotyrosine | | NA | 100.00 |
| | Phenylethylamine | | NA | 100.00 |
| | Putrescine | | 14.08 | 1.73 |
| | Sarcosine | | 6.43 | 0.00 |
| | Symmetric | | 7.28 | 0.00 |
| | dimethylarginine | | | |
| | Serotonin | | 9.52 | 38.93 |
| | Spermidine | | 9.55 | 0.17 |
| | Spermine | | 16.72 | 2.08 |
| | Taurine | | 3.93 | 0.00 |
| | cis-4- | | NA | 99.83 |
| | hydroxyproline | | | |
| | trans-4- | | 4.04 | 0.00 |
| | hydroxyproline | | | |
| Glycerophospholipids- PC aa | PC aa C24:0 | | 19.87 | 0.87 |
| | PC aa C26:0 | | NA | 99.83 |
| | PC aa C28:1 | | 3.99 | 0.00 |
| | PC aa C30:0 | | 4.11 | 0.00 |
| | PC aa C32:0 | | 4.33 | 0.00 |
| | PC aa C32:1 | | 5.27 | 0.00 |
| | PC aa C32:3 | | 4.98 | 0.00 |
| | PC aa C34:1 | | 8.98 | 0.00 |
| | PC aa C34:2 | | 9.30 | 0.00 |
| | PC aa C34:3 | | 4.93 | 0.00 |
| | PC aa C34:4 | | 5.03 | 0.00 |
| | PC aa C36:0 | | NA | 75.78 |
| | PC aa C36:1 | | 4.28 | 0.00 |
| | PC aa C36:2 | | 9.23 | 0.00 |
| | PC aa C36:3 | | 8.59 | 0.00 |
| | PC aa C36:4 | | 8.83 | 0.00 |
| | PC aa C36:5 | | 4.85 | 0.00 |
| | PC aa C36:6 | | 4.89 | 0.00 |
| | PC aa C38:0 | | 4.28 | 0.00 |
| | PC aa C38:3 | | 4.39 | 0.00 |
| | PC aa C38:4 | | 7.41 | 0.00 |
| | PC aa C38:5 | | 4.51 | 0.00 |
| | PC aa C38:6 | | 4.43 | 0.00 |
| | PC aa C40:1 | | 1.98 | 51.73 |
| | PC aa C40:2 | | 5.72 | 0.00 |
| | PC aa C40:3 | | 4.93 | 0.00 |
| | PC aa C40:4 | | 4.21 | 0.00 |
| | PC aa C40:5 | | 4.59 | 0.00 |
| | PC aa C40:6 | | 4.49 | 0.00 |
| | PC aa C42:0 | | 3.57 | 0.00 |
| | PC aa C42:1 | | 4.11 | 0.00 |
| | PC aa C42:2 | | 3.90 | 0.17 |
| | PC aa C42:4 | | 4.59 | 0.00 |
| | PC aa C42:5 | | 4.12 | 0.00 |
| | PC aa C42:6 | | 4.11 | 0.17 |
| Glycerophospholipids- PC ae | PC ae C30:0 | | 3.63 | 0.17 |
| | PC ae C30:1 | | 29.89 | 41.52 |
| | PC ae C30:2 | | 4.54 | 0.00 |
| | PC ae C32:1 | | 4.89 | 0.00 |
| | PC ae C32:2 | | 4.89 | 0.00 |
| | PC ae C34:0 | | 4.53 | 0.00 |
| | PC ae C34:1 | | 4.40 | 0.00 |
| | PC ae C34:2 | | 4.56 | 0.00 |
| | PC ae C34:3 | | 4.54 | 0.00 |
| | PC ae C36:0 | | 5.26 | 0.17 |
| | PC ae C36:1 | | 4.64 | 0.00 |
| | PC ae C36:2 | | 4.64 | 0.00 |
| | PC ae C36:3 | | 4.51 | 0.00 |
| | PC ae C36:4 | | 4.91 | 0.00 |
| | PC ae C36:5 | | 4.62 | 0.00 |
| | PC ae C38:0 | | 3.71 | 0.00 |
| | PC ae C38:1 | | 56.50 | 61.59 |
| | PC ae C38:2 | | 8.03 | 0.00 |
| | PC ae C38:3 | | 3.77 | 0.00 |
| | PC ae C38:4 | | 4.61 | 0.00 |
| | PC ae C38:5 | | 4.14 | 0.00 |
| | PC ae C38:6 | | 4.37 | 0.00 |
| | PC ae C40:1 | | 6.33 | 0.00 |
| | PC ae C40:2 | | 3.95 | 0.00 |
| | PC ae C40:3 | | 3.93 | 0.00 |
| | PC ae C40:4 | | 3.97 | 0.00 |
| | PC ae C40:5 | | 4.07 | 0.00 |
| | PC ae C40:6 | | 4.22 | 0.00 |
| | PC ae C42:0 | | NA | 98.44 |
| | PC ae C42:1 | | 4.29 | 0.17 |
| | PC ae C42:2 | | 3.26 | 0.00 |
| | PC ae C42:3 | | 4.03 | 0.00 |
| | PC ae C42:4 | | 3.91 | 0.17 |
| | PC ae C42:5 | | 3.95 | 0.17 |
| | PC ae C44:3 | | 6.20 | 0.17 |
| | PC ae C44:4 | | 4.70 | 0.17 |
| | PC ae C44:5 | | 3.94 | 0.00 |
| | PC ae C44:6 | | 3.78 | 0.00 |
| Glycerophospholipids- Lys PC | lysoPC a C14:0 | | NA | 100.00 |
| | lysoPC a C16:0 | | 4.39 | 0.00 |
| | lysoPC a C16:1 | | 3.80 | 0.00 |
| | lysoPC a C17:0 | | 4.24 | 0.00 |
| | lysoPC a C18:0 | | 4.44 | 0.00 |
| | lysoPC a C18:1 | | 4.30 | 0.00 |
| | lysoPC a C18:2 | | 4.96 | 0.00 |
| | lysoPC a C20:3 | | 5.06 | 0.00 |
| | lysoPC a C20:4 | | 4.92 | 0.00 |
| | lysoPC a C24:0 | | 9.13 | 0.00 |
| | lysoPC a C26:0 | | 21.15 | 0.00 |
| | lysoPC a C26:1 | | 17.84 | 0.00 |
| | lysoPC a C28:0 | | 12.56 | 0.17 |
| | lysoPC a C28:1 | | 9.00 | 0.00 |
| Sphingolipids | SM (OH) C14:1 | | 7.00 | 0.00 |
| | SM C16:0 | | 7.30 | 0.00 |
| | SM (OH) C16:1 | | 7.00 | 0.00 |
| | SM C16:1 | | 6.61 | 0.00 |
| | SM C18:0 | | 7.42 | 0.00 |
| | SM C18:1 | | 7.05 | 0.00 |
| | SM C20:2 | | 40.60 | 0.00 |
| | SM (OH) C22:1 | | 9.02 | 0.00 |
| | SM (OH) C22:2 | | 7.31 | 0.00 |
| | SM C24:0 | | 10.07 | 0.00 |
| | SM (OH) C24:1 | | 9.82 | 0.00 |
| | SM C24:1 | | 8.42 | 0.00 |
| | SM C26:0 | | 11.56 | 0.00 |
| | SM C26:1 | | 9.12 | 0.00 |

Abbreviations:
SM: sphingomyelin, PC: Phosphatidylcholine, LysoPC: Lyso-Phosphatidylcholine

### Metabolic Signatures of Exposure to Escitalopram/Citalopram—Four Weeks and Eight Weeks

Metabolites that reflected mitochondrial, neurotransmission and lipid metabolism changed significantly during the first four week of treatment (Table 3), including 4 amines (an increase asparagine and decreases in serotonin, sarcosine, spermine), 5 acylcarnitines (decreases in C5-OH(C3-DC-M), C8, C10, C12, C14:2), and 9 lipids (decreases in PC aa C38:6 and PC aa C40:6, and increases in PC aa C40:2, PC aa C42:2, PC ae C34:3, PC ae C36:3, lysoPC a C18:0 and lysoPC a C18:1).

TABLE 3
Statistically Significant 4-Week changes in metabolite
levels in Response to SSRI Treatment

| Metabolite | Estimated log2 FC | SE | p-value |
|---|---|---|---|
| Asparagine | 0.06 | 0.03 | 4.89E−02 |
| Sarcosine | −0.18 | 0.09 | 4.33E−02 |
| Serotonin | −1.45 | 0.16 | 1.22E−18 |
| Spermine | −0.10 | 0.05 | 4.56E−02 |
| C5—OH (C3-DC-M) | −0.26 | 0.06 | 3.07E−05 |
| C8 | −0.18 | 0.07 | 7.27E−03 |
| C10 | −0.17 | 0.07 | 9.37E−03 |
| C12 | −0.19 | 0.07 | 5.97E−03 |
| C14.2 | −0.14 | 0.07 | 3.62E−02 |
| lysoPC a C18:0 | 0.08 | 0.04 | 4.76E−02 |
| lysoPC a C18:1 | 0.07 | 0.04 | 4.96E−02 |
| PC aa C38:6 | −0.06 | 0.03 | 3.19E−02 |
| PC aa C40:2 | 0.07 | 0.03 | 1.35E−02 |
| PC aa C40:6 | −0.06 | 0.03 | 3.82E−02 |
| PC aa C42:2 | 0.06 | 0.03 | 3.14E−02 |
| PC ae C34:3 | 0.13 | 0.03 | 3.66E−05 |
| PC ae C36:3 | 0.06 | 0.03 | 3.55E−02 |
| SM C24:0 | 0.06 | 0.03 | 3.00E−02 |

Significance level was set to α = 0.05. The differences with p-values surpassing Bonferroni correction were bolded.

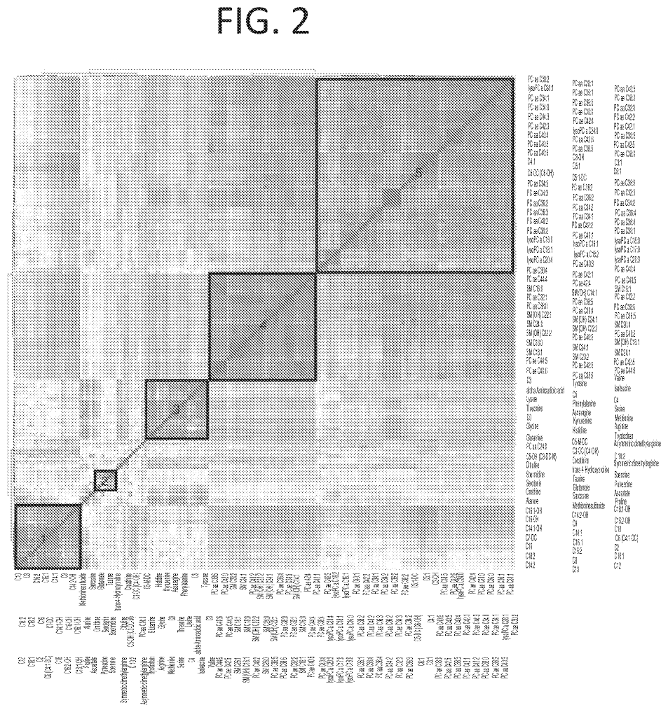

More pronounced metabolic changes were noted following the 8 weeks of SSRI treatment (FIGS. 2-4; Tables 4-5). Among the acylcarnitines, we observed an increased level of 3 short-chain acylcarnitines (C3, C4 and C5) and a decrease in medium and long-chain acylcarnitines (e.g., C8, C10, C12, C14:2, C16, C16:1, C18, C18:1, and C18:2). Within the classes of amino acids and biogenic amines, we observed a statistically significant increase in two amino acids (arginine and proline) and one biogenic amine (methionine sulfoxide), and a significant decrease in two biogenic amines (serotonin and sarcosine). Among the lipid classes, we observed a statistically significant up regulation of several phosphatidylcholines (PCs) (e.g., PC aa-C36:1, -C30:0, -C42:2; ether phosphatidylcholines, PC ae C34:3, -C38:2, -C36:3) and one sphingolipid (sphingomyelin [SM] C24:0). As depicted in FIG. 3, correlation analysis revealed that several metabolite changes in response to the drug exposure were highly correlated, especially notable amongst metabolites belonging to the same class. We observed five distinct clusters: cluster (1) mainly formed with medium- and long-chain acylcarnitines (e.g., C8, C9, C10, C12, C16); cluster (2) biogenic amines including serotonin, spermine, spermidine, taurine, putrescine, glutamate, omithine and sarcosine; cluster (3) short-chain acylcarnitines (C3, C4, C5), branched-chain amino acids (isoleucine and valine), kynurenine, arginine, glycine, tryptophan and glutamine; cluster (4) long-chain ether phospholipids; and cluster (5) a large number of phospholipids and lyso-phospholipids.

Concentrations of short-chain acylcarnitines decreased (C0, C3, C3-OH, C3:1 C4, C4:1, C5, C5-M-DC), with the exception of C2 which significantly increased. A decrease in many medium and long-chain acylcarnitines (C7:DC, C8, C10, C12, C14:1(OH), C14:2, C16, C16:1, C18, C18:1, C18:1.OH and C18:2) were also noted. Among the biogenic amines and amino acids, levels of arginine, citrulline, histidine, methionine, phenylalanine, trans-4-hydroxyproline increased and levels of aspartate, omithine, proline, tyrosine, kynurenine, methionine sulfoxide, sarcosine, serotonin and spermine decreased. Fifty metabolites from the classes of Glycerophospholipids and Sphingolipids significantly increased (e.g., PC as C24:0, PC aa C28:1, PC aa C30:0, PC ae C30:0, PC ae C34:0, SM C16:0, SM C24:0, SM C26:0) while only lysoPC a C16:0 and lysoPC a C20:4 significantly decreased.

TABLE 4
8-Week Changes in Metabolite Levels in Response to SSRI Treatment

| | Estimated | | | | Estimated | | |
|---|---|---|---|---|---|---|---|
| | Log2 | | | | Log2 | | |
| Metabolite | Difference | SE | p-value | Metabolite | Difference | SE | p-value |
| Arg | 0.52 | 0.08 | 4.20E-10 | lysoPC a | −0.13 | 0.03 | 2.41E-04 |
| | | | | C20:4 | | | |
| Orn | −0.26 | 0.06 | 4.80E-06 | lysoPC a | 0.12 | 0.03 | 6.01E-05 |
| | | | | C28:1 | | | |
| Met-SO | 0.38 | 0.08 | 6.07E-07 | PC aa | 0.11 | 0.03 | 1.01E-04 |
| | | | | C28:1 | | | |
| Sarcosine | −0.74 | 0.09 | 1.51E-15 | PC aa | 0.19 | 0.04 | 5.88E-06 |
| | | | | C30:0 | | | |
| Serotonin | −1.46 | 0.16 | 2.77E-18 | PC aa | 0.16 | 0.04 | 1.32E-05 |
| | | | | C34:3 | | | |
| C3 | 0.20 | 0.04 | 1.42E-06 | PC aa | 0.16 | 0.04 | 1.91E-04 |
| | | | | C34:4 | | | |
| C4 | 0.13 | 0.03 | 8.38E-05 | PC aa | 0.16 | 0.04 | 1.19E-05 |
| | | | | C36:1 | | | |
| C5 | 0.17 | 0.04 | 5.67E-05 | PC aa | 0.12 | 0.03 | 3.89E-05 |
| | | | | C40:2 | | | |
| C5—OH | −0.52 | 0.06 | 1.30E-15 | PC aa | 0.12 | 0.03 | 1.15E-05 |
| (C3—DC—M) | | | | C42:2 | | | |
| C8 | −0.31 | 0.07 | 1.15E-05 | PC ae | 0.13 | 0.03 | 1.44E-05 |
| | | | | C30:0 | | | |
| C10 | −0.33 | 0.07 | 1.38E-06 | PC ae | 0.13 | 0.03 | 2.76E-05 |
| | | | | C34:2 | | | |
| C12 | −0.31 | 0,07 | 2.73E-05 | PC ae | 0.18 | 0.03 | 1.38E-08 |
| | | | | C34:3 | | | |
| C14:2 | −0.35 | 0.07 | 7.17E-07 | PC ae | 0.10 | 0.03 | 1.03E-04 |
| | | | | C36:0 | | | |
| C16 | −0.22 | 0.04 | 2.96E-08 | PC ae | 0.13 | 0.03 | 4.91E-06 |
| | | | | C36:3 | | | |
| C16:1 | −0.32 | 0.05 | 3.54E-11 | PC ae | 0.15 | 0.03 | 3.85E-06 |
| | | | | C38:2 | | | |
| C18:1 | −0.52 | 0.06 | 9.49E-17 | SM C24:0 | 0.11 | 0.03 | 1.04E-04 |
| C18:2 | −0.55 | 0.06 | 1.63E-16 | | | | |

Signilicance level was set to a = 0.05/163 for Bonferroni correction.

TABLE 5
8-Week Changes in Metabolite Levels in Response to SSRI Treatment

| | | Estimated | | | | | Estimated | | |
|---|---|---|---|---|---|---|---|---|---|
| | | Log2 Change | | | | | Log2 Change | | |
| Class | Metabolite | (SE) | p-value | q-value | Class | Metabolite | (SE) | p-value | q-value |
| Acylcar- | C18:1 | −0.38(0.06) | 6.69E-09 | 4.99E-07 | PC aa | PC aa C36:1015(0.04) | | 4.26E-04 | 3.79E-03 |
| nitine | C16:1 | −0.31(0.05) | 9.18E-09 | 4.99E-07 | | PC aa C30:00.17(005) | | 4.41E-04 | 3.79E-03 |
| | C18:2 | −0.38(0.07) | 2.52E-08 | 1.03E-06 | | PC aa C42:2009(0.03) | | 7.55E-04 | 5.45E-03 |
| | C14:2 | −0.36(0.08) | 4.58E-06 | 1.22E-04 | | PC aa C34:40.16(005) | | 7.69E-04 | 5.45E-03 |
| | C16 | −0.21(0.05) | 5.26E-06 | 1.22E-04 | | PC aa C34:3013(0.04) | | 1.00E-03 | 6.13E-03 |
| | C10 | −0.33(0.08) | 2.92E-05 | 5.94E-04 | | PC aa C40:20.10(003) | | 1.01E-03 | 6.13E-03 |
| | C12 | −0.32(0.08) | 8.86E-05 | 1.55E-03 | | PC aa C38:6-0.08(0.03) | | 3.84E-03 | 1.84E-02 |
| | C8 | −0.31(0.08) | 1.10E-04 | 1.64E-03 | | PC aa C24:00.12(004) | | 4.92E-03 | 2.00E-02 |
| | C5—OH | −0.24(0.06) | 2.24E-04 | 3.04E-03 | | PC aa C28:1 0.08(0,03) | | 6.90E-03 | 2.62E-02 |
| | (C3—DC—M) | | | | | | | | |
| | C3 | 0.16(0.04) | 3.24E-04 | 3.61E-03 | | PC aa C36:6011(0.04) | | 9.60E-03 | 3.48E-02 |
| | C14:1-OH | −0.14(0.04) | 4.09E-04 | 3.79E-03 | | PC aa C42:40.07(003) | | 1.11E-02 | 3.92E-02 |
| | C7-DC | −0.17(0.05) | 5.08E-04 | 4.14E-03 | | PC aa C36:30.04(0.02) | | 1.56E-02 | 5.07E-02 |
| | C5 | 0.13(0.04) | 2.47E-03 | 1.34E-02 | | PC aa C32:10.13(006) | | 2.28E-02 | 7.01E-02 |
| | C2 | −0.16(0.06) | 3.46E-03 | 1.71E-02 | | PC aa C36:2003(0.02) | | 3.40E-02 | 9.54E-02 |
| | C16:1-OH | −0.13(0.05) | 4.66E-03 | 2.00E-02 | | PC aa C38:30.08(004) | | 3.97E-02 | 1.04E-01 |
| | C16:2-OH | −0.13(0.05) | 4.71E-03 | 2.00E-02 | | PC aa C36:5009(0.04) | | 4.70E-02 | 1.19E-01 |
| | C18 | −0.11(0.04) | 4.96E-03 | 2.00E-02 | PC ae | PC ae C34:3 | 0.17(0.03) | 1.29E-06 | 4.21E-05 |
| | C18:1-OH | −0.14(0.05) | 4.96E-03 | 2.00E-02 | | PC ae C38:2 | 0.13(0.04) | 3.46E-04 | 3.61E-03 |
| | C14:1 | −0.10(0.04) | 5.03E-03 | 2.00E-02 | | PC ae C36:3 | 0.12(0.03) | 3.54E-04 | 3.61E-03 |
| | C4 | 0.09(0.04) | 9.16E-03 | 3.39E-02 | | PC ae C34:2 | 0.11(0.03) | 8.87E-04 | 6.02E-03 |
| | C0 | 0.06(0,03) | 2.75E-02 | 8.00E-02 | | PC ae C30:0 | 0.11(0.03) | 1.02E-03 | 6.13E-03 |
| | C5—M—DC | 0.14(0.06) | 3.09E-02 | 8.84E-02 | | PC ae C42:2 | 0.08(0.03) | 1.13E-02 | 3.92E-02 |
| Amino | Arginine | 0.29(0.09) | 7.13E-04 | 5.45E-03 | | PC ae C36:2 | 0.07(0.03) | 1.65E-02 | 5.27E-02 |
| Acids | Proline | 0.13(0.04) | 3.41E-03 | 1.71E-02 | | PC ae C36:5 | 0.07(0.03) | 2.65E-02 | 7.84E-02 |
| | Tyrosine | 0.10(0.04) | 1.54E-02 | 5.07E-02 | | PC ae C36:0 | 0.06(0.03) | 3.52E-02 | 9.65E-02 |
| | Citrulline | 0.09(0.04) | 1.84E-02 | 5.76E-02 | | PC ae C32:2 | 0.05(0.03) | 4.74E-02 | 1.19E-01 |
| | Phenylalanine | 0.06(0.03) | 4.34E-02 | 1.12E-01 | Sphingolipids | SM C18:1 | −0.10(0.03) | 1.82E-03 | 1.03E-02 |
| | Serotonin | −1.77(0.17) | 1.55E-21 | 2.53E-19 | | SM C24:0 | 0.09(0.03) | 4.14E-03 | 1.93E-02 |
| | Sarcosine | −0.34(0.09) | 9.54E-05 | 1.55E-03 | | SM C18:0 | −0.06(0.03) | 3.55E-02 | 9.65E-02 |
| Biogenic | Methionine sulfoxi | 0.31(0.08) | 3.34E-04 | 3.61E-03 | | SM C20:2 | −0.08(0.04) | 3.68E-02 | 9.82E-02 |
| Amines | trans-4- | 0.15(0.07) | 2.50E-02 | 7.53E-02 | Lyso PCs | lysaPC a | −0.11(0.04) | 1.82E-03 | 1.03E-02 |
| | hydroxyproline | | | | | C20:4 | | | |
| | | | | | | lysoPC a | 0.09(0.03) | 2.90E-03 | 1.53E-02 |
| | | | | | | C28:1 | | | |
| | | | | | | lysoPC a | 0.08(0.03) | 5.79E-03 | 2.25E-02 |
| | | | | | | C24:0 | | | |
| | | | | | | lysoPC a | 0.08(0.03) | 1.48E-02 | 5.02E-02 |
| | | | | | | C28:0 | | | |

Significance level was set to a = 0.05. p-values obtained from linear mixed-effect models adjusted for age, sex, baseline HRSD₁₇ and
antidepressant. Metabolites surpassing Benjamini-Hochberg correction (q-value <0.05) are bolded. Abbreviations: SSRI: Selective Serotonin
Reuptake Inhibitor, HRSD₁₇: 17-item Hamilton Rating Scale for Depression, PC: Phosphatidylcholines, SM: Sphingomyelin, LysoPC: Lyso-
Phosphatidylcholines. Metabolite abbreviations are spelled out in Table 2

Association of Metabolite Levels with Depression Severity: Baseline, 8 Weeks and Change

We examined the association of change in HRSD₁₇ with log 2-fold change in metabolite levels from baseline to week 8, adjusting for the covariates of age and sex. The associations with uncorrected p-value <0.05 included: one acylcarnitine (C5-M-DC), four amines (histidine, proline, kynurenine and trans-4-hydroxyproline), seven PC aas and seven PC aes. Change in all of these metabolites were inversely associated with change in HRSD₁₇ (Table 6).

TABLE 6
Significant Associations Between Change in HRSD₁₇ Score and
Change in Metabolite Levels Over 8 Weeks of Treatment

| | | Effect | | | |
|---|---|---|---|---|---|
| Class | Metabolite | Size (β) | SE | p-value | q-value |
| Acylcarnitines | C5-M-DC | −0.203 | 0.087 | 2.09E−02 | 1.94E−01 |
| Amino Acids | Histidine | −0.188 | 0.086 | 3.09E−02 | 2.33E−01 |
| | Proline | −0.183 | 0.086 | 3.57E−02 | 2.33E−01 |
| Biogenic | Kynurenine | −0.287 | 0.084 | 8.11E−04 | 6.61E−02 |
| Amines | trans-4- | | | | |
| | hydroxyproline | −0.232 | 0.086 | 8.11E−03 | 1.56E−01 |
| PC aas | PC aa C36:3 | −0.245 | 0.085 | 4.52E−03 | 1.55E−01 |
| | PC aa C36:2 | −0.242 | 0.085 | 5.35E−03 | 1.55E−01 |
| | PC aa C34:2 | −0.236 | 0.085 | 6.43E−03 | 1.55E−01 |
| | PC aa C34:1 | −0.216 | 0.086 | 1.28E−02 | 1,67E−01 |
| | PC aa C36:4 | −0.215 | 0.086 | 1.33E−02 | 1.67E−01 |
| | PC aa C36:1 | −0.195 | 0.085 | 2.39E−02 | 2.05E−01 |
| | PC aa C40:2 | −0.186 | 0.086 | 3.21E−02 | 2.33E−01 |
| PC aes | PC ae C38:2 | −0.297 | 0.083 | 5.12E−04 | 6.61E−02 |
| | PC ae C36:3 | −0.244 | 0.085 | 4.52E−03 | 1.55E−01 |
| | PC ae C34:3 | −0.225 | 0.085 | 9.08E−03 | 1.56E−01 |
| | PC ae C36:2 | −0.210 | 0.085 | 1.52E−02 | 1.77E−01 |
| | PC ae C34:2 | −0.208 | 0.086 | 1.66E−02 | 1.80E−01 |
| | PC ae C38:3 | −0.202 | 0.085 | 1.96E−02 | 1.94E−01 |
| | PC ae C36:1 | −0.183 | 0.086 | 3.48E−02 | 2.33E−01 |
| Lyso PC | lysoPC a C18:1 | −0.233 | 0.085 | 6.68E−03 | 1.55E−01 |
| | lysoPC a C18:0 | −0.222 | 0.085 | 9.56E−03 | 1.56E−01 |
| | lysoPC a C17:0 | −0.213 | 0.085 | 1.33E−02 | 1.67E−01 |
| | lysoPC a C18:2 | −0.200 | 0.086 | 2.14E−02 | 1.94E−01 |
| | lysoPC a C20:3 | −0.191 | 0.086 | 2.71E−02 | 2.21E−01 |
| | lysoPC a C16:0 | −0.183 | 0.085 | 3.43E−02 | 2.33E−01 |

P-values were obtained from linear regression models adjusted for age and sex. Significance level was set to 0 = 0.05. Abbreviations: HR5D₁₇: 17-item Hamilton Rating Scale for Depression, PC: Phosphatidylcholine, LysoPC: Lyso-Phosphatidylcholine.

At baseline (n=240), higher levels of arginine, asparagine and threonine and lower levels of PC ae C36:3, PC ae C36:4 and PC aa C38:3 were associated with higher HRSD₁₇ score after adjusting for age and sex. At week 8 (n=144), HRSD₁₇ scale was negatively associated with isoleucine, phenylalanine, sarcosine, serotonin and PC aa C40:6 (Table 7). In addition, we examined association of change in HRSD₁₇ with log 2 fold-change in metabolites (N=127), which yielded significant associations for 12 metabolites. Kynurenine, t4.OH.Pro, lysoPC a C18:1, PC as C34:1, PC aa C34:2, PC as C36:2, PC aa C36:3, PC aa C36:4, PC ae C34:3, PC ae C36:2, PC ae C36:3, PC ae C38:2 were negatively associated with change in HRSD₁₇. No metabolites were positively associated significantly with change in HRSD₁₇ (Table 8).

TABLE 7
Metabolites significantly different between Remitters and Non-responders

| Baseline | | | 4 weeks | | | 8 weeks | | |
|---|---|---|---|---|---|---|---|---|
| | Estimated | | | Estimated | | | Estimated | |
| | Log2 | | | Log2 | | | Log2 | |
| | Difference | p- | | Difference | p- | | Difference | p- |
| Metabolite | (SE) | value | Metabolite | (SE) | value | Metabolite | (SE) | value |
| Serotonin | 0.81 (0.35) | 2.21E- | Arginine | −0.60 | 1.29E-02 | Putrescine | 0.49 (0.22) | 2.33E-02 |
| | | 02 | | (0.24) | | | | |
| C3 | 0.23 (0.10) | 2.35E- | Sarcosine | 1.03 (0.27) | 1.89E-04 | C8 | 0.55 (0.21) | 9.28E-03 |
| | | 02 | | | | | | |
| | | | C5(OH)—- | 0.46 (0.19) | 1.56E-02 | C9 | 0.21 (0.11) | 4.92E-02 |
| | | | C3—-DC—-M | | | | | |
| | | | C18:1 | 0.36(0.18) | 4.55E-02 | C12 | 0.47 (0.22) | 3.46E-02 |
| | | | C18:2 | 0.57(0.19) | 3.71E-03 | lysoPC a | 0.42 (0,15) | 6.04E-03 |
| | | | | | | C18:2 | | |
| | | | | | | PC aa | 0.09 (0.04) | 2.87E-02 |
| | | | | | | C34:2 | | |
| | | | | | | PC aa | 0.09 (0.04) | 3.08E-02 |
| | | | | | | C36.2 | | |
| | | | | | | PC aa | 0.21 (0.09) | 2.14E-02 |
| | | | | | | C42.2 | | |
| | | | | | | PC ae | 0.24 (0.12) | 4.57E-02 |
| | | | | | | C34.3 | | |
| | | | | | | PC ae | 0.21 (0.09) | 2.59E-02 |
| | | | | | | C36.2 | | |
| | | | | | | PC ae | 0.20 (0.09) | 3.71E-02 |
| | | | | | | C42.3 | | |
| | | | | | | SM (OH) | 0.21 (0.10) | 2.87E-02 |
| | | | | | | C14:1 | | |
| | | | | | | SM (OH) | 0.25 (0.11) | 2.10E-02 |
| | | | | | | C22:1 | | |
| | | | | | | SM (OH) | 0.23 (0.10) | 3.00E-02 |
| | | | | | | C24:1 | | |
| | | | | | | SM 024:0 | 0.22 (0.11) | 4.20E-02 |

Linear mixed effect models were used to assess the significance of visit by treatment outcome
interaction. Models were adjusted for age, sex and baseline HRSD₁₇ score. Significance level were set
to a = 0.05.

TABLE 8
Association between Change in Metabolite Levels and Change in
HRSD₁₇ Scores Over 8 Weeks of Treatment

| Metabolite | Effect Size (β) | SE | p-value |
|---|---|---|---|
| Kyrurenine | −0.306 | 0.086 | 5.43E−04 |
| t4(OH) Pro | −0.231 | 0.089 | 1.07E−02 |
| C5-M-DC | −0.182 | 0.090 | 4.57E−02 |
| lysoPC a 018:1 | −0.183 | 0.089 | 4.18E−02 |
| PC aa C34:1 | −0.181 | 0.089 | 4.41E−02 |
| PC aa C34:2 | −0.195 | 0.089 | 3.08E−02 |
| PC aa C36:2 | −0.196 | 0.089 | 2.96E−02 |
| PC aa 036:3 | −0.194 | 0.089 | 3.12E−02 |
| PC aa C36:4 | −0.180 | 0.089 | 4.57E−02 |
| PC ae 034:3 | −0.181 | 0.089 | 4.36E−02 |
| PC ae 036:2 | −0.177 | 0.089 | 4.87E−02 |
| PC ae C36:3 | −0.203 | 0.089 | 2.36E−02 |
| PC ae C38:2 | −0.256 | 0.087 | 3.89E−03 |

Linear regression models adjusted for age and sex were used to assess associations. Significance level was set to α = 0.05.

Metabolic Signatures of Remission Vs. Non-Response at Baseline, 4 Weeks and 8 Weeks

At baseline, levels of serotonin and C3 were significantly higher in the remitters (p<0.05). At 4 weeks, arginine levels were lower in the remitters while levels of sarcosine, C5(OH)-C3-DC-M, C18:1 and C18:2 were higher. At week 8, levels of 3 long-chain acyl-carnitines (C8, C9 and C12), 1 biogenic amine (Putrescine), 11 Sphingolipids and Glycerophospholipids (lysoPC a C18:2, PC aa C34:2, PC aa C36.2, PC aa C42.2, PC ae C34.3, PC ae C36.2, PC ae C42.3, SM (OH) C14:1, SM (OH) C22:1, SM (OH) C24:1, SM C24:0) were all significantly higher in the remitters.

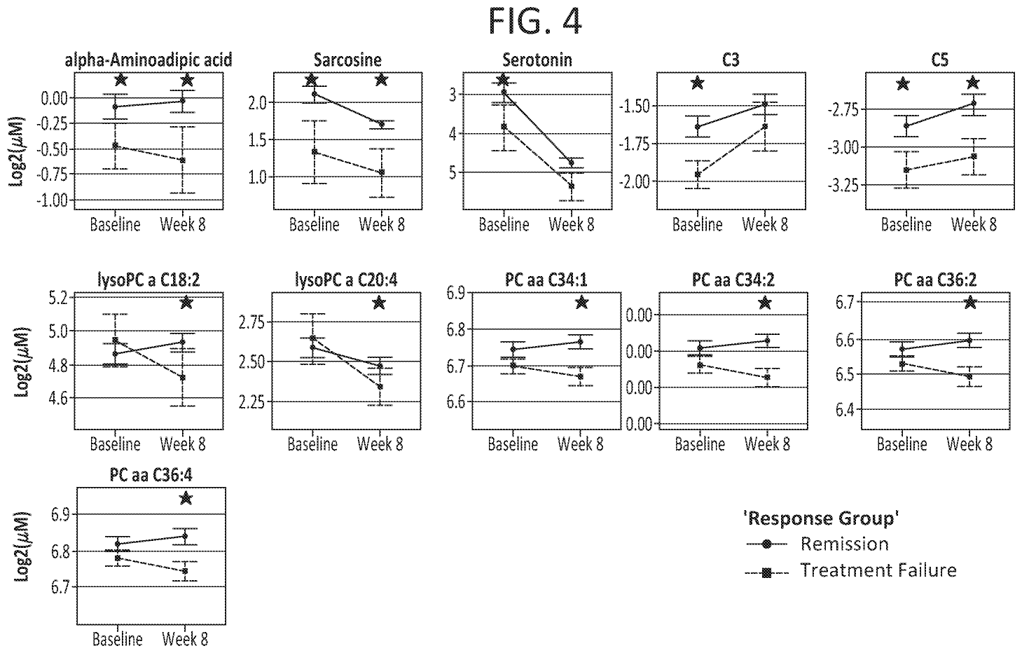

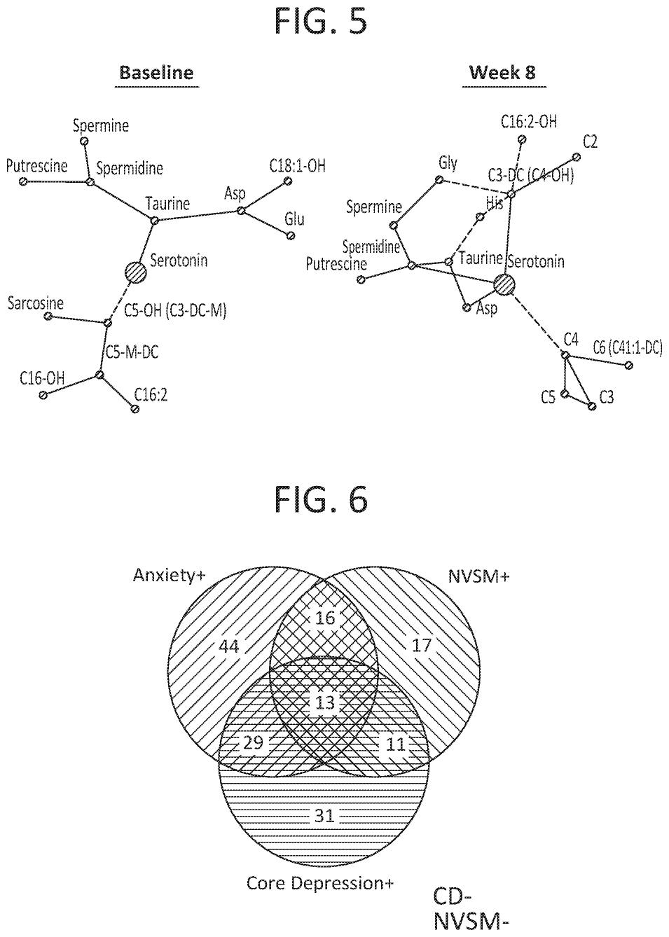

We compared the remitter (n=64) and treatment failure (n=12) groups at baseline and after 8 weeks of SSRI treatment. Eleven metabolites were significantly different (unadjusted p-value <0.05) between the two groups at either baseline or week 8 (FIG. 4; Table 7). Levels of two short-chain acylcarnitines (C3 and C5) and two biogenic amines (alpha-amino adipic acid and sarcosine) were higher at baseline in the remitters and remained higher at the end of treatment. Serotonin and C3 were significantly different at baseline, but not at week 8. SSRI treatment resulted in differential regulation of four phosphatidylcholines (PC aa C34:2, PC aa C36:2, PC aa C36:4 and PC aa C43:1) and two lysoPCs (lysoPC a C18:2 and lysoPC a C20:4) between the two groups at week 8.

We addressed 4 important theoretical and clinical questions by using metabolomics that reflect mitochondrial function, neurotransmission and lipid metabolism—all of which have been implicated in MDD or the effects of Citalopram/Escitalopram antidepressant medication. We found that exposure over 8 weeks an acute treatment of SSRI had significant effects by increasing in short-chain acylcarnitines, Arginine, Citrulline, Histidine, Methionine, Phenylalanine, trans-4-hydroxyproline, and fifty metabolites from the classes of Glycerophospholipids and Sphingolipid, and decreasing in one short-chain (C2) and many medium and long-chain acylcarnitines, and in levels of Aspartate, Omithine, Proline, Tyrosine, Kynurenine, Methionine sulfoxide, Sarcosine, Serotonin and Spermine after 8 weeks of SSRIs treatment. At baseline, of these metabolite domains, Arginine, Asparagine and Threonine was positively associated HRSD₁₇ score, while some Glycerophospholipids was negatively associated. At 8 weeks, depressive symptoms HRSD₁₇ scale was negatively associated with Isoleucine, Phenylalanine, Sarcosine, Serotonin and Kynurenine, t4.OH.Pro, and many Glycerophospholipids, which suggesting the relevance of metabolites to the therapeutic effects of the drugs. At baseline, levels of Serotonin and C3 were significantly higher in the remitters compare to non-responders. At the end of therapy, levels of 3 long-chain acyl-carnitine (C8, C9 and C12), 1 biogenic amine (Putrescine), 11 Sphingolipids and Glycerophospholipids were significantly higher in the remitters compared to complete non-responders.

### Acylcarnitines

In our study, after 8 weeks of SSRI treatment, the short-chain acylcarnitines, specifically propionyl carnitine (C3), butyryl/isobutyryl carnitine (C4) and isovaleryl/methylbutyryl carnitine (C5)—were significantly increased while acetyl carnitine (C2) levels were decreased. In a rat model of depression, incomplete β-oxidation of fatty acids has been associated with elevated medium- and long-chain acylcarnitines (Chen et al. J. Pharmac. Biomed. Anal. 89, 122-129 (2014). This finding is in line with our observation of decreased medium- and long-chain acylcarnitines after 8 weeks of anti-depressant therapy and suggests that the drug may act to restore the mitochondrial β-oxidation process with greater utilization of the medium- and long-chain acylcarnitines. Remarkably, in our study, the plasma ratio of long-chain acylcarnitines to free carnitine (C16:0+C18:0/C0) was significantly reduced at week 8 (p-value <3.4E-06). These findings suggest a similar pattern of mitochondrial dysfunction and a drug-induced functional restoration thereof across the spectrum of neuropsychiatric disorders.

We also found that the changes in short-chain acylcarnitines (C3, C4, C5) over 8 weeks of SSRI treatment were correlated with changes in branched-chain amino acids (BCAAs; isoleucine and valine). In our study, we also observed that BCAA levels increased with the antidepressant exposure, though not significantly, and that remitters had higher baseline BCAA levels that further increased post-treatment compared to the treatment failure group.

### Amino Acids and Biogenic Amines

After 8 weeks of SSRI treatment, we noted significant perturbations in the urea cycle and nitric oxide cycle metabolites. In our study, after 8 weeks of drug exposure, plasma arginine levels increased significantly, while omithine levels showed a trend to be lower compared to baseline. The ratios of citrulline/arginine (p-value <0.013) and asymmetric dimethyl arginine/arginine (ADMA/Arg, p-value <8.62E-05) were significantly lower compared to baseline, both of which may indicate potential increases in activity of Nitric Oxide Synthase and increased production of Nitric Oxide (NO) post-treatment

The biogenic amine serotonin decreased significantly with the drug exposure. We found that after 4 weeks of SSRI exposure, the decrease in sarcosine levels was correlated with the decrease in serotonin levels. Sarcosine and serotonin were also highly correlated to each other in our study.

Additionally, at baseline, their levels were significantly higher in the remitters compared to the treatment failure group and remained higher even at the end of treatment.

Among the other members of this class, changes in histidine and kynurenine levels from baseline to week 8 were inversely associated with changes in depressive symptoms scores (HRSD₁₇). We also found significant increases in methionine sulfoxide (MetSO; FIG. 3) and the ratio of methione sulfoxide/methionine (MetSO/Met; p-value <0.0008) from baseline to week 8.

### Lipids

Among the lipids studied, the perturbations amongst the phosphatidylcholines (PCs) containing either the diacyl or the alkyl-acyl moieties were strong, and many of them were inversely associated with changes in the depressive symptom scores from baseline to week 8 (FIG. 3, Table 6). Furthermore, upon stratification between remitters and participants with treatment failure, several interesting trends emerged. First, the lysophosphatidylcholines containing a single fatty acyl chain (lysoPCs-C16:0, -C17:0, -C18:0, -C18:1, C18:2, C20:3 and C20:4) mostly showed downward trajectories from baseline to week 8 among the treatment failure group but not so among the remitters. Second, several diacyl PCs that showed inverse correlations to changes in depression scores (FIG. 3 and Table 6) (e.g., PC aa-C34:1, 34:2, 36:2, 36:3, 36:4, 38:4) showed very similar trajectories in that the remitters all had higher baseline levels that increased further at week 8, compared to the treatment failure group which showed very similar downward trajectories (FIG. 3). Lastly, the ether-phosphatidylcholines as a group showed a distinct pattern of perturbation: the treatment failure group mostly had significantly higher baseline levels compared to the remitters, and these metabolites then followed downward trajectories over the treatment period whereas the remitters either showed an increase or stayed unchanged. Overall, the lipids displayed remarkable differences between the remitters and treatment failure group regarding how the two groups responded to the drug. While it is difficult to come up with plausible explanations for such observed differences, there seems to be an important role for these lipids in contributing to drug response.

Of special interest is the distinct pattern we observed among the ether phospholipids. The distinctive chemical feature of the ether lipids is the ether bond at the sn-1 position of the glycerol backbone where a fatty alcohol is attached as opposed to the more common diacyl moiety containing phospholipids. The initial stages of ether-lipid synthesis occur in the peroxisomes, including the rate-limiting enzymatic processes. It is also possible that peroxisomal disorders and subsequent metabolic resilience result in the distinct patterns observed in the trajectories of ether-phospholipids in remitters vs. those in the treatment failure group depressed states and provide a way of characterizing the effects of antidepressants on metabolic pathways. This approach may ultimately inform therapeutic choices, thus reducing trial-and-error prescribing and contributing to personalizing therapeutic treatment for patients with MDD.

### Example 2

### Study Design and Participants

A sample 803 MDD patients from the Mayo Clinic NIH-Pharmacogenomics Research Network-Antidepressant Pharmacogenomics Medication Study (PGRN-AMPS) was used. Patient selection, symptomatic evaluation, and blood sample collection for the PGRN-AMPS clinical trial have been described in our previous work. MDD symptoms were assessed with HRSD₁₇ at baseline, and 8-weeks on SSRI treatment. Blood samples were collected at these same time periods. This study data extraction protocol has followed the STORBE guidelines.

### Identifying Subtypes

To address this heterogeneity, we first identified clinical phenotypes based on Research Domain Criteria (RDoC) conceptual models and by reaching expert clinician consensus (MAF, AJR, BWD). We have utilized data of well-defined MDD patients treated with citalopram or escitalopram—a highly selective serotonin reuptake inhibitors (SSRIs) by using the HRSD₁₇ score as a primary outcome measure from a large clinical trial: The Mayo Pharmacogenomic Research Network Antidepressant Medication Pharmacogenomic Study (PGRN-AMPS) study. Initially, we used descriptive baseline features to define this phenotype. Then, we reported three phenotypes of depression that could represent RDoC domains and contracts: CD, NVSM and ANX. CD subtype represents RDoC domain of negative valence systems and construct of loss. We defined it based on the presence of symptoms severity of items 1 (depressed mood) and 7 (anhedonia) on HRSD₁₇. NVSM subtype represents RDoC domain of Arousal and Regulatory Systems and construct of sleep-wakefulness. We defined it based on the presence of symptoms severity of items 6 (late insomnia) and 12 (somatic gastrointestinal) on HRSD₁₇. ANX subtype represents Rdo domain of negative valence systems and potential threat (“Anxiety”). We defined it based on the presence of symptoms severity of items 9 (agitation), 10 (anxiety psychological), 11 (anxiety somatic) and 15 (hypochondriasis) on HRSD₁₇ (Table 9, FIG. 6). Subsequently, we attempted to identify whether the baseline metabolic profile (i.e., “metabotype”) differs among patients with the phenotype of MDD, and how these individuals will respond to treatment.

TABLE 9
Phenotype Symptom Constructs and Criteria Thresholds

| RDoC Construct | Phenotype (Mayo and Emory) | | HRSD-17 items |
|---|---|---|---|
| Loss | Core Depression (CD) | Symptoms | depressed mood #1 work and |
| | | | activities #7 |
| | | Criteria definition | score of 3 or 4 on both items #1 |
| | | | and #7 |
| Potential Threat | Anxiety (ANX)* | Symptoms | agitation #9; anxiety psychological |
| (“Anxiety”) | | | #10; anxiety somatic #11; |
| | | | hypochondriasis #15 |
| | | Criteria definition | total score ≥6 from items #9, #10, |
| | | | #11, and #15 |
| Sleep- | Neurovegetative | Symptoms | late insomnia #6; somatic |
| Wakefulness | Symptoms of | | gastrointestinal #12 |
| | Melancholia (NVSM) | Criteria definition | score of 1 or 2 on both items #6 |
| | | | and #12 |

### Metabolomic Profiling

Metabolites were measured with a targeted metabolomics approach using the AbsoluteIDQ p180 Kit (BIOCRATES Life Science AG, Innsbruck, Austria), with a ultra-performance liquid chromatography (UPLC)/MS/MS system (Acquity UPLC (Waters), TQ-S triple quadrupole MS/MS (Waters)) which provides measurements of up to 186 endogenous metabolites quantitatively (amino acids and biogenic amines) and semi-quantitatively (acylcarnitines, sphingomyelins, phosphatidylcholines and lysophosphatidylcholines across multiple classes). In this report we only focused on acylcarnitines. The AbsoluteIDQ® p180 kit has been fully validated according to European Medicine Agency Guidelines on bioanalytical method validation. Additionally, plates include an automated technical validation to approve the validity of the run and provide verification of the actual performance of the applied quantitative procedure including instrumental analysis. The technical validation of each analyzed kit plate was performed using MetIDQ® software based on results obtained and defined acceptance criteria for blank, zero samples, calibration standards and curves, low/medium/high-level QC samples and measured signal intensity of internal standards over the plate. This is a highly useful platform that was used in hundreds of publications, including several studies in MDD 36 37. De-identified samples were analyzed following the manufacturer's protocol, with metabolomics labs blinded to diagnosis and clinical data. List of acylcarnitines metabolites analyzed in the study is shown in Table 10.

TABLE 10
List of Acylcarnitines included in the analysis

| | |
|---|---|
| C0 | L-Carnitine |
| C2 | Acetyl-L-carnitine |
| C3 | Propionyl-L-carnitine |
| C3:1 | Propenyl-L-carnitine |
| C3-DC/C4-OH | Malonyl-L-carnitine/Hydroxybutyryl-L-carnitine |
| C3-DC-M/C5-OH | Methylmalonyl-L-carnitine/Hydroxyvaleryl-L- |
| | carnitine |
| C3-OH | Hydroxypropionyl-L-carnitine |
| C4 | Butyryl-L-carnitine |
| C4:1 | Butenyl-L-carnitine |
| C4:1-DC/C6 | Fumaryl-L-carnitine/Hexanoyl-L-carnitine |
| C5 | Valeryl-L-carnitine |
| C5:1 | Tiglyl-L-carnitine |
| C5:1-DC | Giutaconyl-L-carnitine |
| C5-DC/C6-OH | Glutaryl-L-carnitine/Hydroxyhexanoyl-L-carnitine |
| C5-M-DC | Methylglutaryl-L-carnitine |
| C6:1 | Hexeroyl-L-carnitine |
| C7-DC | Pimelyl-L-carnitine |
| C8 | Octanoyl-L-carnitine |
| C8:1 | Octenoyl-L-carnitine |
| C9 | Nonayl-L-carnitine |
| C10 | Decanoyl-L-carnitine |
| C10:1 | Decenoyl-L-carnitine |
| C10:2 | Decadienyl-L-carnitine |
| C12 | Dodecanoyl-L-carnitine |
| C12:1 | Dodecenoyl-L-carnitine |
| C12-DC | Dodecanedioyl-L-carnitine |
| C14 | Tetradecanoyl-L-carnitine |
| C14:1 | Tetradecenoyl-L-carnitine |
| C14:1-OH | Hydroxytetradecenoyl-L-carnitine |
| C14:2 | Tetradecadienyl-L-carnitine |
| C14:2-OH | Hydroxytetradecadienyl-L-carnitine |
| C16 | Hexadecanoyl-L-carnitine |
| C16:1 | Hexadecenoyl-L-carnitine |
| C16:1-OH | Hydroxyhexadecenoyl-L-carnitine |
| C16:2 | Hexadecadienyl-L-carnitine |
| C16:2-OH | Hydroxyhexadecadienyl-L-carnitine |
| C16-OH | Hydroxyhexadecanoyl-L-carnitine |
| C18 | Octadecanoyl-L-carnitine |
| C18:1 | Octadecenoyl-L-carnitine |
| C18:1-OH | Hydroxyoctadecenoyl-L-carnitine |
| C18:2 | Octadecadienyl-L-carnitine |

### Data Analyses

Preprocessing: All metabolite data were first checked for missing values (<limit of detection) and metabolites with >40% missing values were excluded from subsequent analysis. Each assay plate included a set of duplicates obtained by combining approximately 10 μL from the first 76 samples in the study (QC pool duplicates) to allow appropriate inter-plate abundance scaling based specifically on this cohort of samples. To adjust for the batch effects, a correction factor for each metabolite in a specific plate was obtained by dividing metabolite's SPQC global average by SPQC average within the plate. <LOD values were imputed using each metabolite's LOD/2 value followed by log 2 transformation. We checked for the presence of multivariate outlier samples by evaluating the squared Mahalanobis distance of samples in each platform. Samples with Mahalanobis distances exceeded the upper 0.05/n (with n: number of samples to adjust for multiple comparisons by Bonferroni correction) critical value of the Chi-squared distribution with m degrees of freedom where m is the number of metabolites in each platform were flagged as outlier. An additional 15 samples were removed after they were determined to be multivariate outliers. This resulted in an analysis data set containing 269 subjects, 537 samples and 163 metabolites.

### Statistical Analysis

Differences of demographic, and depression scores across the phenotypes were evaluated using F-test (for continuous variables) and Pearson Chi-squared test (for categorical variables). All analyses were performed in a metabolite-wise manner. All models included age, sex, enantiomer (Escitalopram/Citalopram) and HRSD-17. Presence of each phenotype (CD, NVSM and ANX) for each patient at baseline was stored at a binary variable. Baseline metabolite concentrations and changes in metabolite concentrations after SSRI treatment were tested. To examine the significance of differential abundance of metabolite levels across phenotypes at baseline and at the end of therapy, we fitted linear mixed effect models (random intercept) with log 2 metabolite levels as the dependent variable and interaction of phenotypes (8 level categorical variable: all possible combinations of [CD+|CD−][NVSM+|NVSM−][ANX+|ANX−]) and visit (3 level categorical variable: baseline; week 4; week 8) as independent variable while correcting for age, sex, HRSD₁₇ at each visit and enantiomer name (Escitalopram/Citalopram). Then, we used “emmeans” R package to compute the least squared means of the contrasts of interest at baseline and at 8 weeks (i.e., 1: CD+vs. phenotype (−); 2: NVSM+vs. phenotype (−); 3:ANX+vs. phenotype (−); 4:ANX+vs. CD+; 5: ANX+vs. NVSM+; 6: CD+vs. NVSM+) and their corresponding p-values. To examine the significance of 8 weeks metabolite abundance log 2-fold change in all subjects irrespective of phenotype, linear mixed effect model (random intercept) were fitted with log 2 metabolite levels as the dependent variable and visit (3 level categorical variable: baseline; week 4; week 8) as independent variable while correcting corrected for age, sex, baseline HRSD₁₇ and enantiomer name (Escitalopram/Citalopram), followed by testing the contrast of interest (week 8 vs. baseline) using the emmeans function.

### Patient Characteristics

Plasma metabolite data were available from 240 MDD patients. Demographic and clinical characteristics are detailed in Table 11.

TABLE 11
Baseline demographics

| | | | | | Baseline | |
|---|---|---|---|---|---|---|
| | AGE | | Gender Male | | HRSD-17 | |
| Phenotype | Mean (SD) | | % (N) | | Mean (SD) | |
| All phenotype (N = 171) | 39.77 | (13.63) | 33% | (56) | 20.30 | (3.68) |
| CD+ (N = 31) | 43.10 | (13.97) | 32% | (10) | 21.48 | (3.88) |
| NVSM+ (N = 17) | 36.55 | (14.77) | 12% | (2) | 21.53 | (3.08) |
| ANX+ (N = 44) | 36.81 | (12.34) | 34% | (15) | 22.25 | (3.26) |
| No Phenotype (N = 79) | 40.80 | (13.74) | 37% | (29) | 18.48 | (3.08) |

HRSD₁₇: The Hamilton Depression Rating Scale - 17 items.

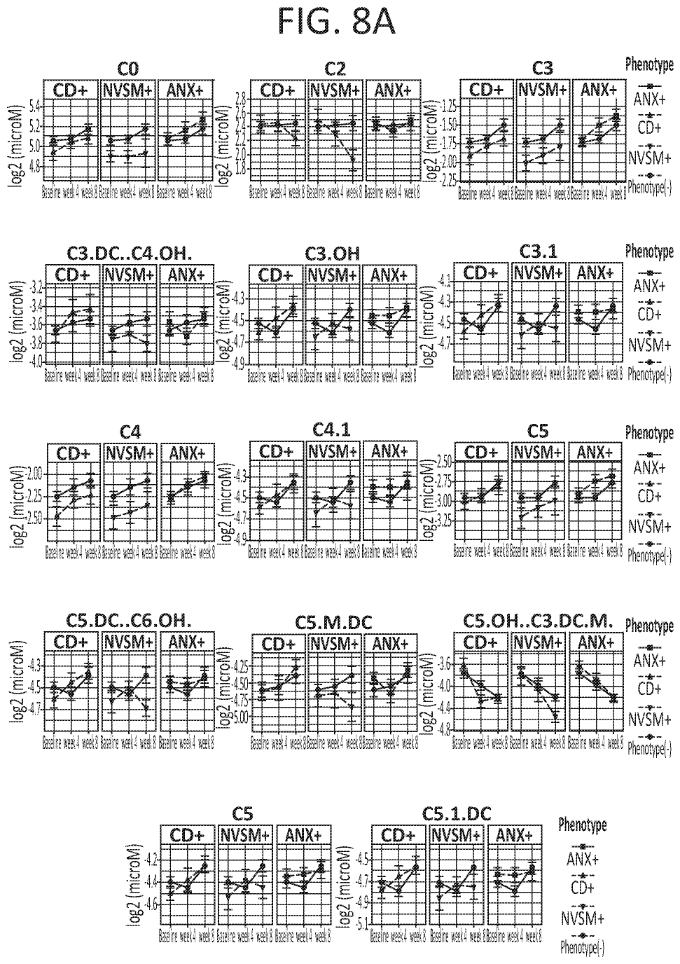

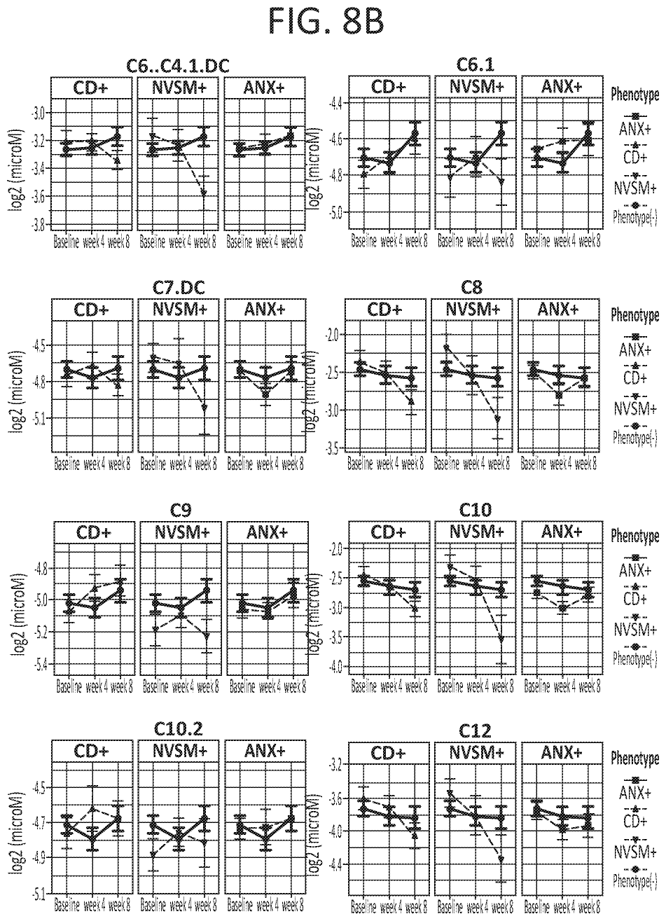

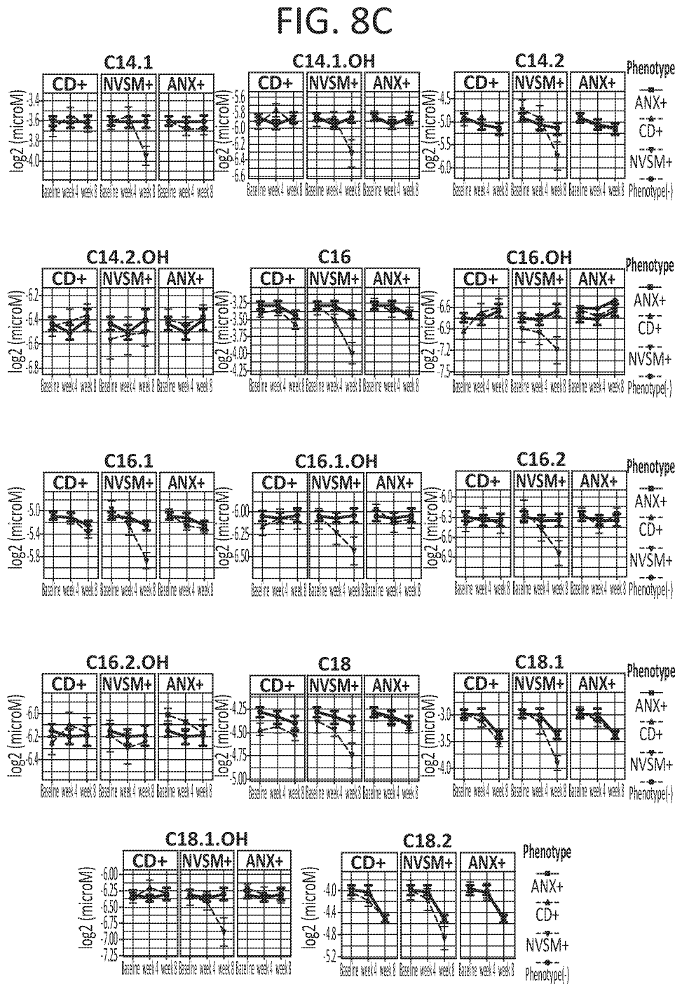

Acylcarnitines concentrations at baseline distinguish Phenotype (+) from Phenotype (−) at Baseline Phenotypes (+) compare to phenotype (−) patients show some unique short-, medium- and long-chain acylcarnitines signatures that separates between these phenotypes. CD+ patients demonstrate significantly lower in the levels of the short-chain C4 and long-chain C18 of acylcarnitines compare to phenotype (−) patients. ANX+ patients demonstrate significantly lower in the levels of the short-chain C7.DC and long-chain C16.2 of acylcarnitines compare to phenotype (−) patients (Table 12, FIGS. 8A-C).
Acylcarnitines Concentrations at Baseline Distinguish Phenotype (+) from Phenotype (+) at Baseline

While all phenotypes (+) showed similar trends of acylcarnitines at baseline, CD+ patients demonstrate significantly lower in the levels of short-chain C0, C3, C3-OH, C3:1, C4:1, C5:1, C5-DC-C6-OH, C5:1, long-chain C16-OH, C16:1-OH, C16:2-OH and C18 acylcarnitines compare to ANX+. Moreover, in NVSM+ compare to ANX+, short-chain C3-OH, C3:1, C4:1, C5-DC-C6-OH and long-chain C16-OH acylcarnitines levels were significantly lower, and C10 significantly higher. Significant p-values ranged from <0.05 to 0.01 (Table 12, FIG. 8A-C).

TABLE 12
Significantly Different Metabolite Levels Between Phenotype (+) and (−), and Phenotype (+) and (+) at Baseline and 8 weeks.

| | Baseline | | | | | | 8 weeks | | |
|---|---|---|---|---|---|---|---|---|---|
| | CD+* vs Phenotype (−) | | | ANX+* vs Phenotype (−) | | | NVSM+* vs Phenotype (−) | | |
| | Estimated | | | Estimated | | | Estimated | | |
| Acylcarnitine | Log2 | | | Log2 | | | Log2 | | |
| Metabolites | Difference | SE | p-value | Difference | SE | p-value | Difference | SE | p-value |
| C0 | | | | | | | | | |
| C2 | | | | | | | 0.43 | 0.19 | 0.02 |
| C3 | | | | | | | | | |
| C3.OH | | | | | | | | | |
| C3.1 | | | | | | | | | |
| C4 | 0.23 | 0.10 | 0.02 | | | | | | |
| C4.1 | | | | | | | | | |
| C5.M.DC | | | | | | | 045 | 0.23 | 0.047 |
| C5.DC.C6.OH. | | | | | | | 0.25 | 0.12 | 0.04 |
| C5.1 | | | | | | | | | |
| C5.1.DC | | | | | | | | | |
| C6.C4.1.DC. | | | | | | | 0.30 | 0.12 | 0.01 |
| C7:DC | | | | 0.32 | 0.14 | 0.03 | 0.36 | 0.18 | 0.04 |
| C10 | | | | | | | 0.73 | 0.25 | 0.00 |
| C14.1 | | | | | | | 0.31 | 0.13 | 0.01 |
| C14.1.OH | | | | | | | 0.39 | 0.13 | 0.00 |
| C14.2 | | | | | | | 0.53 | 0.25 | 0.04 |
| C16 | | | | | | | 0.46 | 0.15 | 0.00 |
| C16.OH | | | | | | | 0.51 | 0.18 | 0.01 |
| C16.1 | | | | | | | 0.51 | 0.17 | 0.00 |
| C16.1.OH | | | | | | | 0.33 | 0.16 | 0.04 |
| C16.2 | | | | 0.34 | 0.16 | 0.03 | 0.41 | 0.20 | 0.04 |
| C16.2.OH | | | | | | | | | |
| C18 | 0.17 | 0.09 | 0.047 | | | | | | |
| C18.1.OH | | | | | | | 0.52 | 0.16 | 0.00 |

CD: Core depression, ANX: Anxiety, NVSM: Neurovegetative Symptom of Melancholia; Linear mixed effect models were used to assess the
significance of visit by treatment outcome interaction. Models were adjusted for age, sex, treatment and baseline HRSD₁₇ score. Significance
level were set to a = 0.05 not corrected for Bonferroni corrections.

TABLE 10
Significantly Different Metabolite Levels Between Phenotype (+) and (−), and Phenotype (+) and (+) at baseline and 8 weeks

| | Baseline | | | | | | | | | 8 weeks | | | | | |
|---|---|---|---|---|---|---|---|---|---|---|---|---|---|---|---|
| | CD+* vs ANX+* | | | NVSM+* vs ANX+* | | | CD+* vs NVSM+* | | | CD+* vs ANX+* | | | NVSM+* vs ANX+* | | |
| | Esti- | | | Esti- | | | Esti- | | | Esti- | | | Esti- | | |
| | mated | | | mated | | | mated | | | mated | | | mated | | |
| | Log2 | | | Log2 | | | Log2 | | | Log2 | | | d Log2 | | |
| Acylcarrntine | Differ- | | p- | Differ- | | p- | Differ- | | p- | Differ- | | p- | Differ- | | p- |
| Metabolites | ence | SE | value | ence | SE | value | ence | SE | value | ence | SE | value | ence | SE | value |
| C0 | −0.16 | 0.08 | 003 | | | | | | | | | | −0.24 | 0.01 | 0.04 |
| C2 | | | | | | | | | | −0.31 | 0.15 | 0.04 | −0.54 | 0.20 | 0.01 |
| C3 | −0.26 | 0.11 | 0.02 | | | | | | | −0.27 | 0.13 | 0 04 | −0.36 | 0.17 | 0 03 |
| C3 | −0.26 | 0.11 | 0.02 | | | | | | | | | | | | |
| C3.OH | −0.19 | 0.09 | 0.03 | −0.22 | 0.11 | 0.04 | | | | | | | | | |
| C3.1 | −0.23 | 0.11 | 0.04 | −0.25 | 0.11 | 0.03 | | | | | | | | | |
| C4.1 | −0.20 | 0.08 | 0.02 | −0.26 | 0.12 | 0.03 | | | | | | | | | |
| C5.DC..C6.OH | −0.16 | 0.08 | 0.03 | −0.20 | 0.10 | 0.047 | 0.32 | 0.13 | 0.02 | | | | −0.31 | 0.13 | 0.02 |
| C5.1 | −0.17 | 0.09 | 0.04 | | | | | | | | | | | | |
| C5.1.DC | | | | −0.24 | 0.11 | 0.03 | | | | | | | | | |
| C5.M.DC | | | | | | | 0.60 | 0.25 | 0.02 | | | | −0.66 | 0.24 | 0.01 |
| C6..C4.1.DC. | | | | | | | | | | −0.22 | 0.10 | 0 03 | −0.38 | 0.13 | 0.00 |
| C7.DC | | | | | | | | | | | | | −0.37 | 0.18 | 0.045 |
| C9 | | | | | | | 0.30 | 0.14 | 0.03 | | | | | | |
| C10 | | | | 0.43 | 0.20 | 0.04 | | | | | | | −0.68 | 0.26 | 0.01 |
| C14.1 | | | | | | | 0.27 | 0.14 | 0.05 | | | | −0.28 | 0.13 | 0.03 |
| C14.1.OH | | | | | | | 0.34 | 0.14 | 0.01 | | | | −0.41 | 0.13 | 0.00 |
| C14.2 | | | | | | | | | | | | | −0.58 | 0.27 | 0.03 |
| C16 | | | | | | | 0.34 | 0.16 | 0 03 | | | | −0.54 | 0.15 | 0.00 |
| C16.OH | −0.31 | 0.12 | 0.01 | −0.30 | 0.15 | 0.047 | 0.58 | 0.20 | 0.00 | | | | −0.61 | 0.19 | 0.00 |
| C16.1 | | | | | | | 0.40 | 0.18 | 0.03 | | | | −0.52 | 0.17 | 0.00 |
| C16.1.OH | −0.23 | 0.11 | 0.04 | | | | | | | | | | −0.33 | 0.17 | 0.05 |
| C16.2 | | | | | | | | | | | | | −0.60 | 0.21 | 0.00 |
| C16.2.OH | −0.28 | 0.11 | 0.01 | | | | | | | | | | | | |
| C18 | −0.20 | 0.09 | 0.04 | | | | | | | | | | | | |
| C18.1 | | | | | | | | | | | | | −0.45 | 0.22 | 0.04 |
| C18.1.OH | | | | | | | 0.52 | 0.17 | 0.00 | | | | −0.54 | 0.17 | 0.00 |

CD: Core depression, ANX: Anxiety, NVSM: Neurovegetative Symptom of Melancholia: Linear mixed effect models were used to assess the
signilicance of visit by phenotype interaction. Models were adjusted for age, sex, specific drug name (escitalopram/citalopram) and baseline
HRSD₁₇ score. Significance level were set to a = 0.05 not corrected for Bonferroni corrections.

Acylcarnitines Concentrations at Baseline Distinguish Phenotype (+) from Phenotype (−) at 8 Weeks

At 8 week of treatment, NVSM+ has shown significant decrease in the levels of C2, C5-DC-C6-OH, C5-M-DC, C6:C4:1-DC, C7-DC, C10, C14:1, C14:1-OH, C14:2, C16, C16-OH, C16:1, C16:1-OH, C16:2 and C18:1-OH compare to phenotype (−). Significant p-values ranged from <0.05 to <0.001 (Table 12, FIG. 8A-C).

CD+ patients demonstrate significantly higher in the levels of short-chain C5-DC-C6-OH, C5-M-DC, medium-chain C6:C4:1-DC, C7-DC, C9, long-chain C14:1, C14:1-OH, C16, C16-OH, C16:1 acylcarnitines compare to NVSM+. CD+ patients demonstrate significantly lower in the levels of short-chain C2, C3 and C6-C4:1-DC acylcarnitines compare to ANX+. NVSM+ patients demonstrate significantly lower in the levels of short-chain C0, C2, C3 C5-DC-C6-OH, C5-M-DC, medium-chain C6:C4:1-DC, C7-DC, C10, long-chain C14:1, C14:1-OH, C16, C16-OH, C16:1, C16:1-OH, C16:2 and C18:1-OH acylcarnitines compare to ANX+. Significant p-values ranged from <0.05 to <0.001 (Table 12, FIG. 8A-C).

Acylcarnitines Trajectories of Change in Phenotypes from Baseline to Week 8

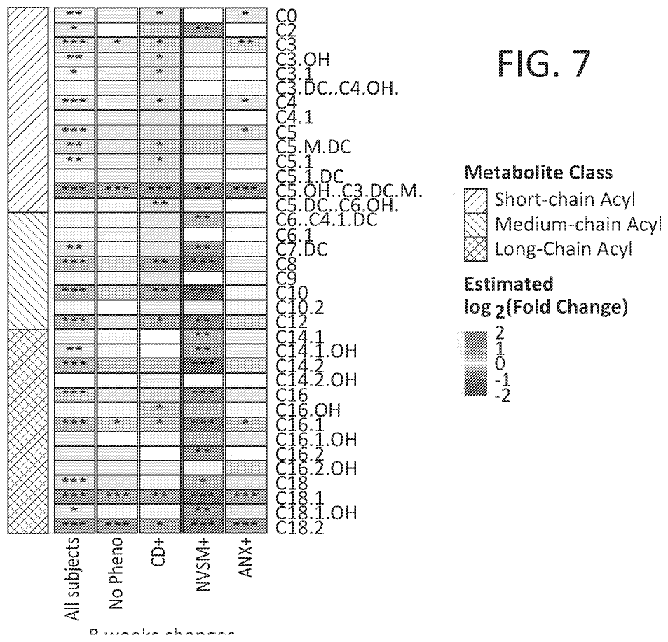

Several short-, medium-, and long-chain acylcarnitines have shown significant changes across the 3 phenotypes over the course of drug therapy. A heatmap (FIG. 7) illustrates key changes in acylcarnitine evaluated after eight weeks of treatment. Exclusively CD+ had significantly increased in levels of 5 short-chains (C3:1, C3-OH, C5-M-DC, C5:1 and C5-DC-C6-OH) and one long-chain (C16-OH) acylcarnitines. Moreover, NVSM+ have significantly decreased in levels C2, 2 medium-(C6-C4:1-DC and C7-DC) and 6 long-chain (C14:1, C14:1-OH, C14:2, C16, C16:2 and C18) acylcarnitines, and C5 is significantly increased only in ANX+. C8, C10, C12 levels significantly decreased in both CD+ and NVSM+, and C0, C3, C4 levels significantly increased in both CD+ and ANX+. Worth noting, all-phenotypes have shown significantly decreased in C5-OH-C3-DC-M, C16:1, C18:1 and C18:2 acylcarnitines levels. Significant p-values ranged from <0.05 to <0.001 (FIG. 7, Table 13).

TABLE 13
8-Week Changes in Acylcarnitines Levels in Response to
SSRI Treatment.

| | | | |
|---|---|---|---|
| Metabolite | Estimate | SE | P-value* |

CD+: baseline - week 8

| | | | |
|---|---|---|---|
| C0 | −0.142149286 | 0.070894166 | 0.045755693 |
| C3 | −0.237864005 | 0.107564113 | 0.0276553 |
| C3.OH | −0.184276419 | 0.081450323 | 0.024304949 |
| C3.1 | −0.202808316 | 0.08611094 | 0.019079254 |
| C4 | −0.230417893 | 0.091969427 | 0.012731914 |
| C5.DC..C6.OH. | −0.219901629 | 0.081976002 | 0.007655889 |
| C5.M.DC | −0.307391656 | 0.155666275 | 0.049087751 |
| C5.OH..C3.DC.M. | 0.595661127 | 0.167082618 | 0.000408523 |
| C5.1 | −0.216309626 | 0.085797064 | 0.012137232 |
| C8 | 0.500655868 | 0.181323952 | 0.006042514 |
| C10 | 0.504864114 | 0.174884625 | 0.004122157 |
| C12 | 0.421863542 | 0.188493737 | 0.025800009 |
| C16.OH | −0.31350854 | 0.131734671 | 0.017827034 |
| C16.1 | 0.252179447 | 0.122746788 | 0.040616302 |
| C18.1 | 0.484856294 | 0.157992422 | 0.002296811 |
| C18.2 | 0.418518698 | 0.170475209 | 0.014522436 |

NVSM+: baseline - week 8

| | | | |
|---|---|---|---|
| C2 | 0.528981203 | 0.186383198 | 0.00480418 |
| C5.OH..C3.DC.M. | 0.677560937 | 0.239427923 | 0.004896248 |
| C6..C4.1.DC. | 0.347708035 | 0.12383032 | 0.005259462 |
| C7.DC | 0.531245273 | 0.171386236 | 0.002099231 |
| C8 | 0.879397821 | 0.259481111 | 0.000777192 |
| C10 | 1.159641521 | 0.249934766 | 4.9117E−06 |
| C12 | 0.771648117 | 0.269743653 | 0.004468521 |
| C14.1 | 0.33666214 | 0.129619295 | 0.009779561 |
| C14.1.OH | 0.405245938 | 0.129000168 | 0.001820578 |
| C14.2 | 0.935129568 | 0.262026601 | 0.000406742 |
| C16 | 0.606857503 | 0.150401912 | 6.66827E−05 |
| C16.1 | 0.830496798 | 0.175695956 | 3.24265E−06 |
| C16.2 | 0.542944732 | 0.20749374 | 0.009243428 |
| C18 | 0.298869619 | 0.1336949 | 0.026039068 |
| C18.1 | 0.952923544 | 0.226419759 | 3.18436E−05 |
| C18.1.OH | 0.501751509 | 0.166415951 | 0.002749193 |
| C18.2 | 0.826280271 | 0.244265983 | 0.000790791 |

ANX+: baseline - week 8

| | | | |
|---|---|---|---|
| C0 | −0.125170826 | 0.060443856 | 0.039206352 |
| C3 | −0.248650357 | 0.092242967 | 0.007398405 |
| C4 | −0.156817868 | 0.077872914 | 0.044944915 |
| C5 | −0.19970477 | 0.09525957 | 0.036821252 |
| C5.OH..C3.DC.M. | 0.570477555 | 0.145946549 | 0.000110726 |
| C16.1 | 0.268671174 | 0.106508446 | 0.012099566 |
| C18.1 | 0.46749925 | 0.138158725 | 0.000792614 |
| C18.2 | 0.525454132 | 0.148767694 | 0.000466074 |

Phenotype(−):baseline - week 8

| | | | |
|---|---|---|---|
| C3 | −0.162064535 | 0.074462054 | 0.030220106 |
| C5.OH..C3.DC.M. | 0.438932939 | 0.116543383 | 0.000191531 |
| C16.1 | 0.190912467 | 0.085437352 | 0.026050668 |
| C18.1 | 0.416695612 | 0.110230554 | 0.000181246 |
| C18.2 | 0.529243979 | 0.118880796 | 1.11783E−05 |

All subjects:baseline - week 8

| | | | |
|---|---|---|---|
| C0 | −0.07913 | 0.026218 | 0.002743 |
| C2 | 0.102976 | 0.049315 | 0.037505 |
| C3 | −0.1935 | 0.039864 | 1.85E−06 |
| C3.OH | −0.07906 | 0.030061 | 0.008941 |
| C3.1 | −0.06434 | 0.031847 | 0.044144 |
| C4 | −0.13179 | 0.033591 | 0.000107 |
| C5 | −0.16184 | 0.040874 | 9.13E−05 |
| C5.M.DC | −0.18625 | 0.058091 | 0.001472 |
| C5.OH..C3.DC.M. | 0.516103 | 0.062741 | 2.97E−15 |
| C5.1 | −0.08683 | 0.031788 | 0.006629 |
| C7.DC | 0.133542 | 0.046162 | 0.004057 |
| C8 | 0.311385 | 0.069383 | 9.59E−06 |
| C10 | 0.332911 | 0.067542 | 1.26E−06 |
| C12 | 0.312513 | 0.072039 | 1.85E−05 |
| C14.1.OH | 0.098561 | 0.034441 | 0.004456 |
| C14.2 | 0.34857 | 0.06981 | 9.18E−07 |
| C16 | 0.222024 | 0.039478 | 3.74E−08 |
| C16.1 | 0.315826 | 0.046561 | 4.63E−11 |
| C18 | 0.121306 | 0.03437 | 0.000474 |
| C18.1 | 0.507753 | 0.059291 | 2.55E−16 |
| C18.1.OH | 0.102464 | 0.043725 | 0.019647 |
| C18.2 | 0.539169 | 0.063779 | 5.95E−16 |

CD: Core depression, ANX: Anxiety, NVSM: Neurovegetative Symptom of Melancholia; Linear mixed effect models were used to assess the significance of visit by Phenotype interaction. *P-value adjusted for age; sex, drug names (escitaloprarn or citalopram) and HRSD-17 score. Significance level were set to α = 0.05 not corrected for Bonferroni corrections.

Baseline to Week Eight Changes in Acylcarnitine Metabolomic Patterns that Differentiate Each Pure Phenotype (CD+; NVSM+; ANX+) from the Others

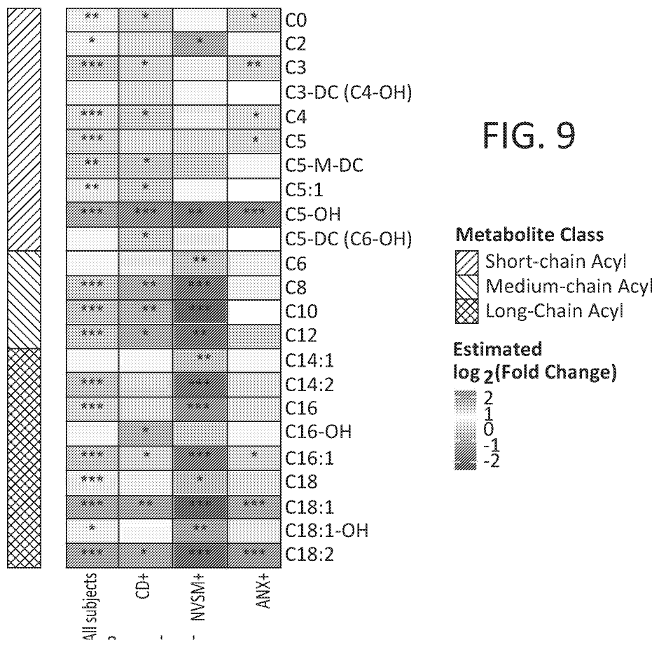

In all subjects, over the eight-week course of SSRI treatment, there were significant increases in the levels of several short-chain acylcarnitines and significant decreases in the levels of several medium and long-chain acylcarnitines (FIG. 7). Different patterns in acylcarnitine changes were noted across the three phenotypes. Patients characterized by the greatest levels of sadness and anhedonis (CD+), demonstrated the significant increases in five short-chain acylcarnitines (C0, C3, C4, C5-M-DC and C5:1). The free (C0) and short-chain acylcarnitines C3, and C4 significantly increased in both CD+ and ANX+. The medium-chain acylcarnitines C8, C10, and C12 significantly decreased in CD+ and NVSM+. Finally, nearly all the long-chain acylcarnitines demonstrated highly significant decreases in the NVSM+ group, but not in the CD+ or ANX+. There were four acylcarnitines, C5-OH, C16:1, C18:1, and C18:2, that significantly decreased in all three phenotypes, perhaps reflecting the effects of exposure to SSRI. Significance of p-values ranged from <0.05 to <0.001. FIG. 9 shows the change of metabolite levels from baseline to eight weeks of SSRI treatment among the three phenotypes and significant p-values (rows represent the acylcarnitine and columns represent the phenotype).

This study is the first to biochemically classified MDD symptoms based on definite acylcarnitines metabolic signatures at baseline, and using escitalopram/citalopram treatment as probes, we distinctly highlighted the uniqueness of these clinical phenotypes (CD, NVSM and ANX). Our results show some acylcarnitines patterns distinguishing between the three phenotypes (+) and phenotype (−) at baseline and after 8 weeks of treatment. Core depression phenotype (+) was associated with significant decrease in short-chain acylcarnitines at baseline and significant increase in the levels of the short-chains acylcarnitines after treatment. Neurovegetative symptom of melancholia phenotype (+) was associated with significant decrease in short-chain acylcarnitines at baseline and significant decrease in medium- and long-chain acylcarnitines after treatment. Anxiety phenotype (+) was associated with significant decrease in short-chain acylcarnitines at baseline and significant increase in the levels of the short-chains acylcarnitines after treatment. Worth noting, patients with no phenotype have no clear AC metabolomic signature. After 8 weeks of treatment, all-phenotypes (+) and (−) have shown significantly decreased in 1 short-chain, 3 long-chain acylcarnitines levels.

Our data at baseline shows that all phenotypes (+) seems to have similar acylcarnitines patterns to phenotype (−). However, the CD+ and ANX+ phenotypes have shown some unique acylcarnitines patterns compare to phenotype (−). Moreover, we found differences between phenotype (+) versus one another. Short-chain C0, C3, C3-OH, C3:1, C4:1, C5:1, C5-DC-C6-OH, C5:1, long-chain C16-OH, C16:1-OH, C16:2-OH and C18 levels are significantly different between CD+ and ANX+. Further, short-chain C3-OH, C3:1, C4:1, C5-DC-C6-OH, C10 and long-chain C16-OH acylcarnitines levels are significantly distinguish between NVSM+ and ANX+. At 8 weeks, the phenotypes of CD+ and ANX+ phenotypes are overlapping. Short-chain C2, C3 and C6-C4:1-DC levels are significantly different in CD+ compare to ANX+. While NVSM+ has shown no statistical differences compared to phenotype (−), it showed statistical differences compare to ANX+.

Looking at acylcarnitines trajectories after 8 week of citalopram/escitalopram, we found decrease in the levels of acylcarnitines C2 and C6:C4:1-DC, C7-DC medium, and C14:1, C14:1-OH, C14:2, C16, C16:2 and C18 in NVSM+. NVSM+ phenotype was defined by loss of sleep and appetite.

CD+ phenotype was defined using HRSD-17 by patients who are sad and anhedonic, and ANX+ phenotype by patients with agitation, anxiety psychological, anxiety somatic and hypochondriasis. It appears that the metabotypes of CD+ and ANX+ phenotypes are overlapping. We found that C0, C3 and C4 levels significantly increased in both phenotypes, and C5 is significantly increased only in ANX+. C0 level was inverse correlated with self-rating depression scale in uremic male HD patients. Moreover, patients with uremia undergoing hemodialysis had decrease in their self-rating depression scale and increased C4 (butyryl-carnitine), C5 (isovaleryl/2-methylbutyryl-carnitine) after L-carnitine treatment.

Our data suggested that acylcarnitines has demonstrated distinct patterns, at baseline and after 8 weeks of treatment, between 3 phenotypes (+) among each other's and compare to phenotype (−). These acylcarnitines findings and relationship with mitochondrial fatty acid β-oxidation provide tools to enable mapping of global biochemical changes in depression, means to characterize the remission and depressed stated and well as ways to map effect of antidepressants on metabolic pathways and networks. Further, these findings reflect changing in mitochondrial function or ATP production in patients with MDD. Moreover, linking acylcarnitines data to genetic data has proven to be a powerful approach to highlight variation in predicting antidepressants response among depressed individuals.

At baseline our data demonstrated unique acylcarnitines signatures that separates phenotypes (+) compare to phenotype (−). Moreover, after 8 weeks of treatment, these acylcarnitines have further stratified these phenotypes by showing unique in short-, medium-, and long-chain acylcarnitines in phenotypes (+). These findings clearly refute the previous criticism to the importance of these phenotypes and their biological associations. This study demonstrates the feasibility of using acylcarnitines as a tool for understanding personal variation in MDD drug response and hence sub-classification within the disease to CD+, NVSM+ and ANX+ patients and linking these changes to mitochondrial fatty acid β-oxidation.

### Example 3

### Clinical

The PReDICT study was a randomized clinical trial that enrolled 344 adults ages 18-65 years with a primary psychiatric diagnosis of MDD without psychotic features. The design and clinical results of the study have been published (Dunlop et al., Trials 13, 106 (2012); Dunlop et al., Am. J. Psychiatry 174, 546-556 (2017)). The Structured Interview for DSM-IV (First et al., Structured Clinical Interview for DSM-IV Axis I Disorders-Padent Edition (SCID-U/P, Version 2.0) (1995)) assessed MDD diagnosis, which was confirmed with a psychiatrist's interview. Patients meeting all eligibility criteria were randomized in a 1:1:1 manner to receive either CBT (delivered in up to 16 one-hour individual sessions), escitalopram, or duloxetine for 12 weeks. One-hundred-fifteen patients were assigned to CBT, of whom 26 had serum samples available for metabolomic analyses at baseline and week 12; these 26 patients are the subjects of the current analysis.

Key inclusion criteria for the trial included no lifetime history of having received treatment for depression (either ≥4 weeks of antidepressant medication at a minimally effective dose or ≥4 sessions of an evidence-based psychotherapy), and fluency in either English or Spanish. At screening, patients had to score ≥18 on the HRSD₁₇ (Hamilton, Br. J. Soc. Clin. Psychol. 6, 278-296 (1967)) and at the baseline randomization visit had to score ≥15. Key exclusion criteria included: a lifetime history of bipolar disorder, psychotic disorder, or dementia; a current significant medical condition that could affect study participation or data interpretation; a diagnosis of obsessive-compulsive disorder, an eating disorder, substance dependence, or dissociative disorder in the 12 months before screening; or substance abuse within the 3 months prior to baseline. The only other psychotropic agents permitted during the trial were sedatives (eszopiclone, zolpidem, zaleplon, melatonin, or diphenhydramine) up to three times per week. The Emory Institutional Review Board and the Grady Hospital Research Oversight Committee approved the study protocol, and all patients provided written informed consent prior to beginning study procedures.

The therapy was delivered in accordance with Beck's protocol-based CBT (Beck et al., Cogniive Therapy ofDepression (1979)) and therapists' fidelity to the protocol was assessed by independent raters at the Beck Institute using the Cognitive Therapy Scale (Young and Beck, Cognitive Therapy Scale: Rating manual (1980)). Raters blinded to treatment assignment assessed depression severity using the HRSD₁₇ at baseline, weeks 1-6, 8, 10, and 12. For the individual patient outcomes, the protocol defined remitters as patients who achieved HRSD₁₇ score ≤7 at both week 10 and 12 (Dunlop et al., Trials 13, 106 (2012)). Consistent with the prior analyses of this dataset (Dunlop et al., Am. J. Psychiatry 174, 548-556 (2017)), outcomes for nonremitters were using percent change in HRSD₁₇ score from baseline to week 12, as follows: Non-remitting responder, 50% reduction, but not meeting remitter criteria; Partial responder: 30-49% reduction; Treatment failure: <30% reduction.

### Laboratory

Metabolomic Profiling Using Absolute IDQ p180 Kit Metabolites were measured with a targeted metabolomics approach using the AbsoluteIDQ p180 Kit (BIOCRATES Life Science AG, Innsbruck, Austria), with an ultra-performance liquid chromatography (UPLC)/MS/MS system [Acquity UPLC (Waters), TQ-S triple quadrupole MS/MS (Waters)]. This procedure provides measurements of up to 186 endogenous metabolites in quantitative mode (amino acids and biogenic amines) and semi-quantitative mode (acylcarnitines, sphingomyelins, phosphatidylcholines and lysophosphatidylcholines across multiple classes). The AbsoluteIDQ p180 kit has been fully validated according to European Medicine Agency Guidelines on bioanalytical method validation. Additionally, the kit plates include an automated technical validation to assure the validity of the run and provide verification of the actual performance of the applied quantitative procedure including instrumental analysis. The technical validation of each analyzed kit plate was performed using MetIDQ software based on results obtained and defined acceptance criteria for blank, zero samples, calibration standards and curves, low/medium/high-level QC samples, and measured signal intensity of internal standards over the plate. De-identified samples were analyzed following the manufacturer's protocol, with metabolomics labs blinded to the clinical data.

Preprocessing of p180 Profiles

The raw metabolomic profiles included 182 metabolite measurements of serum samples. Each assay plate included a set of duplicates obtained by combining approximately 10 μl from the first 76 samples in the study (QC pool duplicates) to allow for appropriate inter-plate abundance scaling based specifically on this cohort of samples (n=24 across all plates). Metabolites with >40% of measurements below the lower limit of detection (LOD) were excluded from the analysis (n=160 metabolites passed QC filters). To adjust for the batch effects, a correction factor for each metabolite in a specific plate was obtained by dividing the metabolite's QC global average by QC average within the plate. Missing values were imputed using each metabolite's LOD/2 value followed by log 2 transformation to obtain a normal distribution of metabolite levels. The presence of multivariate outlier samples was checked by evaluating the squared Mahalanobis distance of samples. Samples were flagged as “outliers” when their Mahalanobis distances exceeded the critical value corresponding to a Bonferroni-corrected threshold (0.05/n, n: number of samples) of the Chi-square distribution with m degrees of freedom (m=160: number of metabolites).

### Data Analysis

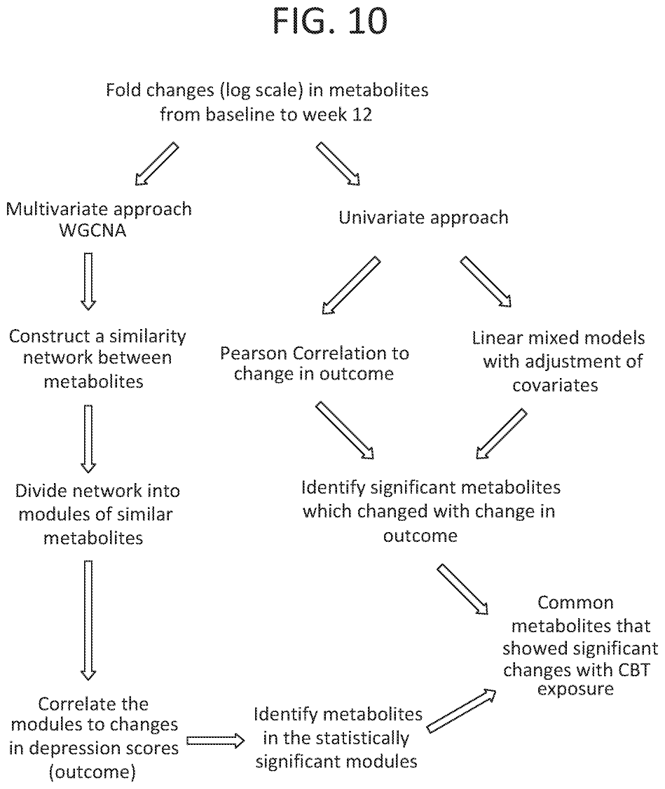

For statistical analysis we adopted a two-pronged approach. Initially, a multivariate “co-expression network” analysis was employed with CBT treated patients to detect clusters (or modules) of metabolites demonstrating similar patterns of perturbations that correlated with changes in depressive symptom scores based on the HRSD₁₇. Univariate analyses were also performed to detect whether the metabolites within or outside the clusters were individually and significantly correlated to the depressive symptom outcome. The traditional univariate analysis method to metabolomic profiling focuses on the individual metabolites; thus, the interactions among metabolites are largely ignored, even though it is appropriate to assume that metabolites play their roles not in isolation but via interactions with each other. Consequently, metabolite “co-expression” analysis is a powerful, multivariate approach to identify groups of perturbed metabolites belonging to same class or pathways. This approach has the additional benefit of alleviating the multiple testing problem (DiLeo et al., PLoS One 6, e26683 (2011)). The workflow of data analysis is presented in FIG. 10.

### Univariate Analysis

To define the association between changes in metabolite levels from baseline to week 12 of CBT treatment and the changes in depressive symptom of total HRSD₁₇ scores over that time, linear mixed effects models were fitted to each metabolite change, adjusting for age and gender and with subjects as a random variable. All p-values were checked for false discovery rates by Benjamini-Hochberg method (Hochberg and Benjamini, Stat. Med. 9, 811-818 (1990)). Correlation between metabolite changes and depressive symptoms changes were also assessed by Pearson's correlation coefficients.

### Co-Expression Network Analyses

Changes in modules of “co-expressed” metabolites were identified using the R package WGCNA (weighted gene co-expression network analysis) (Langfelder and Horvath, BMC Bioinformatics 9, 559 (2008)). Signed and weighted Pearson's correlation networks were constructed with the subject-wise changes of baseline to week 12 metabolite concentrations (in the logarithmic scale). First a weighted adjacency matrix was created based on pairwise Pearson's correlation coefficients between the metabolites. A scale-free topology criterion was used to choose the soft threshold of beta=18 for the correlations as per the WGCNA protocol. The obtained adjacency matrix was used to calculate the topological overlap measure (TOM) for each pair of metabolites log 2 fold changes comparing their adjacencies with all of the other metabolite log 2 fold changes. Densely interconnected groups (or modules) of metabolites were identified by hierarchical clustering using 1-TOM as a distance measure through the use of the dynamic hybrid tree cut algorithm with a deep split of 2 and a minimum cluster size of 3. Each module is summarized by the module eigenvector, which is the first principal component of the metabolite changes across all the subjects. Similar clusters were subsequently merged if the correlation coefficient between the clusters' eigenvectors exceeded 0.75. The association between the resultant modules and the changes in HRSD₁₇ scores was measured by the pairwise Pearson correlation coefficients and presented in a heatmap. In all analyses for this small-scale pilot study an uncorrected p-value threshold of 0.10 was used as the significance cutoff.

### Patient Characteristics

Plasma metabolite data were available at baseline and week 12 from 26 patients. The mean number of therapy sessions attended was 14.0±1.5. Table 14 summarizes the characteristics of the study sample. Four patients (15.4%) were in a chronic depressive episode. Twelve (46.2%) of the sample achieved remission and 7 (26.9%) were classified as treatment failures.

TABLE 14
Patient characteristics

| Measures | Categories | Mean (SD or Percentage) | |
|---|---|---|---|
| No. of patients | | 26 | |
| Age^{a} | | 37:4 | (10:8) |
| Gender^{b} | | | |
| | Female | 16 | (61:5%) |
| | Male | 10 | (38:5%) |
| Response to | | | |
| Therapy^{b} | | | |
| | Remifters | 12 | (46:2%) |
| | Responders (Non-remitting) | 3 | (11:5%) |
| | Partial-Responders | 4 | (15:4%) |
| | Treatment Failures | 7 | (26:9%) |
| HRSD₁₇^{a} | | | |
| | Baseline | 18:6 | (3:1) |
| | Week 12 | 8:7 | (7:2) |

^{a}Mean (SD); ^{b}Mean (percentage)

### Detection of Metabolite “Co-Expression” Modules

To investigate the functional response of the MDD metabolome during receipt of CBT, we adopted a multivariate approach. Using WGCNA methodology we focused on identifying modules (or clusters) of metabolites that showed a similar pattern of change from baseline to week 12. Thus, each module represented metabolite changes (week 12/baseline ratios) in the logarithmic scale. Eight such metabolite modules were identified in which the member metabolites showed statistically significant strong correlations (mean R² ranged between 0.74 and 0.94, all p<0.05) amongst each other in their perturbation patterns, and each module was assigned a unique color. Black, blue, brown, green-yellow, midnight-blue, purple, royal-blue and yellow were the metabolite modules representing 8, 15, 12, 96, 5, 6, 3 and 9 metabolites, respectively. The grey module represented 6 metabolites that could not be assigned to any module. The detected modules were represented by metabolites belonging primarily to the same metabolite class; this may indicate that these metabolites have a functional relationship to each other. Additionally, for each module we identified a “hub” metabolite (also known as a “driver” metabolite) that had the maximum number of connections in the module. The hub metabolites are important, and they merit further investigation because they may influence the function of other metabolites, or even may be significant contributors to the trait of interest. The eight modules, their hub metabolites, and their major metabolite classes are presented in Table 15.

TABLE 15
Characteristics of the co-expression modules of metabolites.

| | Number of | | |
|---|---|---|---|
| | member | | |
| Module | metabolites | Major metabolite classes | Hub metabolite |
| Black | 8 | Amino acids | Asn (Asparagine) |
| Blue | 15 | Medium and long chain | C18:1 (Octadecenoyl-L- |
| | | acylcarnitines | carnitine) |
| Brown | 12 | Lysophosphatidylcholines | lysoPC a C18:1 |
| | | | (Lysophosphatidylcholine |
| | | | acyl C18:1) |
| Green-yellow | 96 | Lipids (phosphatidylcholines | PC aa C42:4 |
| | | and sphingomyelins) and | (Phosphatidylcholine diacyl |
| | | acylcarnitines | C42:4) |
| Midnight-blue | 5 | acylcarnitines | C16:2 |
| | | | (Hexadecadienoylcarnitine) |
| Purple | 6 | Short-chain | acylcarnitines, a-aminoadipic |
| | | | acid |
| C3 (Propionyl- | | | |
| L-carnitine) | | | |
| Royal blue | 3 | sphingomyelins | SM 018:1 (Sphingomyelin |
| | | | C18:1) |
| Yellow | 9 | Branched-chain amino acids, | Val (Valine) |
| | | neurotransmitter-related | |
| | | amino acids, | |

Metabolite Modules that were Associated with Changes in Depressive Symptoms (HRSD₁₇ Scores)

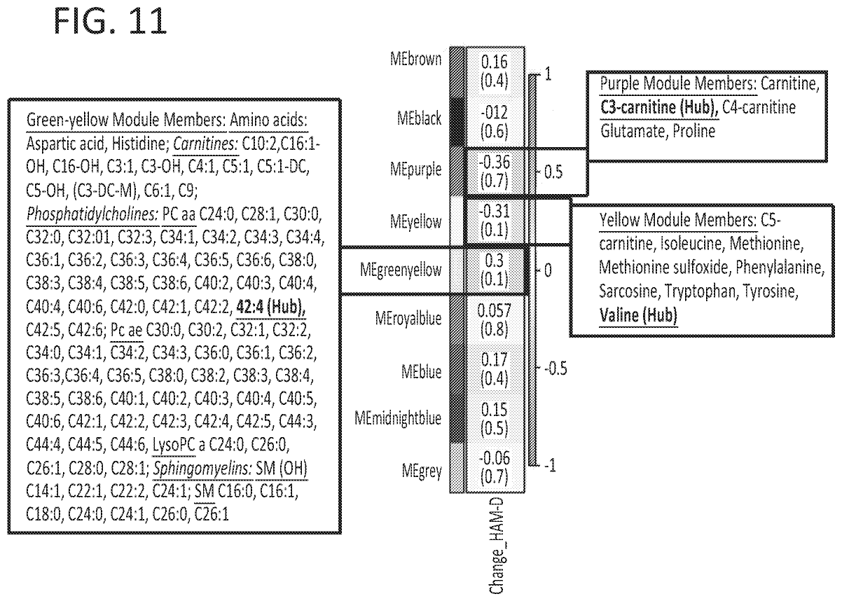

Next, we evaluated the association between the identified metabolite modules and changes in HRSD₁₇ scores from baseline to week 12. Three metabolite modules were found to be significantly associated (R²>0.3, at p<0.1) with changes in the symptom severity cores: (a) the purple module containing the short chain acylcarnitines (C3, C4, and C0), α-minoadipic acid, and the two amino acids, Glutamate and Proline; (b) the yellow module containing the BCAAs, isoleucine and valine, the BCAA-derived C5-carnitine (Isovalerylcarnitine), the neurotransmitter-related amino acids tryptophan, tyrosine, phenylalanine, methionine, methionine-sulfoxide, and the biogenic amine sarcosine; and (c) the green-yellow module containing 96 lipid molecules including the phosphatidylcholines, sphingomyelins, and acylcarnitines. A heatmap showing the correlations between each of the metabolite modules and the changes in HRSD₁₇ scores is presented in FIG. 11. The correlation coefficients were moderate, ranging between 0.31 and 0.36 for each of the three modules.

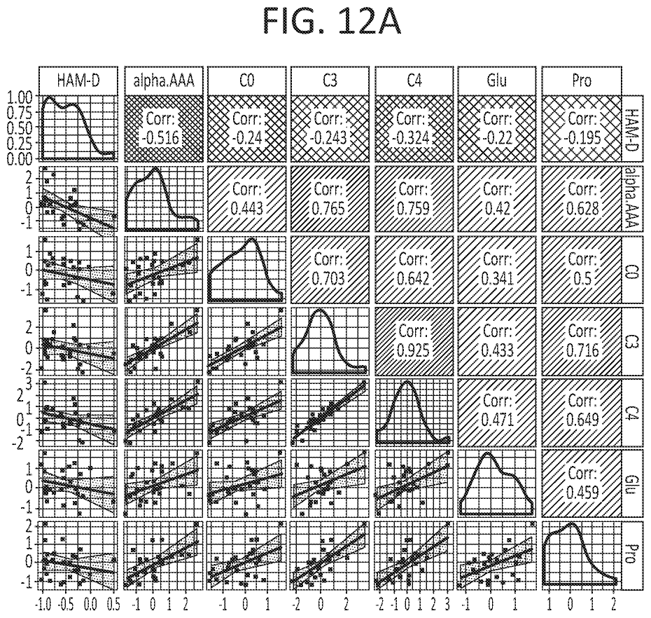

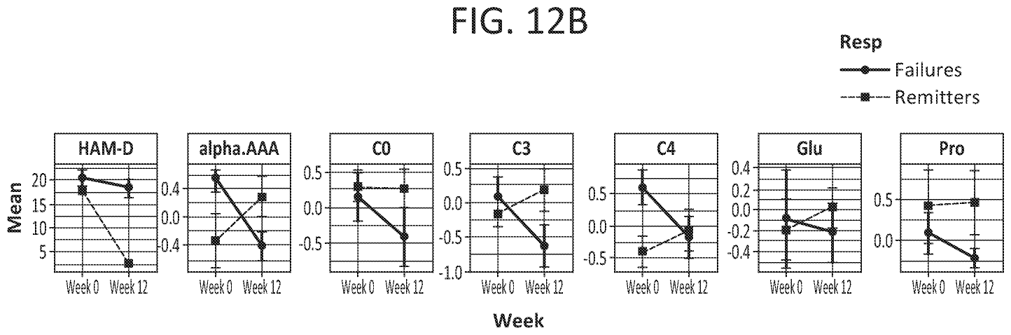

We examined the correlation of each of the metabolite members of the purple, yellow and green-yellow modules to HRSD₁₇ scores. FIG. 12A presents a composite plot of the correlations between the changes of each member-metabolite of the purple module to each other and also to HRSD₁₇ changes. Each of the metabolite changes was negatively associated with changes in HRSD₁₇, but they were positively correlated to each other. α-aminoadipic acid showed the strongest correlation to HRSD₁₇ changes (R²=−0.52, p<7E-06). The BCAA-derived C3-(propionyl) and C4-(butyryl) carnitines were strongly correlated to each other (R²=0.93, p<1.3×10⁻¹¹) as well as to α-aminoadipic acid. C3-carnitine was the hub metabolite in this module.

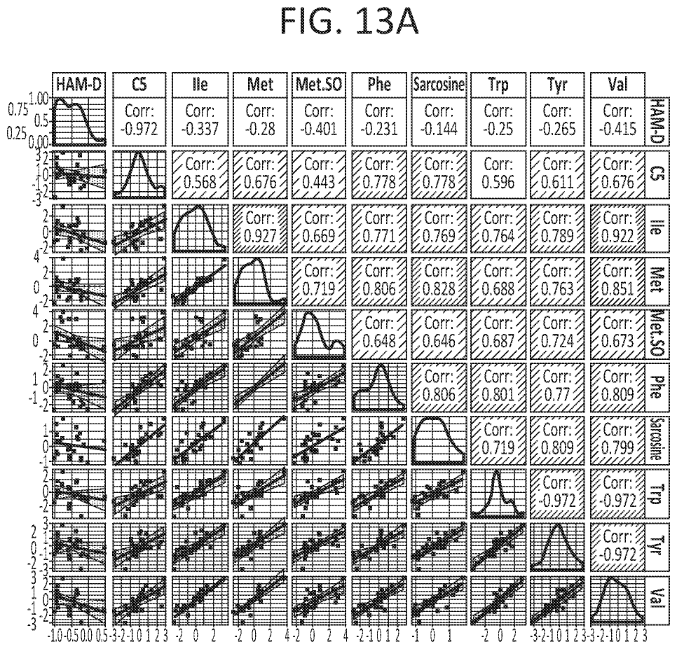

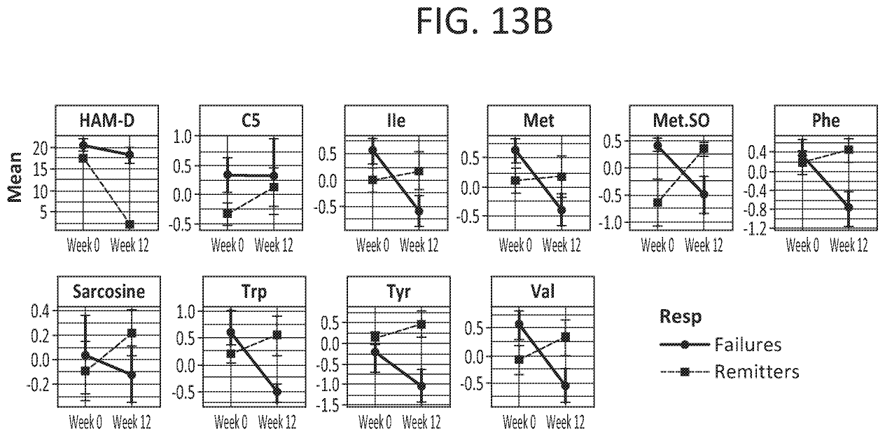

FIG. 13A presents a similar composite figure for the yellow module. All members were negatively correlated to HRSD₁₇ changes, with the BCAA valine and the known oxidative stress biomarker methionine sulfoxide perturbations showing the strongest correlations with HRSD₁₇ changes (R²<−0.40, p<0.05). All the yellow module metabolites showed strong positive correlations with each other (mean R²=0.75, range 0.44-0.93, all p<0.02) in their perturbation patterns. Notable were the strong positive correlations between the branched chain amino acids and methionine, sarcosine, and the neurotransmitter related amino acids (phenylalanine, tryptophan and tyrosine) indicating that they may be functionally related. Valine was the hub metabolite for this module.

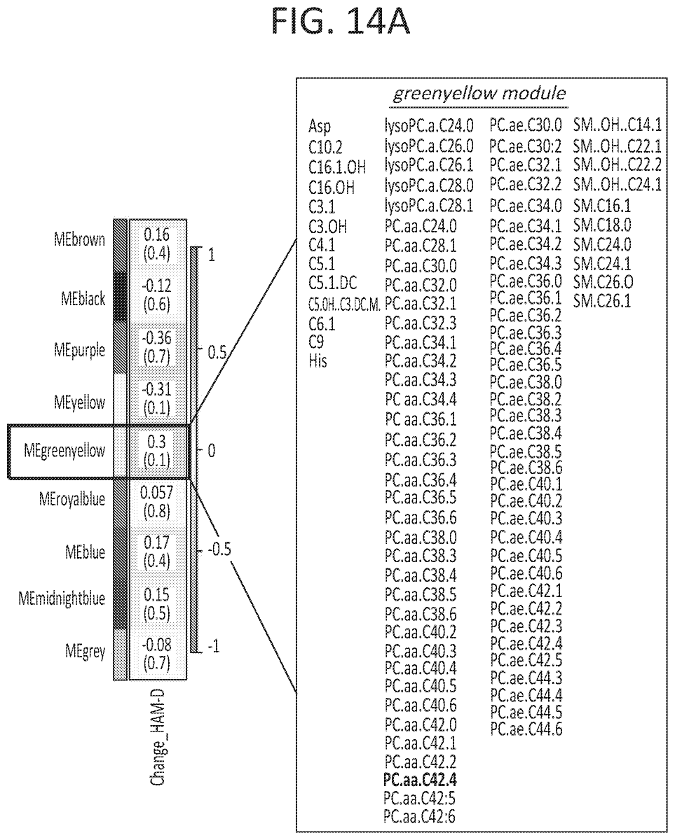

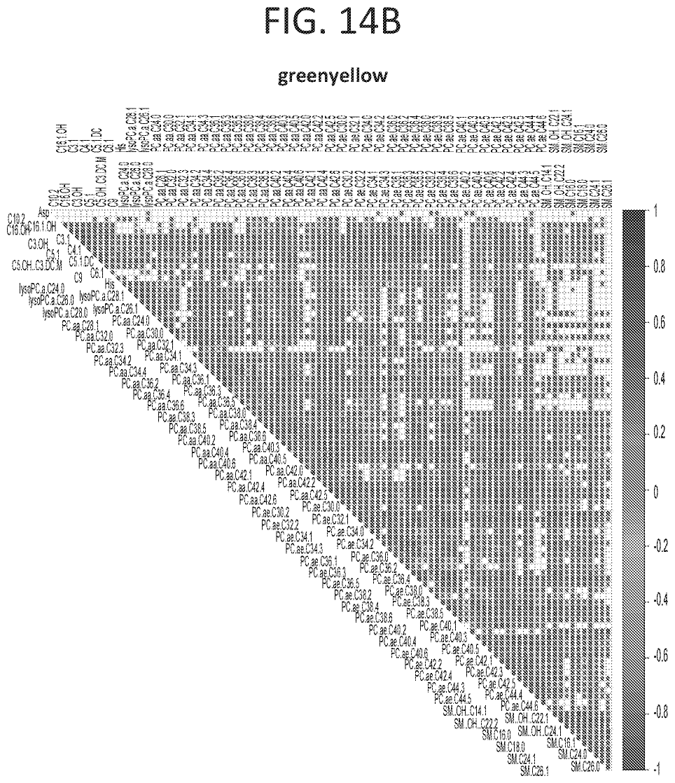

The members of the 96 lipids-containing green-yellow module also showed strong correlations amongst each other and also to HRSD₁₇ changes. Unlike the amino acids and the short chain acylcarnitines from the purple and yellow modules, these lipid molecules' changes were positively associated with HRSD₁₇ changes and also to each other (FIG. 14A-B). In univariate linear mixed model analysis, the log 2 fold changes of the α-aminoadipic acid, the branched chain amino acids, isoleucine and valine, methionine-sulfoxide and several of the phosphatidylcholines, containing long chain fatty acids, from the green-yellow module, were found to be significantly associated (p<0.10) with the changes in HRSD₁₇ scores after adjusting for age and sex. These findings highlight the potential involvements of mitochondral energy metabolism and lipids in the response of depressed patients receiving CBT. Table 17 depicts the list of metabolites that were significant in univaiate models and their associations with depressive symptom scores (HRSD₁₇).

To maximize our ability to detect the effects of treatment outcomes, we plotted the mean trajectories of the metabolites among the CBT remitters (N=12) and the treatment failures (N=7), leaving out the patients with intermediate outcomes, consistent with the approach used in other biomarker studies (Dunlop et al., Am. J. Psychiatry 174, 533-545 (2017); Vadodaria et al., Mol. Psychiatry 24, 795-807 (2019)). There were interesting trends among the metabolites in the purple, yellow, and green-yellow modules. In the green-yellow module, consisting of lipids—75% of the phosphatidylcholines were higher at baseline in the remitters compared to the treatment failures (FIG. 14B), with some of them being statistically significant at p<0.10 (PC aa-C30:0, C34:1, C36:2, C36:3, PC ae-C36:0, C38:2). In the purple (FIG. 12B) and yellow (FIG. 13B) modules, consisting mostly of amino acids including BCAAs and the short-chain acylcarnitines, the remitters mostly had lower baseline levels but showed an upward trend in their metabolite trajectories from baseline to week 12 while the treatment failures all trended to decrease to lower levels post therapy. The limited sample size, however, precluded detection of statistical significance to these differences in trajectories.

TABLE 17
Association of metabolite changes to HRSD₁₇ changes from baseline to week 12 upon
exposure to CBT, by univariate models.

| | Pearson | correlation | Linear mixed | | mode | Module- |
|---|---|---|---|---|---|---|
| Metabolite | Corr. Coeff. | p-value | Coeff. Estimate | | p-value | membership |
| a-aminoadipic acid | −0.5164 | 0.0069 | −0.2 | (−0.3, −0.1) | 0.0093 | Purple |
| Isoleucine | −0.3372 | 0.0921 | −0.1 | (−0.2, 0.0) | 0.0812 | Yellow |
| Valine | −0.4147 | 0.0351 | −0.1 | (−0.2, −0.0) | 0.0367 | Yellow |
| Methionine Sulfoxide | −0.4009 | 0.0424 | −0.1 | (−0.2, 0.0) | 0.0512 | Yellow |
| Hexenoylcarnitine (C6:1) | 0.3445 | 0.0848 | | | | Green-yellow |
| aLPC a C24 | 0.4601 | 0.0180 | 0.1 | (0.0, 0.3) | 0.0236 | Green-yellow |
| LPC a C26 | 0.4021 | 0.0417 | 0.1 | (0.0, 0.2) | 0.0510 | Green-yellow |
| LPC a 028 | 0.3666 | 0.0655 | 0.1 | (0.0, 0.3) | 0.0730 | Green-yellow |
| bPC aa 034:1 | 0.4041 | 0.0406 | 0.2 | (0.0, 0.3) | 0.0584 | Green-yellow |
| PC aa C34:3 | 0.3897 | 0.0491 | 0.1 | (0.0, 0.2) | 0.0724 | Green-yellow |
| PC aa C36:1 | 0.3501 | 0.0796 | | | | Green-yellow |
| PC aa 036:2 | 0.4238 | 0.0309 | 0.2 | (0.0, 0.3) | 0.0431 | Green-yellow |
| PC aa C36:3 | 0.4388 | 0.0249 | 0.2 | (0.0, 0.3) | 0.0369 | Green-yellow |
| PC aa C38:3 | 0.3748 | 0.0592 | 0.1 | (0.0, 0.2) | 0.0829 | Green-yellow |
| PC aa C38:5 | 0.3479 | 0.0816 | | | | Green-yellow |
| PC aa C40:2 | 0.4232 | 0.0312 | 0.2 | (0.0, 0.3) | 0.0419 | Green-yellow |
| PC aa C40:3 | 0.3589 | 0.0718 | 0.1 | .0.0, 0.3) | 0.0976 | Green-yellow |
| PC aa C40:4 | 0.3532 | 0.0767 | | | | Green-yellow |
| PC aa C40:5 | 0.3363 | 0.0930 | | | | Green-yellow |
| PC aa C42:6 | 0.3401 | 0.0891 | | | | Green-yellow |
| cPC ae C30:0 | 0.4160 | 0.0346 | 0.2 | (0.0, 0.3) | 0.0495 | Green-yellow |
| PC ae C34:1 | 0.3875 | 0.0505 | 0.1 | (0.0, 0.3) | 0.0702 | Green-yellow |
| PC ae C38:2 | 0.5777 | 0.0020 | 0.3 | (0.1, 0.4) | 0.0033 | Green-yellow |
| PC ae C38:3 | 0.3817 | 0.0543 | 0.1 | (0.0, 0.3) | 0.0749 | Green-yellow |
| PC ae C40:3 | 0.3604 | 0.0705 | 0.2 | (0.0, 0.3) | 0.0952 | Green-yellow |
| PC ae C42:1 | 0.4554 | 0.0194 | 0.1 | (0.0, 0.3) | 0.0330 | Green-yellow |
| PC ae C42:2 | 0.3621 | 0.0691 | | | | Green-yellow |
| PC ae C42:4 | 0.3299 | 0.0998 | | | | Green-yellow |
| Octadecadienylcarnitine | 0.3572 | 0.0732 | | | | Blue |
| (C18:2) | | | | | | |

According to common lipid nomenclature, aLPC stands for lysophosphatidylcholine with a single fatty acid chain, bPC aa stands for Phosphatidylcholine diacyl (two fatty acid chains) and cPC ae stands for Phosphatidylcholine acyl-alkyl. The lipid species are described as CX:Y where X is the length of the carbon chain C, Y is the number of double bonds: “a” means the acyl chain is attached via an ester bond to the backbone while “e” means the attachment is via an ether bond.

Using liquid-chromatography coupled to mass-spectrometry analyses, we examined the biochemical changes that occurred in the plasma of depressed outpatients completing a course of CBT. Changes in several metabolite modules, containing primarily short-chain acylcarnitines and α-aminoadipic acid (purple module) as well as branched-chain and neurotransmitter-related amino acids (yellow module) and lipids (green-yellow module), were significantly associated with changes in depressive symptom severity over the 12-weeks of CBT treatment. The metabolites within each module were highly correlated, and therefore it is likely that the similarity in their perturbations stemmed from their functional relatedness or being members of the same affected pathways.

The lipid perturbations, especially those of the phosphatidylcholines (consisting of either diacyl or alkyl-acyl moieties) showed positive correlations to the changes in HRSD₁₇ scores. Phosphatidylcholines are a large class of lipid molecules commonly known as the glycerophospholipids. They have important functions in membrane stability, permeability, and signaling.

These results, showing that the metabolites may serve as state markers of depression, received tentative support from our exploratory contrast of the differential trajectories of the changes in metabolites between the patients with the clearest treatment outcomes: remitters versus treatment failures. The BCAAs, their catabolic byproducts, the short chain acylcarnitines, the lysine metabolite α-aminoadipic acid, and the aromatic amino acids (phenylalanine, tyrosine and tryptophan) were present at comparatively higher levels at baseline in the treatment failures compared to the remitters. These findings suggest that metabolic wellbeing may be an important factor contributing to CBT response. Interestingly, there was a general downward trend in the trajectories of these metabolites over the course of treatment among the CBT treatment failures, whereas in the remitters they all exhibited stable or upward trajectories.

In summary, this evaluation assessed 180 metabolites from the Biocrates Absolute p180 kit that clustered into 8 “co-expression modules” based on their propensities to change over 12 weeks of treatment with CBT. The results were largely confirmed by additional univariate analyses of the individual metabolites in the co-expression analyses. Specifically, BCAAs, methionine sulfoxide, α-aminoadipic acid, and multiple phosphatidylcholines were all altered in association with changes in the HRSD₁₇ scores at a level that equaled or exceeded a correlation coefficient of 0.4. Hence, these metabolites represent markers for the depressed state, and perhaps may act as moderators or mediators for improvement from depression. These results will be useful in comparing and contrasting metabolomic changes that occur during and after treatment with antidepressant medication, and perhaps serving as a biomarker to inform treatment selection for MDD patients. In addition, metabolomic profiles in medication-free patients have utility as a biomarker for impending depressive relapse during long-term follow-up studies.

### Example 4

Study Design and Participants We used samples from the Mayo Clinic NIH-Pharmacogenomics Research Network-Antidepressant Pharmacogenomics Medication Study (PGRN-AMPS) which recruited a total of 803 MDD patients (Mrazek et al., J. Clin. Psychopharmacol. 34, 313-317 (2014)). Patient selection, symptomatic evaluation, and blood sample collection for the PGRN-AMPS clinical trial have been described elsewhere (Schiepers et al., Prog. Neuro-Psychopharmacol. Biol. Psychiatry 29, 201-217 (2005); Gupta et al., Mol. Psychiatry 21, 1717-1725 (2016); Mrazek et al., J. Clin. Psychopharmacol. 34, 313-317 (2014); Ji et al., Br. J. Clin. Pharmacol. 78, 373-383 (2014)). Briefly, MDD patients were required to have a baseline HRSD₁₇ score ≥14, and all patients who completed 8 weeks of treatment (n=290) were treated with one of the two SSRIs, citalopram or escitalopram. Depressive symptoms were assessed with HRSD₁₇ at baseline, week 4, and week 8 of SSRI treatment. Blood samples were collected at these same time points.

The HRSD₁₇ was used to ascribe “response”—defined as at least 50% reduction in the total score from baseline to exit; “remission”—an exit HRSD₁₇ score of 7 or less; and “complete-non-response”—less than 30% reduction in the HRSD₁₇ total score from baseline to exit (Mrazek et al., J. Clin. Psychopharmacol. 34, 313-317 (2014)). Genome-wide association studies for plasma concentrations of the SSRIs and metabolite levels (Ji et al., Br. J. Clin. Pharmacol. 78, 373-383 (2014)) and for response (Ji et al., Pharm. J. 13, 456-463 (2013)) in this trial have been published previously. The trial was designed as a parallel to the large National Institute of Mental Health—funded “the Sequenced Treatment Alternatives to Relieve Depression” (STAR*D) clinical trial (Rush et al., N. Engl. J. Med. 354, 1231-1242 (2006)) for the purpose of replication of the identified genetic markers.

### Metabolomic Profiling

A targeted, liquid chromatography-electrochemical coulometric array (LCECA) metabolomics platform (Matson et al., Clin. Chem. 30, 1477-1488 (1984)) was used to assay metabolites in plasma samples from the three time points, baseline, 4 weeks, and 8 weeks. This platform was used to identify and quantify 31 neurotransmitter-related metabolites (against standards) primarily from the TRP, tyrosine, and tocopherol pathways, including serotonin. A list of the metabolites that were quantitatively measured using this platform is presented in Table 18.

TABLE 18
List of metabolites and pathways analyzed in the study

| Metabolite by pathways | Abbreviation |
|---|---|
| Tryptophan | |
| 3-hydroxykynurenine | 3OHKY |
| 5-hydroxyindoleacetic acid | 5HIAA |
| 5-hydroxytryptophan | 5HTP |
| Indole-3-acetic acid | I3AA |
| Kynurenine | KYN |
| Serotonin | 5HT |
| Tryptophan | TRP |
| Tyrosine | |
| 4-hydroxyphenylacetic acid | 4HPAC |
| 4-hydroxyphenyllacetic acid | 4HPLA |
| Homogentisic acid | HGA |
| Homovanillic add | HVA |
| Methoxy-hydroxyphenyl glycol | MHPG |
| Tyrosine | TYR |
| Vanillylmandelic acid | VMA |
| Tocopherol | |
| α-tocopherol | ATOCO |
| δ-tocopherol | DTOCO |
| +65-tocopherol | GTOCO |
| Phenylalanine/tyrosine | |
| 4-hydroxybenzoic acid | 4HBAC |
| Purine | |
| Guanine | G |
| Guanosine | GR |
| Hypoxanthine | HX |
| Uric acid | URIC |
| Xanthine | XAN |
| Paraxanthine | PXAN |
| Xanthosine | XANTH |
| 1 Carbon + GSH | |
| Methionine | MET |
| Cysteine | CYS |
| Other | |
| Salicylate | SA |
| Alpha-Methyltryptophan | AMTRP |
| Indole-3-propionic add | I3PA |
| Theophylline | Theophylline |

### Analysis Method

The long-gradient LCECA method used for this analysis can resolve compounds at picogram levels through electrochemical detection (resulting from oxidation or reduction reactions) including multiple markers of oxidative stress and protection. This method utilizes a 120-min gradient from (0%) organic modifier with an ion-pairing agent (i.e., pentane sulfonic acid) to a highly organic mobile phase with methanol (80%)/isopropanol (10%)/acetonitrile (10%). An array of 16 serial coulometric electrochemical detectors is set at incremental potentials from 0 to 900 mV, responding to oxidizable compounds such as tocopherol in lower potential sensors and higher oxidation potential compounds such as hypoxanthine in the higher potential channels.

### Analysis Sequence and Data Output

At the time of preparation, a pool was created from small aliquots of each sample in the study, which was then treated identically to a sample. All of these assays were executed in sequences that included mixed standard, five samples, pool, five samples, mixed standard, and so on and so forth. In this study, all sample run orders were randomized. The sequences decreased possible analytical artifacts during further data processing. Data were time normalized to a pool at the midpoint of the study, aligning major peaks to 0.5 s and minor peaks to 0.5-2 s. Details on the LCECA methods are described in previously published work (Kaddurah-Daouk et al., Transl. Psychiatry 1, e26 (2011); Ji et al., Pharm. J. 13, 456-463 (2013); Benjamini & Hochberg, J. R. Stat. Soc. Ser. B (Methodol.) 57, 289-300 (1995); Pons & Latapy, In Proc. 20th International Conference on Computer and Information Sciences, 284-293 (2005); von Elm et al., Ann. Intern. Med. 147, 573-577 (2007); Howie et al., PLoS Genet. 5, e1000529 (2009); Delaneau et al., Nat. Methods 10, 5-6 (2013); Chang et al., GigaScience 4, 7 (2015); Golino & Epskamp, PLoS ONE 12, e0174035 (2017)).

### Data Analysis

All data preprocessing and analysis were performed with R (version 3.4.2) and Bioconductor (version 3.3) statistical packages.

### Preprocessing

This study's data extraction protocol followed the STORBE guidelines (von Elm et al., Ann. Intern. Med. 147, 573-577 (2007)). All metabolite data were first checked for missing values (none were detected at >20% missing abundances) and were subjected to imputation by the k-nearest neighbor algorithm (Do et al., Metabol.: Off J. Metabol. Soc. 14, 128 (2018)). Data were then log 2.

### Univariate Analysis

To define the effect of drug exposure over 4 weeks and 8 weeks of treatment, linear mixed effects models (using the R package nlme (Pinheiro et al., The Nlme Package: Linear and Nonlinear Mixed Effects Models, R Version 3 6 (2012))) were fitted on each metabolite adjusting for age, gender, and HRSD₁₇ scores at baseline with subjects as random variable. Analyses were conducted separately for 4 and 8 weeks. Linear mixed effects models were also used to determine associations between the changes in metabolites and changes in HRSD₁₇ over time, with age and gender as covariates, and using subjects as random variable. All p-values were used to calculate the false discovery rates by Benjamini-Hochberg method (Benjamini & Hochberg, J. R. Stat. Soc. B Met. 57, 289-300 (1995)), and a cutoff point of 10% was used. A two-step regression strategy was used to find metabolites with significant temporal changes and significant differences between responders and nonresponders using the maSigPro library in R (Conesa et al., Bioinformatics 22, 1096-1102 (2006)). First, a least-squared technique was employed to identify differential metabolites in a global regression model, using dummy variables for experimental groups. Second, stepwise regression was applied to select variables that differed between the experimental groups and find significantly different metabolite profiles between the groups.

Partial Correlation Networks with Cluster Subgraph Analysis

The relationship between metabolites in a complex disease setting can be represented in terms of partial correlation networks, where each node represents a metabolite and each edge between two metabolites represents that two variables are not independent after conditioning on all variables in the dataset. These edges have a weight, edge weights, which are the partial correlation coefficients. Here, we estimated the partial correlation matrix for all of the metabolites using the least absolute shrinkage and selection parameter (LASSO) to obtain the sparse inverse covariance matrix to avoid overfitting and spurious correlations. Thus, it can be reasonably expected that the regularized partial correlation networks will provide accurate estimates of the underlying relationships between the metabolites in metabolic pathways and reactions. The LASSO regularization parameter was set via EBIC or Extended Bayesian Information criterion (Golino & Epskamp, PLoS ONE 12, e0174035 (2017)). Finally, the walktrap algorithm, which is based on random walks to capture duster structures in a network, is used to identify clusters of strongly interacting metabolites (Pons & Latapy, In Proc. 20th International Conference on Computer and lnformadon Sciences, 284-293 (2005)). The final network with duster subgraphs is formed by the median pairwise partial correlations over 1000 bootstrap estimations and plotted using the Fruchterman-Reingold layout. We further included the HRSD₁₇ scores in our partial correlation network models to perform differential network analysis. The overall statistical impact of HRSD₁₇ scores on the metabolite interactions was calculated based on measuring structure invariance between two networks, high HRSD₁₇ and low HRSD₁₇ networks, constructed using a median split of the variable. Permutation tests were used to determine the significance of structure and edge invariances between the two networks (van Borkulo et al., Network Comparison Test: statistical comparison of two networks based on three invariance measures (R package Version 2.0.1) (2016)). The metabolite-metabolite partial correlations that were of differential strength between networks of high and low HRSD₁₇ networks were further validated for significant interaction effects through linear regression analysis.

Candidate Metabolic Trait GWAS with HGA/GR and MET/TYR Ratios

For 288 of the 290 subjects in this study we had genotype data for the Illumina human 610-Quad BeadChips (Illumina, San Diego, Calif., USA) available, as described previously (Gupta et al., Mol. Psychiatry 21, 1717-1725 (2016); Ji et al., Pharm. J. 13, 456-463 (2013)). Genotype QC using PLINK and imputation followed standard protocols. Briefly, raw genotype data were filtered for variants with call rate <5%, minor allele frequency (MAF)<5%, and Hardy-Weinberg equilibrium HWE p<1×10⁻⁵ (Chang et al., GigaScience 4, 7 (2015)). The data was then subjected to prephasing using SHAPEIT2 (ver. 2.12) (Delaneau et al., Nat. Methods 10, 5-6 (2013)), followed by imputation with IMPUTE2 (ver. 2.3.2) (Howie et al., PLoS Genet. 5, e1000529 (2009)) using 1000 genomes phase 3 version 556 haplotypes as a reference. Post imputation QC included filtering variants for IMPUTE info score <0.5, call rate and MAF <5%, and HWE p <1×10⁻⁵, resulting in a final set of 5.55 mio SNPs with 99.14% genotyping rate. To remove any potential for spurious associations due to population stratification, we used a set of about 100,000 SNPs pruned for the LD structure and retrieved the first five principal component eigenvectors (PCs). Metabolite data for the HGA/GR and Met/TYR ratios were log transformed, centered to zero mean, and scaled to unit variance. In addition, for candidate GWAS, we excluded values that were more than 4 standard deviations from the mean. We then performed GWAS for HGA/GR and MET/TYR at each time point while adjusting for age, sex, and PCs 1-5. We reran the GWAS additionally adjusting for HDRS₁₇ scores at each time point to eliminate the effects linked to depression severity.

### Patient Characteristics

Plasma metabolite data were available from 290 MDD patients. The average age of the patient cohort was 39.8 (±13.1) years. Females comprised of 66% of the study cohort, while males were at 34%. The response rate to the drug, based on HRSD₁₇ scores, was 69.3% after 8 weeks, compared with 30.7% who were classified as nonresponders for this study. The depressive status of the patients, as determined by the HRSD₁₇ scores, decreased over time with the drug treatment, from an average of 21.9 (±4.9) at baseline to 11.6 (±6.4) at week 4 and 8.6 (t5.5) at week 8. Demographic and clinical characteristics are detailed in Table 19.

TABLE 19
Demographics and clinical features of study sample

| Variables | Total (N = 290) | |
|---|---|---|
| Age | 39.8 | (13.1)^{a} |
| Gender | | |
| F | 191 | (65.9%)^{b} |
| M | 99 | (34.1%)^{b} |
| Treatment | | |
| Citalopram | 120 | (41.4%)^{b} |
| Escitalopram | 170 | (58.6%)^{b} |
| Response to Drug | | |
| No | 89 | (30.7%)^{b} |
| Yes | 201 | (69.3%)^{b} |
| HRSD₁₇ | | |
| Baseline | 21.9 | (4.9)^{a} |
| W4 | 11.6 | (6.4)^{a} |
| W8 | 8.6 | (5.5)^{a} |

^{a}Mean (std dev); ^{b}Count (Percentage)

Metabolite Changes at Weeks 4 and 8 Compared with Baseline, in Response to the Drug.

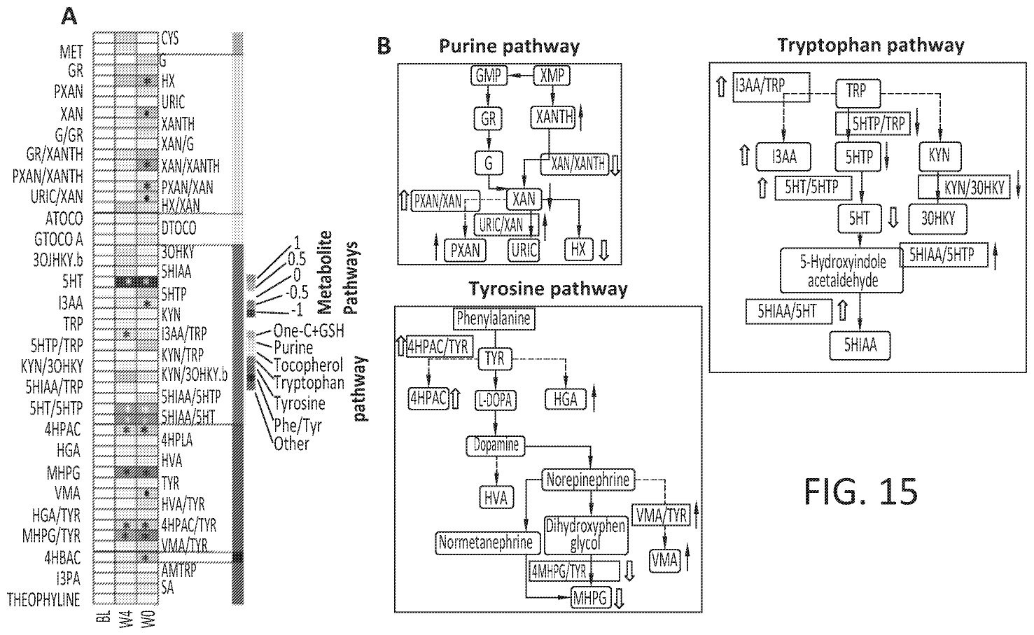

Several metabolites in the purine, tryptophan, and tyrosine pathways changed, following 4 weeks of drug therapy. However, perturbations in the metabolite levels were in general, greater and more significant after 8 weeks of treatment (Tables 20-21). FIG. 15 illustrates changes within key pathways evaluated after 8 weeks of treatment.

TABLE 20
Significantly changing metabolites at unadjusted p <0.05 at week 4 and/or week 8.

| | | | | Linear mixed model | | | |
|---|---|---|---|---|---|---|---|
| | | Fold change^{a} | | BL^{b} vs Week4 | | BL vs Week8 | |
| | | Week4/B | Week8/B | | Adj p- | | Adj p- |
| Pathway | Metabolites | L | L | p-value | value | p-value | value^{c} |
| 1C+ reaox | CYS | −0.15 | −0.05 | 0.0488 | 1 | 0.4959 | 1 |
| 1C+ reciox | MET | −0.1 | 0.02 | 0.1922 | 1 | 0.7995 | 1 |
| Purine | G | −0.01 | −0.14 | 0.8894 | 1 | 0.0498 | 1 |
| Purine | GR | −0.12 | −0.11 | 0.1413 | 1 | 0.1714 | 1 |
| Purine | HX | −0.19 | −0.25 | 0.0106 | 0.575 | 0.0001 | 0.004 |
| Purine | PXAN | 0.03 | 0.15 | 0.6199 | 1 | 0.0034 | 0.183 |
| Purine | URIC | −0.01 | 0.02 | 0.7759 | 1 | 0.654 | 1 |
| Purine | XAN | −0.04 | −0.24 | 0.6068 | 1 | 0.0016 | 0.089 |
| Purine | XANTH | 0.08 | 0.21 | 0.4077 | 1 | 0.0088 | 0.473 |
| Purine | G_by_GR | 0.01 | −0.12 | 0.8847 | 1 | 0.0898 | 1 |
| Purine | XAN_by_G | 0 | 0.07 | 0.9773 | 1 | 0.3327 | 1 |
| Purine | GR_by_XANTH | −0.11 | −0.24 | 0.2353 | 1 | 0.0035 | 0.191 |
| Purine | XAN_by_XANTH | −0.09 | −0.31 | 0.3345 | 1 | 0.0002 | 0.013 |
| Purine | PXAN_by_XANTH | −0.03 | −0.03 | 0.6503 | 1 | 0.6374 | 1 |
| Purine | PXAN_by_XAN | 0.04 | 0.24 | 0.4796 | 1 | 0 | 0.002 |
| Purine | URIC_by_XAN | 0.03 | 0.24 | 0.6675 | 1 | 0.0017 | 0.092 |
| Purine | HX_by_XAN | −0.17 | −0.12 | 0.023 | 1 | 0.0704 | 1 |
| Tocopherol | ATOCO | 0.08 | 0.14 | 0.3296 | 1 | 0.0617 | 1 |
| Tocopherol | DTOCO | −0.02 | 0.09 | 0.755 | 1 | 0.2212 | 1 |
| Tocopherol | GTOCO.A | 0.05 | 0.17 | 0.523 | 1 | 0.0246 | 1 |
| Tryptophan | 3OHKY | −0.12 | 0.08 | 0.0497 | 1 | 0.1318 | 1 |
| Tryptophan | 3OHKY.b | 0.19 | 0.12 | 0.0127 | 0.685 | 0.0972 | 1 |
| Tryptophan | 5HIAA | −0.1 | 0.03 | 0.1164 | 1 | 0.5725 | 1 |
| Tryptophan | 5HT | −1.24 | −1.28 | 0 | 0 | 0 | 0 |
| Tryptophan | 5HTP | −0.05 | −0.18 | 0.5109 | 1 | 0.0229 | 1 |
| Tryptophan | I3AA | 0.14 | 0.18 | 0.0108 | 0.581 | 0.0004 | 0.024 |
| Tryptophan | KYN | −0.05 | 0.05 | 0.332 | 1 | 0.4253 | 1 |
| Tryptophan | TRP | −0.07 | 0.07 | 0.3114 | 1 | 0.3155 | 1 |
| Tryptophan | I3AA_by_TRP | 0.17 | 0.15 | 0.0007 | 0.038 | 0.0029 | 0.159 |
| Tryptophan | 5HTP_by_TRP | −0.04 | −0.19 | 0.5655 | 1 | 0.0191 | 1 |
| Tryptophan | KYN_by_TRP | 0 | 0 | 0.9384 | 1 | 0.9743 | 1 |
| Tryptophan | KYN_by_3OHKY | 0.1 | −0.05 | 0.1345 | 1 | 0.4609 | 1 |
| Tryptophan | 5HIAA_by_TRP | −0.06 | 0 | 0.3155 | 1 | 0.9979 | 1 |
| Tryptophan | 5HIAA_by_5HTP | 0.03 | 0.19 | 0.6782 | 1 | 0.0181 | 0.979 |
| Tryptophan | 5HT_by_5HTP | 0.61 | 0.52 | 0 | 0 | 0 | 0 |
| Tryptophan | 5H1AA_by_5HT | 1.15 | 1.23 | 0 | 0 | 0 | 0 |
| Tyrosine | 4HPAC | 0.22 | 0.31 | 0.0001 | 0.005 | 0 | 0 |
| Tyrosine | 4HPLA | −0.03 | 0.07 | 0.4622 | 1 | 0.0903 | 1 |
| Tyrosine | HGA | 0.11 | 0.15 | 0.1103 | 1 | 0.0399 | 1 |
| Tyrosine | HVA | −0.04 | −0.09 | 0.4513 | 1 | 0.2355 | 1 |
| Tyrosine | MHPG | −0.42 | −0.48 | 0 | 0 | 0 | 0 |
| Tyrosine | TYR | −0.03 | 0.08 | 0.5948 | 1 | 0.2033 | 1 |
| Tyrosine | VMA | −0.07 | 0.25 | 0.3867 | 1 | 0.001 | 0.055 |
| Tyrosine | HVA_by_TYR | −0.03 | −0.12 | 0.6612 | 1 | 0.1055 | 1 |
| Tyrosine | HGA_by_TYR | 0.13 | 0.13 | 0.0609 | 1 | 0.0709 | 1 |
| Tyrosine | 4 HPAC_by_TYR | 0.24 | 0.27 | 0 | 0.001 | 0 | 0 |
| Tyrosine | MHPG_by_TYR | −0.31 | −0.43 | 0 | 0.001 | 0 | 0 |
| Tyrosine | VMA_by_TYR | −0.06 | 0.23 | 0.4592 | 1 | 0.0035 | 0.187 |
| Phe/Try | 4HBAC | 0.14 | 0.3 | 0.0534 | 1 | 0.0001 | 0.007 |
| Other | AMTRP | −0.09 | 0.05 | 0.215 | 1 | 0.4271 | 1 |
| Other | 13PA | 0.07 | 0.13 | 0.206 | 1 | 0.0194 | 1 |
| Other | SA | 0.05 | 0.15 | 0.3594 | 1 | 0.0171 | 0.921 |
| Other | THEOPHYL1NE | −0.08 | 0.05 | 0.1805 | 1 | 0.3335 | 1 |

TABLE 21
Descriptive statistics and linear mixed model results on the 31 metabolites*

| | | | | Fold Change | | | Linear mixed model results - Week 4 | | | Linear mixed model results -Week 8 | |
|---|---|---|---|---|---|---|---|---|---|---|---|
| | Mean (SD) | | | BL_W | BL_W8 | W4 Coef Est | | W4 | W8 Coef Est | | W8 |
| Metabolites | BL | W4 | W8 | 4 FC | FC | (Conf. Int) | W4 p | Adjusted-p | (Conf. Int) | W8 p | Adjusted-p |
| 3OHKY | 0.02 | −0.1 | 0.1 | −0.12 | 0.08 | −0.1 | 0.049734 | 1 | 0.1 | 0.131769 | 1 |
| | (0.94) | (1.14) | (0.91) | | | (−0.2, −0.0) | 38 | | (−0.0, 0.2) | | |
| 4HBAC | −0.13 (1) | 0(0.91) | 0.16 | 0.14 | 0.3 | 0.1 | 0.053400 | 1 | 0.3 | 0.000132 | 0.12803 |
| | | | (0.98) | | | (−0.0, 0.3) | 39 | | (0.1, 0.4) | 4 | |
| 4HPAC | −0.18 | 0.04 | 0.13 | 0.22 | 0.31 | 0.2 | 0.000101 | 0.0979 | 0.3 | 8.71E-08 | 8.42E-05 |
| | (0.96) | (0.99) | (0.99) | | | (0.1, 0.3) | 28 | 38 | (0.2, 0.4) | | |
| 4HPLA | −0.03 | −0.06 | 0.05 | −0.03 | 0.07 | | 0.462213 | 1 | 0.1 | 0.09026237 | 1 |
| | (0.98) | (0.99) | (0.99) | | | | 652 | | (−0.0, 0.2) | 37 | |
| 5HIAA | 0.04 | −0.06 | 0.07 | −0.1 | 0.03 | −0.1 | 0.116414 | 1 | 0.0 | 0.572509 | 1 |
| | (1.06) | (0.98) | (0.98) | | | (−0.2, 0.0) | 685 | | (−0.1, 0.2) | 07 | |
| 5HT | 0.82 | −0.42 | −0.46 | −1.24 | −1.28 | −1.2 | 1.02E-62 | 9.88E-60 | −1.3 | 1.67E-64 | 1.62E-61 |
| | (0.97) | (0.72) | (0.72) | | | (−1.3, −1.1) | | | (−1.4, −1.2) | | |
| 5HTP | 0.09 | 0.04 | -0.09 | −0.05 | −0.18 | −0.0 | 0.510903 | 1 | −0.2 | 0.022860 | 1 |
| | (0.98) | (1.02) | (0.98) | | | (−0.2, 0.1) | 9 | | (−0.3, −0.0) | 17 | |
| AMTRP | 0.03 | −0.06 | 0.09 | −0.09 | 0.05 | −0.1 | 0.215047 | 1 | 0.1 | 0.427112 | 1 |
| | (1.02) | (0.99) | (0.95) | | | (−0.2, 0.1) | 877 | | (−0.1, 0.2) | 71 | |
| ATOCO | −0.06 | 0.02 | 0.08 | 0.08 | 0.14 | 0.1 | 0.329621 | 1 | 0.1 | 0.061744 | 1 |
| | (1.06) | (1.03) | (0.92) | | | (−0.1, 0.2) | 719 | | (−0.0, 0.3) | 3 | |
| CYS | 0.07 | −0.08 | 0.03 | −0.15 | −0.05 | −0.2 | 0.048802 | | −0.0 | 0.495902 | 1 |
| | (0.75) | (1.08) | (0.96) | | | (−0.3, −0.0) | 522 | | (−0.2, 0.1) | 43 | |
| DTOCO | −0.01 | −0.04 | 0.07 | −0.02 | 0.09 | −0.0 | 0.755037 | 1 | 0.1 | 0.221198 | 1 |
| | (1.03) | (1.02) | (0.92) | | | (−0.2, 0.1) | 825 | | (−0.1, 0.2) | 62 | |
| GTOCO.A | −0.07 | −0.02 | 0.1 | 0.05 | 0.17 | 0.1 | 0.523045 | 1 | 0.2 | 0.024599 | 1 |
| | (1.05) | (1.05) | (0.9) | | | (−0.1, .02) | 184 | | (0.0, 0.3) | 7 | |
| HGA | −0.08 | 0.02 | 0.06 | 0.11 | 0.15 | 0.1 | 0.110269 | 1 | 0.1 | 0.039859 | 1 |
| | (1.05) | (0.94) | (1.06) | | | (−0.0, 0.2) | 85 | | (0.0, 0.3) | 25 | |
| HVA | 0.06 | 0.02 | −0.03 | −0.04 | −0.09 | −0.0 | 0.451285 | 1 | −0.1 | 0.235450 | 1 |
| | (0.76) | (0.97) | (1.21) | | | (−0.2, 0.1) | 376 | | (−0.2, 0.1) | 53 | |
| HX | 0.15 | −0.04 | −0.1 | −0.19 | −0.25 | −0 2 | 0.010649 | 1 | −0.2 | 8.12E-05 | 0.07848 |
| | (0.85) | (1.21) | (0.95) | | | (−0.3, −0.0) | 287 | | (−0.4, −0.1) | | |
| I3AA | −0.1 | 0.04 | 0.08 | 0.14 | 0.18 | 0.1 | 0.010750 | 1 | 0.2 | 0.000437 | 0.42323 |
| | (1.01) | (1.03) | (0.94) | | | (0.0, 0.2) | 683 | | (0 1, 0.3) | 68 | |
| I3PA | −0.04 | 0.03 | 0.09 | 0.07 | 0.13 | 0.1 | 0.205957 | 1 | 0.1 | 0.019372 | 1 |
| | (0.97) | (0.96) | (0.97) | | | (−0.0, 0.2) | 62 | | (0.0, 0.2) | 34 | |
| KYN | 0.01 | −0.04 | 0.06 | −0.05 | 0.05 | −0.0 | 0.331986 | 1 | 0.1 | 0.425307 | 1 |
| | (0.89) | (0.92) | (1.16) | | | (−0.1, 0.0) | 513 | | (−0.1, 0.2) | 16 | |
| MET | 0.04 | −0.07 | 0.05 | −0.1 | 0.02 | −0.1 | 0.192244 | 1 | 0.0 | 0.799468 | |
| | (0.97) | (1) | (1.05) | | | (−0.3, 0.1) | 839 | | (−0.1, 0.2) | 45 | |
| MHPG | 0.28 | −0.14 | −0.19 | −0.42 | −0.48 | −0.4 | 652E-09 | 6.31E-06 | −0.5 | 3.53E-13 | 3.41E-10 |
| | (0.98) | (1.02) | (0.96) | | | (−0.6, −0.3) | | | (−0.6, −0.4) | | |
| SA | −0.07 (1) | −0.02 | 0.08 | 0.05 | 0.15 | 0.1 | 0.359371 | 1 | 0.1 | 0.017062 | 1 |
| | | (0.92) | (1.08) | | | (−0.1, 0.2) | 952 | | (0.0, 0.3) | 88 | |
| THEO- | 0.02 | −0.05 | 0.08 | −0.08 | 0.05 | −0.1 | 0.180453 | 1 | 0.1 | 0.333513 | 1 |
| PHYLINE | (0.88) | (1.09) | (0.94) | | | (−0.2, 0.0) | 024 | | (−0.1, 0.2) | 76 | |
| TRP | 0 (1.02) | −0.07 | 0.07 | −0.07 | 0.07 | −0.0 | 0.311358 | 1 | 0.1 | 0.315477 | 1 |
| | (1.05) | (0.98) | (1.02) | | | (−0.2, 0.0) | 058 | | (−0.0, 0.2) | 11 | |
| TYR | −0.02 | −0.05 | 0.06 | −0.03 | 0.08 | −0.0 | 0.594750 | | 0.1 | 0.203305 | |
| | (1.05) | (0.97) | (1) | | | (−0.2, 0.1) | 892 | | (−0.0, 0.2) | 49 | |
| URIC | −0.01 | −0.02 | 0.01 | −0.01 | 0.02 | −0.0 | 0.775885 | 1 | 0.0 | 0.653971 | 1 |
| | (0.95) | (1.05) | (1) | | | (−0.1, 0.1) | 084 | | (−0.1, 0.1) | 72 | |
| VMA | −0.07 | −0.14 | 0.18 | −0.07 | 0.25 | −0.1 | 0.386675 | 1 | 0.2 | 0.001015 | 0.98171 |
| | (1.01) | (1.12) | (0.86) | | | (−0.2, 0.1) | 434 | | (0.1, 0.4) | 21 | |
| XAN | 0.1 | 0.06 | −0.15 | −0.04 | −0.24 | −0.0 | 0.606808 | 1 | −0.2 | 0.001641 | 1 |
| | (0.97) | (1.06) | (0.98) | | | (−0.2, 0.1) | 352 | | (−0.4, −0.1) | 88 | |
| XANTH | −0.09 | −0.01 | 0.13 | 0.08 | 0.21 | 0.1 | 0.407704 | 1 | 0.2 | 0.008752 | 1 |
| | (1.29) | (0.95) | (0.61) | | | (−0.1, 0.3) | 025 | | (0.1, 0.4) | 23 | |
| G | 0.05 | 0.04 | −0.1 | −0.01 | −0.14 | −0.0 | 0.889392 | 1 | −0.1 | 0.049794 | 1 |
| | (0.99) | (0.98) | (1.04) | | | (−0.1, 0.1) | 941 | | (−0.3, -0.0) | 12 | |
| GR | 0.07 | −0.04 | −0.04 | −0.12 | −0.11 | −0.1 | 0.141293 | 1 | −0.1 | 0.1711391 | 1 |
| | (1.3) | (0.79) | (0.84) | | | (−0.3, 0.0) | 016 | | (−0.3, 0.0) | 7 | |
| PXAN | −0.05 | −0.03 | 0.1 | 0.03 | 0.15 | 0.0 | 0.619938 | 1 | 0.2 | 0.003392 | 1 |
| | (0.99) | (1.06) | (088) | | | (−0.1, 0.1) | 499 | | (0.1, 0.3) | 43 | |

*All data are in the logarithmic space, standardized to unit variance.

### Tryptophan Pathway

Dramatic changes were observed in serotonin (5HT) and the ratio 5HIAA/5HT, both at week 4 and week 8. At both time points, 5HT showed substantial decreases and the 5HIAA/5HT ratio was significantly elevated. While TRP itself did not show a notable change, its indole-containing metabolite I3AA was significantly elevated, as was the ratio of I3AA/TRP, possibly indicating a shift away from the serotonergic pathway of TRP metabolism. Interestingly, another indole-containing compound that is known to be produced only by gut microbiota in humans, I3PA, was also increased at 8 weeks (unadjusted p-value <0.02). No statistically significant alterations were observed in the KYN branch of TRP metabolism.

### Tyrosine Pathway

A similar trend of a shift to noncanonical branches of tyrosine metabolism was also observed in this pathway. MHPG, the major metabolite of the neurotransmitter norepinephrine and the ratio MHPG/TYR showed significant reductions in their blood levels at both 4 and 8 weeks while VMA, a norepinephrine end metabolite, showed significant elevations at 8 weeks compared with baseline. A phenolic acid, 4HPAC, and its ratio to TYR (4HPAC/TYR) were significantly increased at both 4 and 8 weeks. Another phenolic derivative from the phenylalanine/tyrosine pathway, 4-hydroxybenzoic acid (4HBAC), was also significantly elevated at 8 weeks.

### Purine Pathway

The purine metabolites HX and XAN and the ratio XAN/XANTH were decreased significantly, while the ratios PXAN/XAN and URIC/XAN were elevated at 8 weeks compared with baseline, indicating a similar decline in the canonical pathway of purine metabolism, as observed in the tryptophan and tyrosine pathways.

Other metabolites that showed significant changes, albeit at unadjusted p values <0.05, were the purine metabolites, G, PXAN, and XANTH; the TRP metabolite, 5HTP; the tyrosine metabolite, HGA; and other metabolites, such as salicylic acid (SA).

Metabolomic Changes Associated with Changes in Depressive Symptoms (HRSD₁₇)

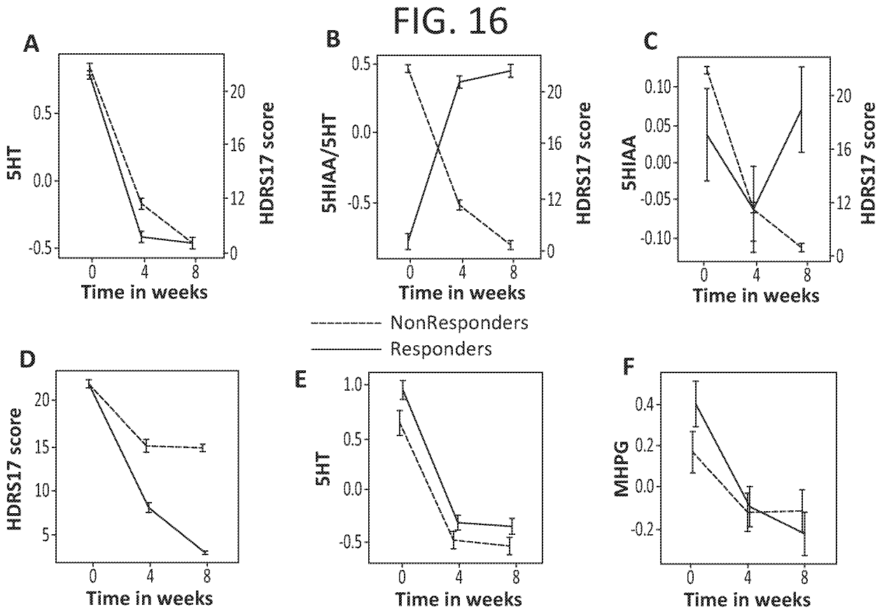

Using linear mixed models, we examined the association between temporal changes in metabolite levels (across three time points, baseline, 4 weeks, and 8 weeks) and the temporal changes in patients' HRSD₁₇ scores over that period of time (see FIG. 16A-C and Table 22). In the overall population, metabolites from the TRP pathway were associated with changes in HRSD₁₇ scores. 5HT, 5HIAA and the serotonin turnover marker 5HIAA/5HT showed significant positive and negative associations, respectively, with decreases over time in HRSD₁₇ scores (FDR-adjusted p values <0.01).

TABLE 22
Metabolites significantly associated with changes in HRSD₁₇ score over time.

| Pathway | Metabolites | Coef Estimate (95% CI) | | P-value | Adjusted P-value |
|---|---|---|---|---|---|
| Purine | GR/XANTH | 0.457 | (0.043, 0.871) | 0.03 | 1 |
| Tryptophan | 3OHKY | −0.560 | (−0.981, −0.139) | 0.009 | 0.431 |
| Tryptophan | 5HIAA | −0.819 | (−1.268, −0.370) | <0.001 | 0.017 |
| Tryptophan | 5HT | 1.724 | (1.246, 2.202) | <0.001 | <0.001 |
| Tryptophan | 5HTP | 0.442 | (0.020, 0.864) | 0.04 | 1 |
| Tryptophan | 5HTP/TRP | 0.452 | (0.027, 0.876) | 0.037 | 1 |
| Tryptophan | 5HIAA/TRP | −0.733 | (−1.198, −0.269) | 0.002 | 0.095 |
| Tryptophan | 5HIAA/5HTP | −0.567 | (−0.986, −0.148) | 0.008 | 0.379 |
| Tryptophan | 5HIAA/5HT | −1.998 | (−2.486. −1.511) | <0.001 | <0.001 |
| Tyrosine | HGA | −0.519 | (−0.943, −0.094) | 0.017 | 0.792 |
| Tyrosine | VMA | −0.491 | (−0.906, −0.077) | 0.02 | 0.954 |
| Tyrosine | HGA/TYR | −0.526 | (−0.952, −0.099) | 0.016 | 0.742 |

We further subcategorized the population based on their HRSD₁₇ scores after 8 weeks of treatment. If they had at least a 50% reduction in their HRSD₁₇ scores, from baseline to exit, they were categorized as responders, otherwise they were nonresponders. We examined whether the temporal associations between metabolite changes and HRSD₁₇ scores significantly differed between responders and nonresponders. The mean (±sd) HRSD₁₇ scores in the responders and nonresponders were 21.86 (±5.17) and 22.03 (±4.28), respectively, at baseline, 10.10 (±5.77) and 15.03 (±6.58), respectively, at week 4, and 5.79 (±3.27) and 14.90 (±4.15), respectively, at week 8. 5HT temporal profiles significantly differed between the two groups, with the levels being consistently higher in the responders at baseline, week 4, and week 8, while the decline in HRSD₁₇ scores was significantly lower at both 4 and 8 weeks compared with baseline (FIG. 16D-E). Levels of MHPG at baseline were significantly higher and the drop in MHPG levels over time was significantly greater in responders compared with nonresponders (FIG. 16F).

Relationships Amongst Metabolites at Baseline and after 8 Weeks of Treatment

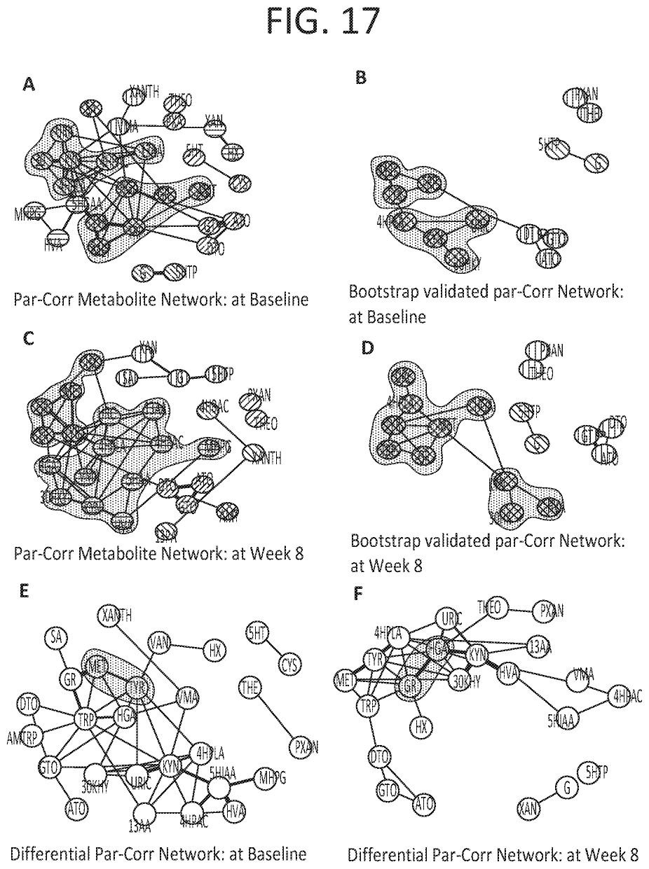

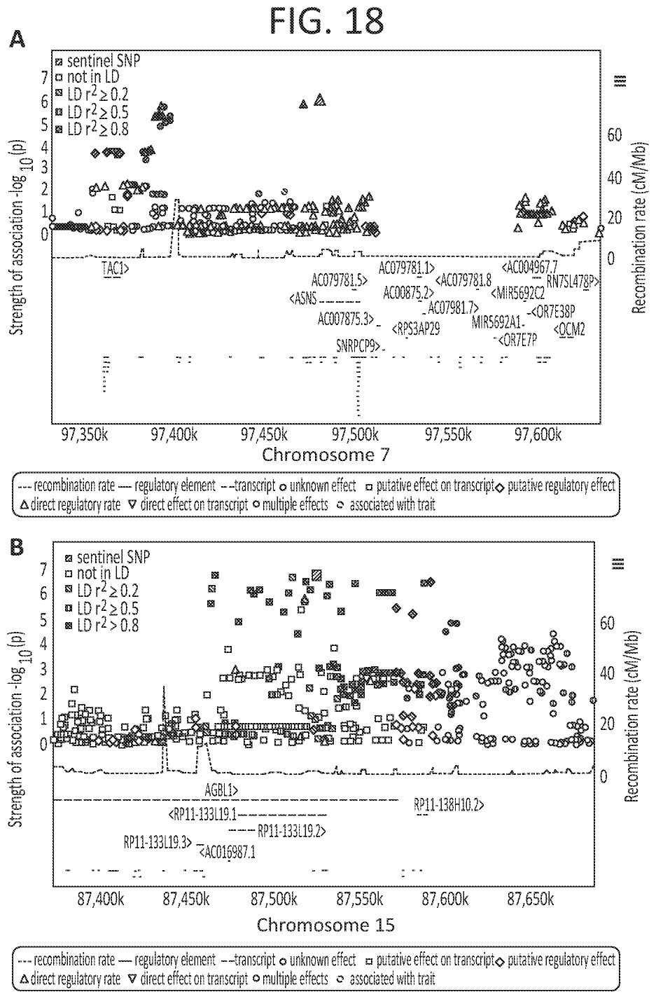

Biological systems are now increasingly viewed as complex networks of interlinked entities, topological analyses of which can reveal the underlying landscape of biological functionalities. Gaussian graphical modeling has been used to reconstruct pathway reactions in metabolomics data (Krumsiek et al., BMC Syst. Biol. 5, 21 (2011)). Combining a partial correlation network and genetic variation through GWAS has been shown to provide an in-depth overview of the underlying mechanistic pathways (Shin et al., Nat. Genet. 46, 543 (2014)). Here, using regularized partial correlation network analysis at baseline and also after week 8 of drug exposure (FIG. 17), we assessed the metabolite-metabolite interactions between tryptophan, tyrosine, purine, and tocopherol pathways.

Regularized partial correlation networks of the metabolites at baseline (FIG. 17A-B) and also at week 8 (FIG. 17C-D) showed significant correlations between several metabolites, both within and between pathways forming clusters of interacting molecules. A list of statistically significant partial correlations between metabolites at baseline and week 8 are presented in Tables 23-24. Important observations through duster subgraph analysis showed that MET, TYR, and TRP formed a tight cluster both at baseline and week 8. However, the strength of interactions between MET and TYR was significantly reduced at week 8, compared with baseline (˜50% reduction, permutation p value <0.10). GR connection to this duster was significant at week 8 through interactions with all three metabolites. HVA formed a significant correlation with KYN at week 8 that was not observed at baseline. Multiple other overlapping correlations in the two networks were observed at both baseline and week 8, suggesting that the majority of these interactions were a result of housekeeping biological interactions and were probably not entirely related to the drug effect.

TABLE 23
Significant Partial Correlations between Metabolites at Baseline

| pcor | node1 | node2 | pval | gval | prob |
|---|---|---|---|---|---|
| 0.675121 | PXAN | THEO- | 2.22E−16 | 3.42E−14 | 1 |
| | | PHYLINE | | | |
| 0.497369 | DTOCO | GTOCO.A | 2.22E−16 | 3.42E−14 | 1 |
| 0.441644 | ATOCO | GTOCO.A | 2.22E−16 | 3.42E−14 | 1 |
| 0.356224 | 3OEHKY | KYN | 7.74E−11 | 8.92E−09 | 1 |
| 0.350343 | G | 5HTP | 1.65E−10 | 1.51E−08 | 1 |
| 0.347158 | MET | TYR | 2.47E−10 | 1.90E−08 | 0.999733 |
| 0.272889 | 4HPLA | TYR | 8.98E−07 | 5.49E−05 | 0.999733 |
| 0.270203 | TRP | TYR | 1.16E−06 | 6.68E−05 | 0.999633 |
| 0.264603 | ATOCO | DTOCO | 1.95E−06 | 0.0001 | 0.998614 |
| 0.254313 | HX | XAN | 4.96E−06 | 0.000229 | 0.992514 |
| 0.237321 | MET | TRP | 2.12E−05 | 0.000889 | 0.959223 |
| 0.206711 | GR | TRP | 0.000224 | 0.007769 | 0.959223 |
| 0.206346 | 5HIAA | HVA | 0.00023 | 0.007935 | 0.959223 |
| 0.203251 | I3AA | 4HPAC | 0.000286 | 0.009436 | 0.90876 |
| 0.192854 | XANTH | VMA | 0.000586 | 0.017419 | 0.90876 |
| 0.187816 | TRP | AMTRP | 0.000818 | 0.022612 | 0.90876 |
| 0.186702 | KYN | 4HPLA | 0.000879 | 0.023872 | 0.816552 |
| 0.177562 | GR | 4HBAC | 0.001573 | 0.038717 | 0.816552 |

TABLE 24
Significant Partial Correlations between Metabolites at Week 8

| pcor | node1 | node2 | pval | gval | prob |
|---|---|---|---|---|---|
| 0.658993 | PXAN | THEOPHYLINE | 2.22E−16 | 5.14E−14 | 1 |
| 0.464725 | DTOCO | GTOCO.A | 2.22E−16 | 5.14E−14 | 1 |
| 0.40203 | ATOCO | GTOCO.A | 4.97E−14 | 7.68E−12 | 0.999999 |
| 0.320313 | G | 5HTP | 3.58E−09 | 3.14E−07 | 0.999999 |
| 0.319619 | TRP | TYR | 3.89E−09 | 3.29E−07 | 0.999999 |
| 0.317972 | ATOCO | DTOCO | 4.71E−09 | 3.64E−07 | 0.999903 |
| 0.282116 | KYN | HVA | 2.39E−07 | 1.55E−05 | 0.999903 |
| 0.276417 | 3OHKY | KYN | 4.24E−07 | 2.46E−05 | 0.998525 |
| 0.245298 | G | XAN | 7.86E−06 | 0.000352 | 0.998525 |
| 0.244773 | 4HPLA | TYR | 8.23E−06 | 0.000365 | 0.998525 |
| 0.242564 | URIC | 4HPLA | 9.98E−06 | 0.00042 | 0.995347 |
| 0.234402 | GR | TRP | 2.00E−05 | 0.000773 | 0.974866 |
| 0.218193 | TRP | AMTRP | 7.43E−05 | 0.002647 | 0.925041 |
| 0.191281 | DTOCO | TRP | 0.000533 | 0.015595 | 0.925041 |
| 0.190841 | MET | TYR | 0.000549 | 0.01597 | 0.925041 |
| 0.185741 | URIC | HGA | 0.000775 | 0.020724 | 0.925041 |
| 0.183455 | MET | GR | 0.000902 | 0.023072 | 0.925041 |
| 0.181866 | KYN | 4HPLA | 0.001002 | 0.02477 | 0.925041 |
| 0.180506 | G | AMTRP | 0.001094 | 0.026261 | 0.925041 |
| 0.178186 | XANTH | I3PA | 0.001271 | 0.028869 | 0.925041 |
| 0.177028 | HX | XAN | 0.001369 | 0.030194 | 0.589758 |

Differential Partial Correlation Networks Associated with HRSD₁₇ Scores at Week 8

HRSD₁₇ scores at week 8 indicated the depression status of the patients post drug treatment. We compared two partial correlation networks constructed with lower and higher values of HRSD₁₇ scores at week 8 (the outcome status), using a median split, as a node. Our aim was to examine if the associations between metabolites were different between patients who responded to the drug better than those who responded poorly. Several metabolite-metabolite associations across the tyrosine, tryptophan, and purine pathways were found to be changed as a function of higher or lower outcome status. At baseline, GR-MET, TYR-MET, and KYN-URIC partial correlations were most impacted, while at week 8, KYN-HVA, KYN-3OHKY, 5HTP-G, and HGA-GR values were most impacted by HRSD₁₇ week 8 status (FIG. 17E, 17F). Two sets of metabolite-metabolite interactions associated with the outcome status, HGA-GR interactions at week 8 and MET-TYR interactions at baseline, were further found to be statistically significant in linear regression models (highlighted in yellow in FIG. 17E, 17F). An interesting observation from the differential analysis of networks at baseline was that the partial correlations between metabolites that were differential between the low versus high HRSD₁₇ networks involved several gut-microbe-related metabolites such as HGA, I3AA, 5HIAA, 4HPLA, and 4HPAC amongst others (FIG. 17E).

### Genetic Influences on Ratios of Interacting Metabolite Pairs Change During SSRI Treatment

To identify potential modulators of significant metabolite-metabolite interactions and their differential interactions over time, we performed genome-wide association studies with the pairwise ratios of HGA/GR and MET/TYR in 288 subjects at each time point. To this end, we computed additive genetic associations of the two ratios with 5.55 mio autosomal SNPs at each time point, while adjusting for age, sex, time point-specific HRSD₁₇ score, the first five PCs to account for population stratification. The strongest signal for the HGA/GR ratio was for rs55933921 on chromosome 7 (baseline: P=8.59×10⁻⁷; week 4: P=3.05×10⁻³; week 8: P=1.14×10⁻³) in a locus spanning two genes, TAC1 (protachykinin-1) and ASNS (asparagine synthetase [glutamine-hydrolyzing]). The strongest signal for the MET/TYR ratio was for rs2701431 on chromosome 15 (baseline: P=5.57×10⁻³; week 4: P=2.00×10⁻⁴; week 8: P=8.48×10⁻⁸) in the AGBL1 (ATP/GTP-binding protein like 1) locus (FIG. 18A-B). Of note, genetic associations between these loci and metabolite ratios were the strongest at the time point that showed insignificant metabolite-metabolite interactions on the HRSD₁₇ score.

### Example 5

In this study 180 metabolites from the classes in Table 25 were assessed using FIA and UPLC platforms as described previously.

TABLE 25
Metabolites and Biological Relevance

| Metabolite Class | Biological Relevance |
|---|---|
| Acylcarnitines | Energy metabolism, fatty acid transport |
| | and mitochondrial fatty acid oxidation |
| | (e.g., inborn disorders such as MCAD), |
| | ketosis, oxidative stress, mitochondrial |
| | membrane damage (apoptosis) |
| Amino acids | Amino acid metabolism (e.g., inborn |
| | disorders such as PKU, MSUD), urea |
| | cycle, activity of gluconeogenesis and |
| | glycolysis, insulin sensitivity/resistance, |
| | neurotransmitter metabolism, oxidative |
| | stress |
| Biogenic amines | Neurological disorders, cell proliferation, |
| | cell cycle progression, DNA stability, |
| | oxidative stres |
| Hexoses | Carbohydrate metabolism |
| Phosphatidylcholines | Dyslipidemia, membrane composition |
| | and damage, fatty acid profile, activity |
| | of desaturases |
| Lyso Phosphatidylcholines | Degradation, of phospholipids (phospho- |
| | lipase activity), membrane damage, |
| | signaling cascades, fatty acid profile |
| Sphingomyelins | Signaling cascades, membrane damage |
| | (e.g., neurodegeneration) |

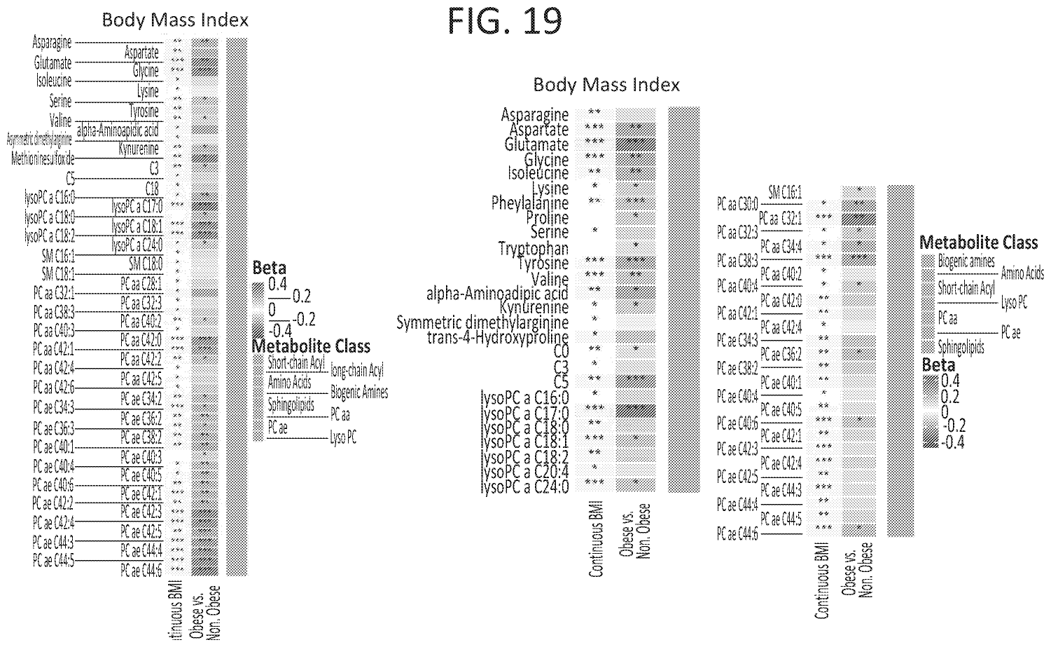

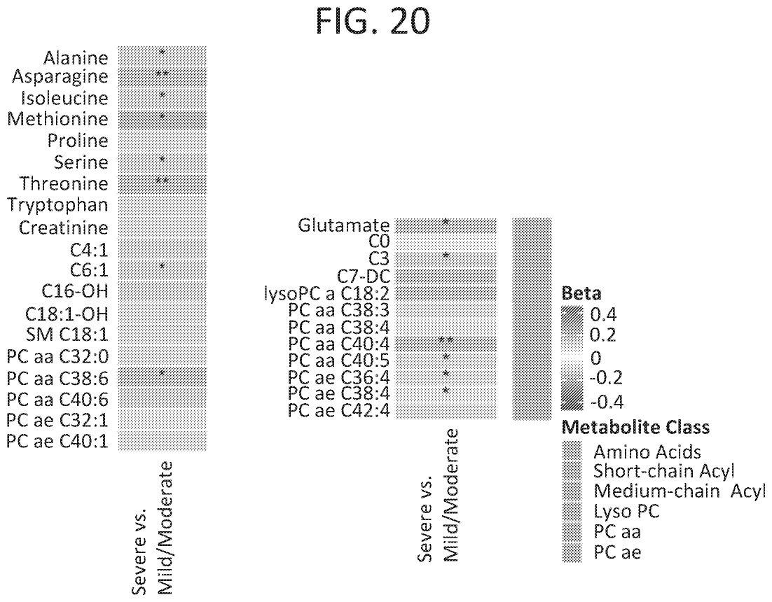

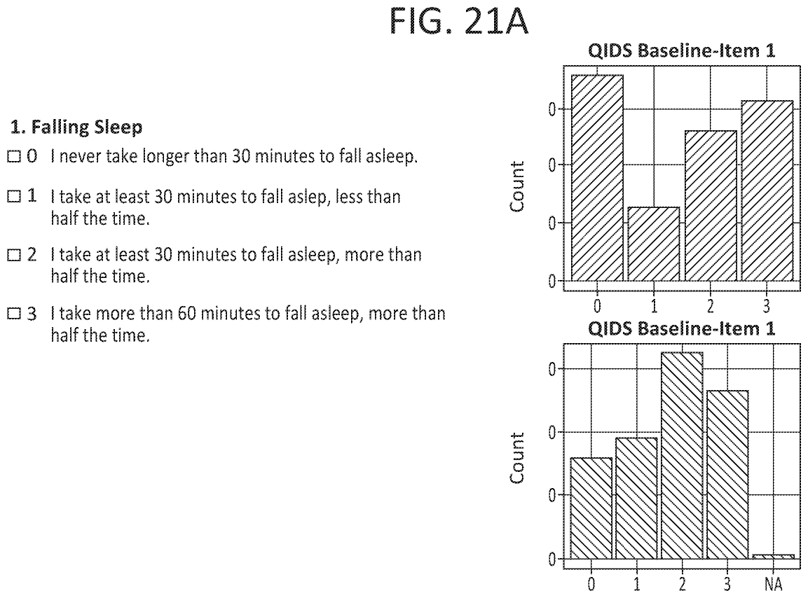

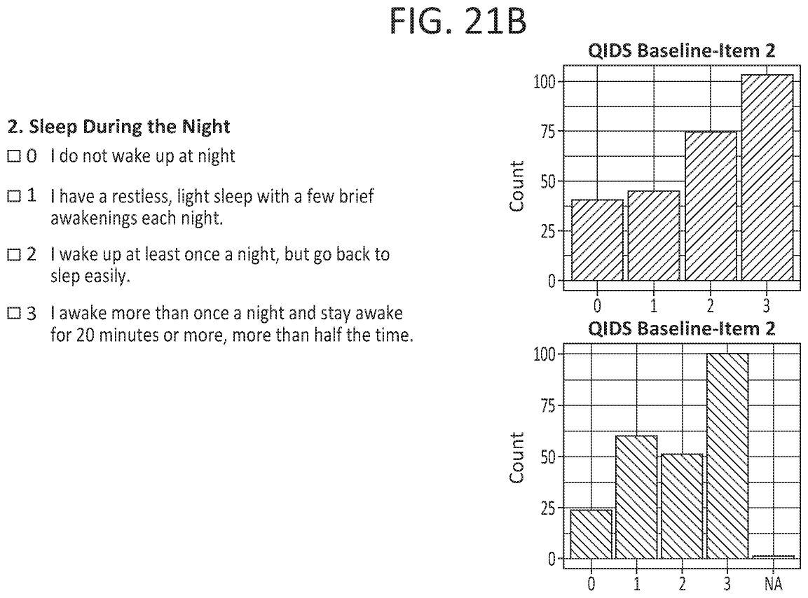

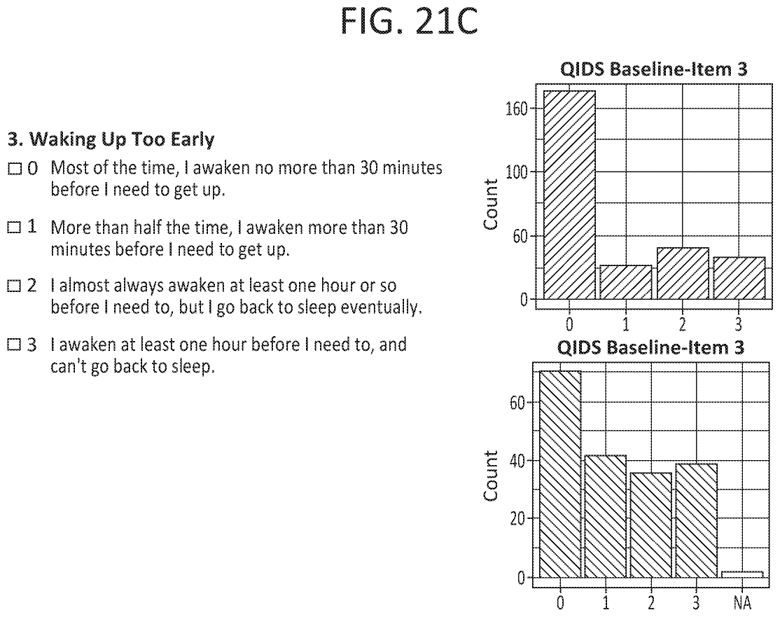

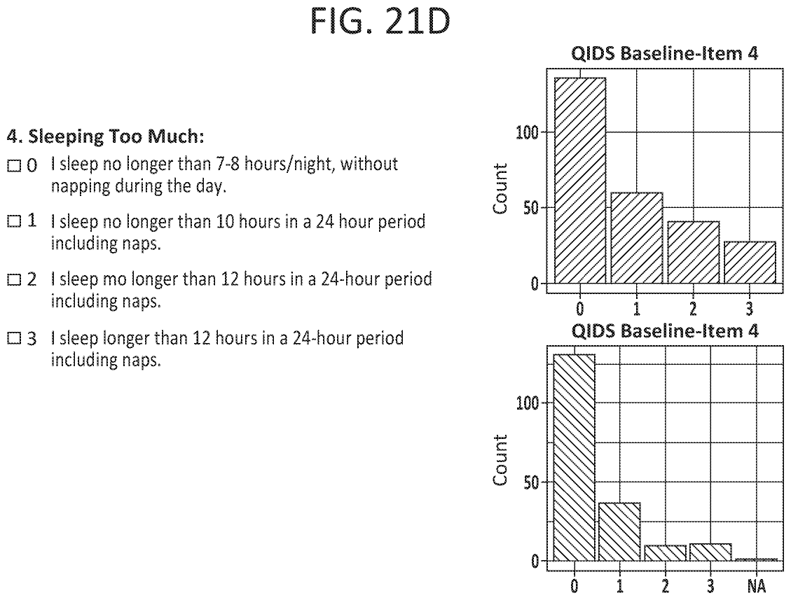

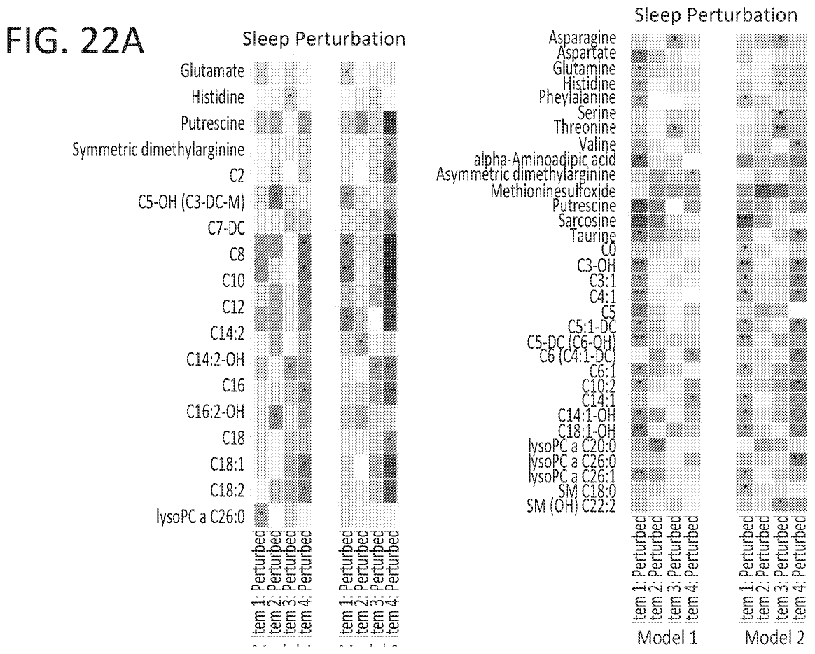

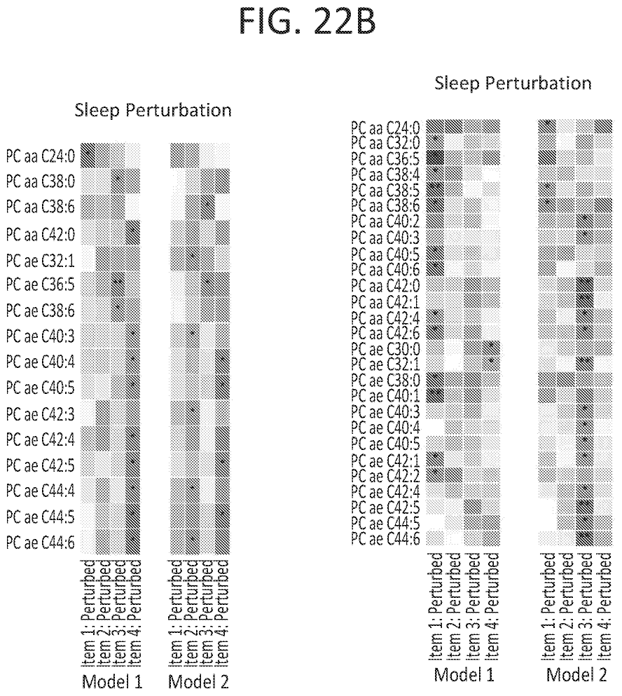

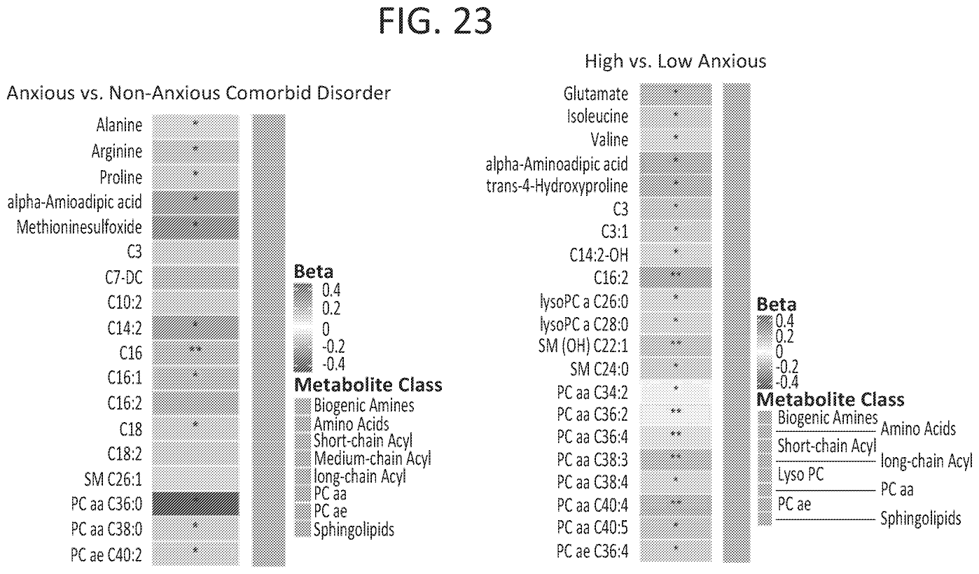

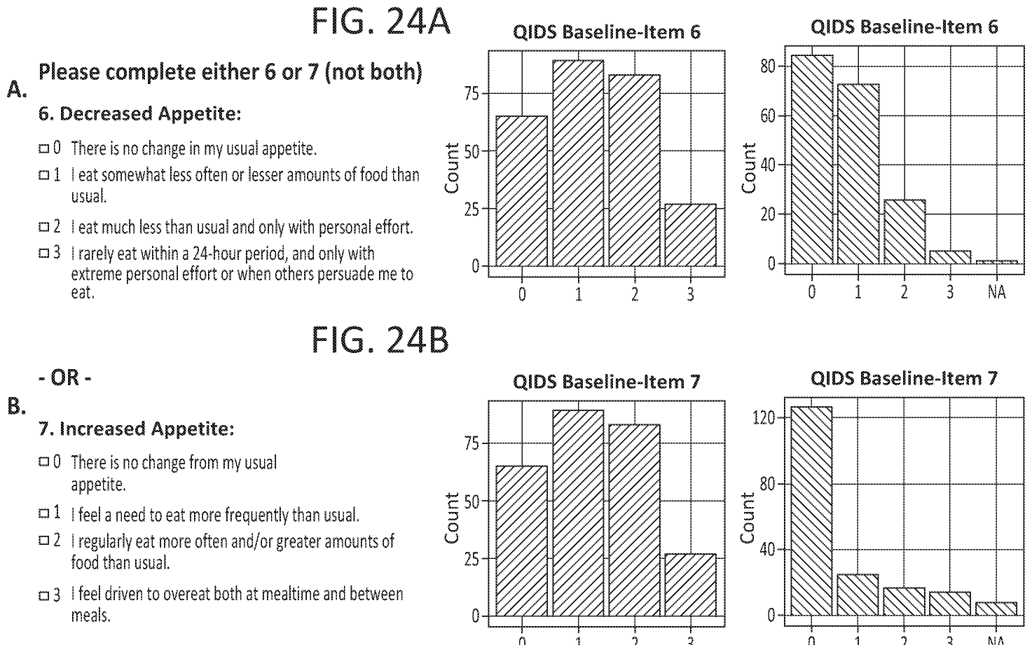

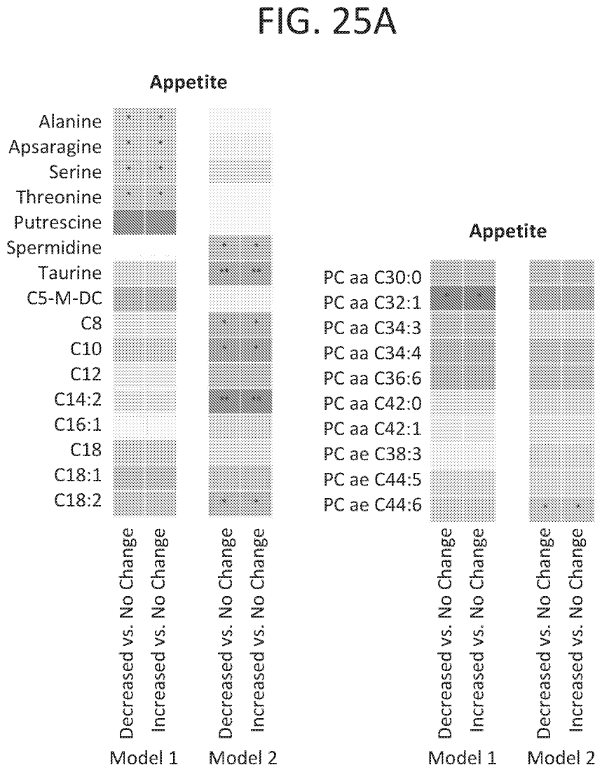

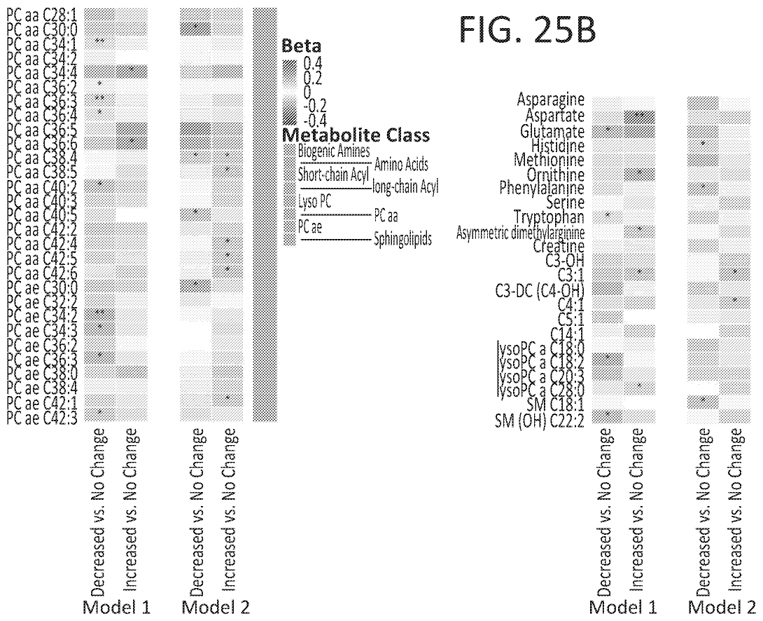

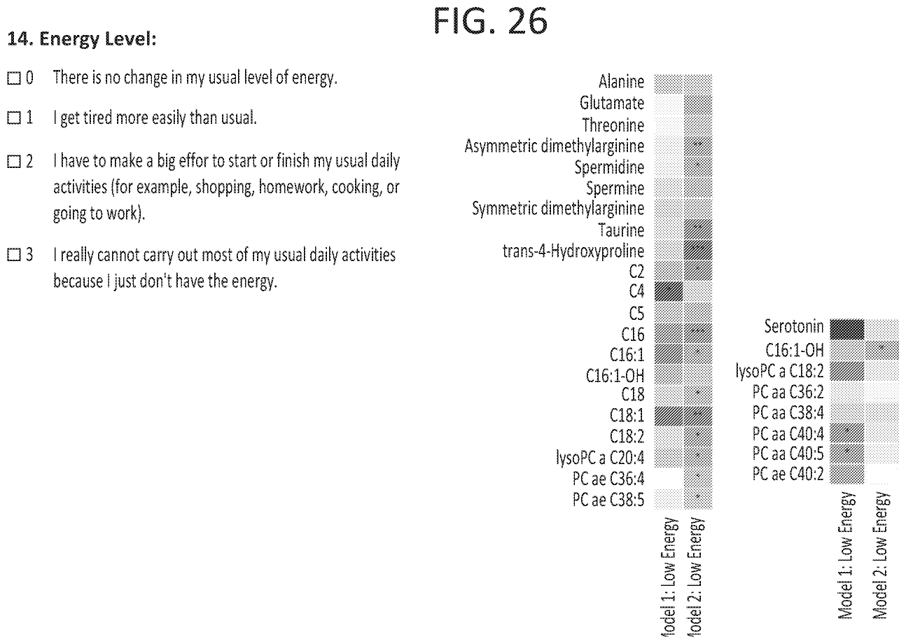

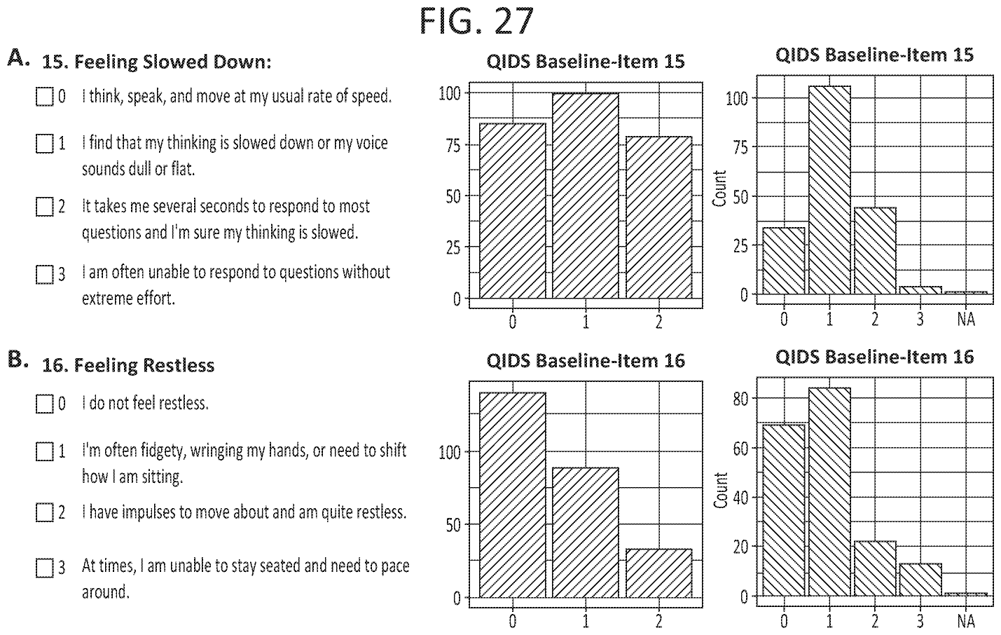

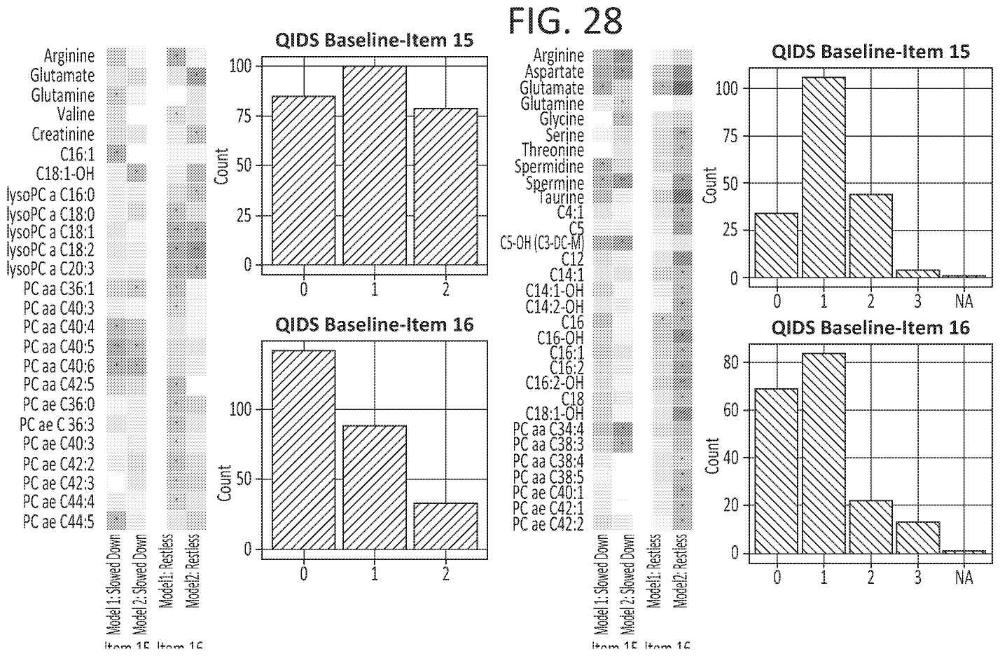

Baseline comparisons of metabolites were done for each of the following: (a) depression severity, (b) sleep perturbation, (c) anxiety—high anxious vs low anxious, (d) anxious vs non-anxious comorbid disorder, (e) appetite, (f) energy level, (g) psychomotor changes: slowed down and restless, and (h) body mass index. Results were adjusted for age and sex and are shown in FIGS. 19-28.

Antidepressant exposure resulted in change in metabolites related to gut microbiome including increase in hippuric acid, 3 indolepropionic acid, short-chain fatty acids including C3:0, C5:0, succinic acid, secondary bile acids including (tauroursodeoxycholic acid (TDCA) and glycoursodeoxycholic acid (GUDCA). In addition, after 12-weeks of treatment, changes in medium and long-chain fatty acids including (C6:0, C9:0, C13:0, C15:0, C16:0, C17:0, C18:0, C20:3 (cis-8,11,14), C22:4 (cis-7,10,13,16)) were associated with change in HRSD₁₇.

Some fatty acids including 3-hydroxybutyric acid, acetic acid, pelargonic acid, EPA (C20:5 (cis-5,8,11,14,17)), arachidonic acid (C20:4 (cis-5,8,11,14)), C22:4 (cis-7,10,13,16), C22:5 (cis-7,10,13,16,19) were differential between severe and mild depressed patients.

Hard-to-fall-asleep patients had consistently increased fatty acids, especially, polyunsaturated fatty acids (PUFAs), but not saturated fatty acids (C10, C12, and C15), while in hard-to-sustain-sleep individuals (item 2) these fatty acids were all depleted.

Low anxious depression is associated with significantly lower levels of amino acids (L-lysine, L-serine, L-asparagine, glycine, leucine and 3-methyl-2-oxovaleric acid) and secondary bile acids (TDCA, GLCA-3S, GDCA, 7-ketoLCA, 12-ketoLCA, alloLCA, isoLCA), and significantly higher levels of fatty acids, both saturated and unsaturated fatty acids (citric acid, (±)-2-methylpentanoicacid, pelargonic acid, C11:0, C20:5 (cis-5,8,11,14,17), C14:0, C15:0, C22:6 (cis-4,7,10,13,16,19), C22:5 (cis-7,10,13,16,19), and C19:2 (cis-10,13)).

In low energy depressed individuals, unconjugated bile acids—CDCA, CA, sioLCA, and LCA were lower.

In the future studies, metabolite changes with be compared among the following groups: (a) medication-treated vs CBT-treated among all completers; (b) change for the QIDS-SR items and change in microbiome (including QIDS-SR_14, QIDS-SR_06 and QIDS-SR_07, QIDS-SR_01 and QIDS-SR_04); (c) change in HRSD₁₇ score and change in microbiome (including continuous change, remission vs non-remission and remitter vs. treatment-failure); and (d) change in BMI.

What is claimed:

1. A method for diagnosing or detecting depression in a subject, the method comprising:
obtaining a sample from a subject and determining concentration levels of one or more biomarker metabolites in the sample from the subject; and
diagnosing the subject as having depression or an increased risk of depression when the concentration levels of the one or more biomarker metabolites in the sample from the subject is different from (greater or less) than concentration levels the one or more biomarker metabolites in a control sample.

2. The method of claim 1, further comprising:
initially treating the subject for depression by administering an effective amount sufficient to attenuate, reduce, or eliminate the symptoms of depression of one or more of antidepressants, cognitive behavior therapy, exercise, dietary supplements, prebiotics, probiotics, dietary changes, or an elimination diet;
obtaining a second sample from the subject and determining concentration levels of one or more biomarker metabolites in the sample from the subject;
evaluating the concentration level of the one or more biomarker metabolites in comparison to control concentration levels of the one or more biomarker metabolites;
evaluating the efficacy of the depression treatment; and
continuing the one or more initial depression treatments; administering one or more additional depression treatment; or administering one or more second depression treatments (switching the treatment regimen).

3. The method of claim 1, wherein the biomarker metabolite comprises one or more of:
Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine);
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine);
Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine;
Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline;
Glycerophospholipids (i.e., PC aa): PC aa C24:0; PC aa C26:0; PC aa C28:1; PC aa C30:0; PC aa C32:0; PC aa C32:1; PC aa C32:3; PC aa C34:1; PC aa C34:2; PC as C34:3; PC as C34:4; PC aa C36:0; PC aa C36:1; PC as C36:2; PC as C36:3; PC aa C36:4; PC as C36:5; PC as C36:6; PC as C38:0; PC as C38:3; PC aa C38:4; PC aa C38:5; PC aa C38:6; PC aa C40:1; PC aa C40:2; PC aa C40:3; PC aa C40:4; PC aa C40:5; PC aa C40:6; PC aa C42:0; PC aa C42:1; PC aa C42:2; PC aa C42:4; PC aa C42:5; or PC aa C42:6;
Glycerophospholipids (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6;
Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1;
Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1;
short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs); secondary bile acids (tauroursodeoxycholic acid (TDCA) and glycoursodeoxycholic acid (GUDCA));
or combinations thereof.

4. The method of claim 1, wherein the biomarker metabolite comprises one or more of: tryptophan (Trp), tyrosine (Tyr), phenylalanine (Phe), methionine (Met), cysteine (Cys), 3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline; or combinations thereof.

5. The method of claim 1, wherein the biomarker metabolites comprise one or more of: carnitine, propionylcarnitine; butyrylcarnitine, isovalerylcarnitine, α-aminoadipic acid, glutamate and proline; isoleucine, valine, tryptophan, tyrosine, phenylalanine, methionine, methionine-sulfoxide, sarcosine; phosphatidylcholines, or sphingolipids.

6. The method of claim 1, wherein the biomarker metabolite concentration level is greater than the control concentration level.

7. The method of claim 1, wherein the biomarker metabolite concentration level is less than the control concentration level.

8. The method of claim 1, wherein two or more biomarker metabolite concentration levels covary and are greater than the control or covary and are less than the control concentration levels (positive correlation).

9. The method of claim 1, wherein two or more biomarker metabolite concentration levels vary dissimilarly (negative correlation) compared to the control concentration levels.

10. The method of claim 1, wherein the sample from the subject is one or more of whole blood, serum, plasma, urine, saliva, or other body fluids.

11. The method of claim 1, wherein the control sample is from an untreated subject or a subject or a population of subjects not experiencing depression or not at risk for depression.

12. The method of claim 1, wherein the depression is Major Depression Disorder (MDD), core depression (CD+), anxious depression (ANX+), neurovegetative symptoms of melancholia (NVSM+), or subclinical characteristics associated with depression.

13. The method of claim 2, wherein the antidepressant comprises tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

14. The method of claim 2, wherein the antidepressant is one or more selective serotonin reuptake inhibitors (antidepressant).

15. The method of claim 2, wherein the antidepressant comprises escitalopram, citalopram, fluoxetine, sertraline, paroxetine, fluvoxamine, vilazodone, vortioxetine, or duloxetine.

16. The method of claim 2, wherein the efficacy of the depression treatment is evaluated using the Hamilton Depression Rating Scale (HRSD₁₇), the Quick Inventory of Depressive Symptomatology (QIDS), subscales thereof, or specific questions thereof.

17. A method of treating depression comprising:
administering an effective amount sufficient to attenuate, reduce, or eliminate the symptoms of depression of an antidepressant or cognitive behaviorial therapy or a combination thereof to a subject having depression or at risk of depression;
obtaining a blood sample from the subject; and
measuring concentration levels in the subject's blood sample of one or more biomarker metabolites comprising: short-chain acylcarnitines, medium-chain acylcarnitines, long-chain acylcarnitines, amino acids, biogenic amines, glycerophospholipids, sphingolipids, or combinations thereof.

18. The method of claim 17, wherein the treatment is maintained or adjusted based on the concentration levels of one or more biomarker metabolites, ratios of biomarker metabolites, or combinations thereof.

19. The method of claim 17, wherein the depression is Major Depression Disorder (MDD), core depression (CD+), anxious depression (ANX+), neurovegetative symptoms of melancholia (NVSM+), or subclinical characteristics associated with depression.

20. The method of claim 17, wherein the antidepressant is one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

21. The method of claim 17, wherein the antidepressant comprises one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

22. The method of claim 17, wherein the antidepressant comprises escitalopram or citalopram.

23. The method of claim 17, wherein the treatment further comprises administering one or more of exercise, dietary supplements, prebiotics, probiotics, probiotics, dietary changes, or an elimination diet.

24. A method of treating depression and evaluating the treatment efficacy comprising:
identifying a subject suffering from or at risk of developing depression;
administering an effective amount sufficient to attenuate, reduce, or eliminate the symptoms of depression of one or more antidepressants, cognitive behavior therapy, exercise, dietary supplements, prebiotics, probiotics, or dietary changes, or a combination thereof over a period of time;
obtaining a sample from the subject and evaluating concentration levels of one or more biomarker metabolites;
evaluating the concentration level of the one or more biomarker metabolites in comparison to control concentration levels of the one or more biomarker metabolites;
evaluating the efficacy of the depression treatment; and
continuing the administration one or more first depression treatments; administering an additional depression treatment; or administering one or more second depression treatments (switching the treatment regimen).

25. The method of claim 24, wherein the biomarker metabolite comprises one or more of:
Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine);
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine);
Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine;
Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline;
Glycerophospholipids (i.e., PC aa): PC aa C24:0; PC aa C26:0; PC as C28:1; PC aa C30:0; PC aa C32:0; PC aa C32:1; PC aa C32:3; PC aa C34:1; PC aa C34:2; PC aa C34:3; PC aa C34:4; PC aa C36:0; PC aa C36:1; PC aa C36:2; PC aa C36:3; PC aa C36:4; PC aa C36:5; PC aa C36:6; PC aa C38:0; PC aa C38:3; PC aa C38:4; PC aa C38:5; PC aa C38:6; PC aa C40:1; PC aa C40:2; PC aa C40:3; PC as C40:4; PC as C40:5; PC aa C40:6; PC aa C42:0; PC as C42:1; PC as C42:2; PC aa C42:4; PC aa C42:5; or PC aa C42:6;
Glycerophospholipid ethers (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6;
Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1;
Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1;
short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cs-10,13); polyunsaturated fatty acids (PUFAs);
secondary bile acids (tauroursodeoxycholic acid (TDCA) and glycoursodeoxycholic acid (GUDCA));
3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline;
or combinations thereof.

26. The method of claim 24, wherein the sample from the subject is one or more of whole blood, serum, plasma, urine, saliva, or other body fluids.

27. The method of claim 24, wherein the control sample is from an untreated subject or a subject or population of subjects not experiencing depression or not at risk for depression.

28. The method of claim 24, wherein the biomarker metabolite concentration level is greater than the control concentration level.

29. The method of claim 24, wherein the biomarker metabolite concentration level is less than the control concentration level.

30. The method of claim 24, wherein two or more biomarker metabolite concentration levels covary and are greater than the control or covary and are less than the control concentration levels (positive correlation).

31. The method of claim 24, wherein two or more biomarker metabolite concentration levels vary dissimilarly (negative correlation) compared to the control concentration levels.

32. The method of claim 24, wherein the period of time is at least: 4-weeks, 8-weeks, 16-weeks, 32-weeks, 64-weeks, or greater than 64-weeks.

33. The method of claim 24, wherein the depression is Major Depression Disorder (MDD), core depression (CD+), anxious depression (ANX+), neurovegetative symptoms of melancholia (NVSM+), or subclinical characteristics associated with depression.

34. The method of claim 24, wherein the antidepressant is one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

35. The method of claim 24, wherein the antidepressant comprises of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

36. The method of claim 24, wherein the antidepressant comprises escitalopram or citalopram.

37. The method of claim 24, wherein the efficacy of the depression treatment is evaluated using the Hamilton Depression Rating Scale (HRSD₁₇), the Quick Inventory of Depressive Symptomatology (QIDS), subscales thereof, or specific questions thereof.

38. A method of evaluating the efficacy of depression treatment, the method comprising:
identifying a subject suffering from or at risk of developing depression;
obtaining a sample from the subject and determining concentration levels of one or more biomarker metabolites in the sample from the subject; and
diagnosing the subject as having depression or an increased risk of depression when the concentration levels of the one or more biomarker metabolites in the sample from the subject is different from (greater or less) than the concentration levels the one or more biomarker metabolites in a control sample.
administering an effective amount sufficient to attenuate, reduce, or eliminate the symptoms of depression of one or more first depression treatments over a period of time;
obtaining an additional sample from the subject and reevaluating the concentration level of the one or more biomarker metabolites in comparison to the subject's initial concentration levels of the one or more biomarker metabolites and the control concentration levels of the one or more biomarker metabolites;
evaluating the efficacy of the depression treatment; and
continuing the one or more first depression treatments, administering an additional depression treatment, or administering one or more second depression treatments (switching the treatment regimen).

39. The method of claim 38, wherein the biomarker metabolite comprises one or more of:
Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine);
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine);
Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine;
Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline;
Glycerophospholipids (i.e., PC aa): PC aa C24:0; PC aa C26:0; PC as C28:1; PC aa C30:0; PC aa C32:0; PC aa C32:1; PC aa C32:3; PC aa C34:1; PC aa C34:2; PC aa C34:3; PC aa C34:4; PC aa C36:0; PC aa C36:1; PC aa C36:2; PC aa C36:3; PC aa C36:4; PC aa C36:5; PC aa C36:6; PC aa C38:0; PC aa C38:3; PC aa C38:4; PC aa C38:5; PC aa C38:6; PC aa C40:1; PC aa C40:2; PC aa C40:3; PC as C40:4; PC as C40:5; PC aa C40:6; PC aa C42:0; PC as C42:1; PC as C42:2; PC aa C42:4; PC aa C42:5; or PC aa C42:6;
Glycerophospholipids (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6;
Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1;
Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1;
short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cs-10,13); polyunsaturated fatty acids (PUFAs);
secondary bile acids (tauroursodeoxycholic acid (TDCA) and glycoursodeoxycholic acid (GUDCA));
3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline;
or combinations thereof.

40. The method of claim 38, wherein the sample from the subject is one or more of whole blood, serum, plasma, urine, saliva, or other body fluids.

41. The method of claim 38, wherein the control sample is from an untreated subject or a population of subjects not experiencing depression or not at risk for depression.

42. The method of claim 38, wherein the biomarker metabolite concentration level increases compared to the initial sample or control.

43. The method of claim 38, wherein the biomarker metabolite concentration level decreases compared to the initial sample or control.

44. The method of claim 38, wherein two or more biomarker metabolite concentration levels covary and increase or covary and decrease (positive correlation) compared to the initial sample or control.

45. The method of claim 38, wherein two or more biomarker metabolite concentration levels vary dissimilarly (negative correlation) the initial sample or control.

46. The method of claim 38, wherein the efficacy of the depression treatment is evaluated using the Hamilton Depression Rating Scale (HRSD₁₇), the Quick Inventory of Depressive Symptomatology (QIDS), subscales thereof, or specific questions thereof.

47. The method of claim 38, wherein the period of time is at least: 4-weeks, 8-weeks, 12-weeks, 16-weeks, 32-weeks, 64-weeks, or greater than 64-weeks.

48. The method of claim 38, wherein the depression is Major Depression Disorder (MDD), core depression (CD+), anxious depression (ANX+), neurovegetative symptoms of melancholia (NVSM+), or subclinical characteristics associated with depression.

49. The method of claim 38, wherein the first depression treatments comprises one or more of antidepressants, cognitive behavior therapy, exercise, dietary supplements, prebiotics, probiotics, dietary changes, or an elimination diet.

50. The method of claim 49, wherein the antidepressant is one or more of tranylcypromine, pheneizine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

51. The method of claim 49, wherein the antidepressant comprises escitalopram, citalopram, fluoxetine, sertraline, paroxetine, fluvoxamine, vilazodone, vortioxetine, or duloxetine.

52. The method of claim 49, wherein the antidepressant comprises escitalopram or citalopram.

53. A method for screening a subject for depression or an increased risk of depression, the method comprising:
obtaining a sample from the subject and determining concentration levels of one or more biomarker metabolites in the sample from the subject; and
diagnosing the subject as having depression or an increased risk of depression when the concentration levels of the one or more biomarker metabolites in the sample from the subject is different from (greater or less) than the concentration levels the one or more biomarker metabolites in a control sample.

54. The method of claim 53, further comprising:
initially treating the subject for depression by administering an effective amount sufficient to attenuate, reduce, or eliminate the symptoms of depression of one or more of antidepressants, cognitive behavior therapy, exercise, dietary supplements, prebiotics, probiotics, dietary changes, or an elimination diet for a period of time.

55. The method of claim 54, further comprising:
obtaining an additional sample from the subject and reevaluating the concentration level of the one or more biomarker metabolites in comparison to the subject's initial concentration levels of the one or more biomarker metabolites and the control concentration levels of the one or more biomarker metabolites;
evaluating the efficacy of the initial depression treatment; and
continuing the one or more initial depression treatments, administering an additional depression treatment, or administering one or more second depression treatments (switching the treatment regimen).

56. The method of claim 53 or 55, wherein the biomarker metabolite comprises one or more of:
Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine);
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine);
Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine;
Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline;
Glycerophospholipids (i.e., PC aa): PC aa C24:0; PC aa C26:0; PC aa C28:1; PC aa C30:0; PC aa C32:0; PC aa C32:1; PC aa C32:3; PC aa C34:1; PC aa C34:2; PC aa C34:3; PC aa C34:4; PC as C36:0; PC aa C36:1; PC aa C36:2; PC aa C36:3; PC aa C36:4; PC aa C36:5; PC aa C36:6; PC aa C38:0; PC aa C38:3; PC aa C38:4; PC as C38:5; PC as C38:6; PC as C40:1; PC as C40:2; PC as C40:3; PC as C40:4; PC aa C40:5; PC as C40:6; PC as C42:0; PC as C42:1; PC aa C42:2; PC aa C42:4; PC aa C42:5; or PC as C42:6;
Glycerophospholipids (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6;
Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1;
Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1;
short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs);
secondary bile acids (tauroursodeoxycholic acid (TDCA) and glycoursodeoxycholic acid (GUDCA));
3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline;
or combinations thereof.

57. The method of claim 53 or 55, wherein the sample from the subject is one or more of whole blood, serum, plasma, urine, saliva, or other body fluids.

58. The method of claim 53 or 55, wherein the control sample is from an untreated subject or a subject or population of subjects not experiencing depression or not at risk for depression.

59. The method of claim 53 or 55, wherein the biomarker metabolite concentration level increases compared to the initial sample or control.

60. The method of claim 53 or 55, wherein the biomarker metabolite concentration level decreases compared to the initial sample or control.

61. The method of claim 53 or 55, wherein two or more biomarker metabolite concentration levels covary and increase or covary and decrease (positive correlation) compared to the initial sample or control.

62. The method of claim 53 or 55, wherein two or more biomarker metabolite concentration levels vary dissimilarly (negative correlation) the initial sample or control.

63. The method of claim 53 or 55, wherein the depression is Major Depression Disorder (MDD), core depression (CD+), anxious depression (ANX+), neurovegetative symptoms of melancholia (NVSM+), or subclinical characteristics associated with depression.

64. The method of claim 54, wherein the period of time is at least: 4-weeks, 8-weeks, 12-weeks, 16-weeks, 32-weeks, 64-weeks, or greater than 64-weeks.

65. The method of claim 54, wherein the antidepressant is one or more of tranylcypromine, pheneizine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

66. The method of claim 54, wherein the antidepressant comprises escitalopram, citalopram, fluoxetine, sertraline, paroxetine, fluvoxamine, vilazodone, vortioxetine, or duloxetine.

67. The method of claim 54, wherein the antidepressant comprises escitalopram or citalopram.

68. The method of claim 53 or 55, wherein the depression and efficacy of treatment is evaluated using the Hamilton Depression Rating Scale (HRSD₁₇), the Quick Inventory of Depressive Symptomatology (QIDS), subscales thereof, or specific questions thereof.

69. The method of claim 68, wherein if the subject's depression score statistically significantly decreases from the initial score, the treatment is efficacious.

70. The method of claim 53, further comprising detecting at least one positively correlated biomarker metabolite, wherein detecting the at least one positively correlated biomarker metabolite is associated with a presence of at least one independent indicator of depression.

71. The method of claim 70, wherein the positively correlated biomarker metabolite is one or more of acylcarnitine C3, acylcarnitine C5, α-aminoadipic acid, sarcosine, serotonin (5HT), or 3-methoxy-4-hydroxyphenylglycol (MHPG), or combinations thereof.

72. The method of claim 70, wherein at least one independent indicator of depression comprises a HRSD₁₇ score of >18.

73. The method of claim 53, further comprising detecting at least one negatively correlated biomarker metabolite, wherein detecting the at least one negatively correlated biomarker metabolite is associated with an absence of at least one independent indicator of depression.

74. The method of claim 53, wherein the baseline detection of an increased level of at least one biomarker metabolite compared to a control comprising one or more of acylcarnitine C3, acylcarnitine C5, α-aminoadipic acid, sarcosine, serotonin or 3-methoxy-4-hydroxyphenylglycol (MHPG), or combinations thereof indicates the subject has at least one independent indicator of depression.

75. A method for predicting a depression subject's propensity of treatment success (depression remission) in response to an antidepressant, the method comprising:
obtaining a sample from the subject and evaluating the concentration level of the one or more biomarker metabolites in comparison to the control concentration levels of the one or more biomarker metabolites;
administering an effective amount sufficient to attenuate, reduce, or eliminate the symptoms of depression of one or more antidepressants, cognitive behaviorial therapy, or a combination thereof for a period of time;
obtaining an additional sample from the subject and evaluating the concentration level of the one or more biomarker metabolites in comparison to the subject's initial concentration levels of the one or more biomarker metabolites and the control concentration levels of the one or more biomarker metabolites;
continuing the one or more initial depression treatments, administering an additional depression treatment, or administering one or more second depression treatments (switching the treatment regimen).

76. The method of claim 75, wherein the biomarker metabolite comprises one or more of:
Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine);
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine);
Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine;
Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline;
Glycerophospholipids (i.e., PC aa): PC aa C24:0; PC aa C26:0; PC as C28:1; PC aa C30:0; PC aa C32:0; PC aa C32:1; PC aa C32:3; PC aa C34:1; PC aa C34:2; PC aa C34:3; PC aa C34:4; PC aa C36:0; PC aa C36:1; PC aa C36:2; PC aa C36:3; PC aa C36:4; PC aa C36:5; PC aa C36:6; PC aa C38:0; PC aa C38:3; PC aa C38:4; PC aa C38:5; PC aa C38:6; PC aa C40:1; PC aa C40:2; PC aa C40:3; PC as C40:4; PC as C40:5; PC aa C40:6; PC aa C42:0; PC as C42:1; PC as C42:2; PC aa C42:4; PC aa C42:5; or PC as C42:6;
Glycerophospholipids (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6;
Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1;
Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1;
short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs);
secondary bile acids (tauroursodeoxycholic acid (TDCA) and glycoursodeoxycholic acid (GUDCA));
3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), 5-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline;
or combinations thereof.

77. The method of claim 75, wherein the sample from the subject is one or more of whole blood, serum, plasma, urine, saliva, or other body fluids.

78. The method of claim 75, wherein the control sample is from an untreated subject or a subject or a population of subjects not experiencing depression or not at risk for depression.

79. The method of claim 75, wherein the depression is Major Depression Disorder (MDD), core depression (CD+), anxious depression (ANX+), neurovegetative symptoms of melancholia (NVSM+), or subclinical characteristics associated with depression.

80. The method of claim 75, wherein the period of time is at least: 4-weeks, 8-weeks, 12-weeks, 16-weeks, 32-weeks, 64-weeks, or greater than 64-weeks.

81. The method of claim 75, wherein the antidepressant comprises of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

82. The method of claim 75, wherein the antidepressant comprises escitalopram or citalopram.

83. The method of claim 75, wherein the depression and efficacy of treatment is evaluated using the Hamilton Depression Rating Scale (HRSD₁₇), the Quick Inventory of Depressive Symptomatology (QIDS), subscales thereof, or specific questions thereof.

84. The method of claim 75, wherein if the subject has greater baseline concentration levels of acylcarnitines C3, C5, α-aminoadipic acid, sarcosine, or serotonin in comparison to control concentration levels, and after at least 8-weeks of treatment these biomarker metabolites had concentrations greater than control levels, the subject has a propensity of antidepressant treatment success.

85. The method of claim 75, wherein if the subject has greater baseline concentration levels of acylcarnitines C3, C5, α-aminoadipic acid, sarcosine, or serotonin in comparison to control concentration levels, and the concentration levels of these biomarker metabolites remains greater than baseline after 8 weeks of antidepressant treatment, the subject has a propensity of antidepressant treatment success.

86. The method of claim 75, wherein if the subject has greater baseline concentration levels of one or more of 3-methoxy-4-hydroxyphenylglycol (MHPG) and serotonin (5HT) compared to control concentration levels and after at least 8-weeks of antidepressant treatment, the levels of MHPG and 5HT decreased compared to the baseline concentration levels, the subject has a propensity of antidepressant treatment success.

87. The method of claim 75, wherein if the subject has greater concentrations levels of one or more of PC as C34:1, PC aa C34:2, PC as C36:2 and PC aa C36:4 compared to baseline concentration levels after at least 8-weeks of antidepressant treatment, the subject has a propensity of antidepressant treatment success.

88. The method of claim 75, wherein if the subject has greater concentration levels after at least 8-weeks of antidepressant treatment of one or more of acylcarnitine (C5-M-DC), histidine, proline, kynurenine, and trans-4-hydroxyproline, PC aa C34:1, PC aa C34:2, PC aa C36:1, PC aa C36:2, PC aa C36:3; PC aa C36:4, PC aa C40:1, PC ae C34:2, PC ae C34:3, PC ae C36:1, PC ae C36:2, PC ae C36:3, PC ae C38:2, or PC ae C38:3, and the concentration levels of one or more of these biomarker metabolites are inversely associated with changes in the subject's depression score, the subject has a propensity of antidepressant treatment success.

89. A method for evaluating a depression subject's response to an antidepressant treatment, cognitive behavioral therapy or a combination thereof, the method comprising:
obtaining a sample from the subject and evaluating the concentration level of the one or more biomarker metabolites in comparison to the control concentration levels of the one or more biomarker metabolites;
administering an effective amount sufficient to attenuate, reduce, or eliminate the symptoms of depression of one or more antidepressants, cognitive behaviorial therapy, or a combination thereof for a period of time;
obtaining an additional sample from the subject and evaluating the concentration level of the one or more biomarker metabolites in comparison to the subject's initial concentration levels of the one or more biomarker metabolites and the control concentration levels of the one or more biomarker metabolites;
evaluating the efficacy of the initial depression treatment; and
continuing the one or more initial depression treatments, administering an additional depression treatment, or administering one or more second depression treatments (switching the treatment regimen).

90. The method of claim 89, wherein the biomarker metabolite comprises one or more of:
Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine);
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine);
Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine;
Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline;
Glycerophospholipids (i.e., PC aa): PC aa C24:0; PC aa C26:0; PC aa C28:1; PC aa C30:0; PC aa C32:0; PC aa C32:1; PC aa C32:3; PC aa C34:1; PC aa C34:2; PC aa C34:3; PC aa C34:4; PC as C36:0; PC aa C36:1; PC aa C36:2; PC aa C36:3; PC aa C36:4; PC aa C36:5; PC aa C36:6; PC aa C38:0; PC aa C38:3; PC aa C38:4; PC as C38:5; PC as C38:6; PC as C40:1; PC as C40:2; PC as C40:3; PC as C40:4; PC aa C40:5; PC as C40:6; PC as C42:0; PC as C42:1; PC aa C42:2; PC aa C42:4; PC aa C42:5; or PC as C42:6;
Glycerophospholipids (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6;
Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1;
Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1;
short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs);
secondary bile acids (tauroursodeoxycholic acid (TDCA) and glycoursodeoxycholic acid (GUDCA));
3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline;
or combinations thereof.

91. The method of claim 89, wherein the sample from the subject is one or more of whole blood, serum, plasma, urine, saliva, or other body fluids.

92. The method of claim 89, wherein the control sample is from an untreated subject or a subject or population of subjects not experiencing depression or not at risk for depression.

93. The method of claim 89, wherein the depression is Major Depression Disorder (MDD), core depression (CD+), anxious depression (ANX+), neurovegetative symptoms of melancholia (NVSM+), or subclinical characteristics associated with depression.

94. The method of claim 89, wherein the period of time is at least: 4-weeks, 8-weeks, 12-weeks, 16-weeks, 32-weeks, 64-weeks, or greater than 64-weeks.

95. The method of claim 89, wherein the antidepressant comprises one or more of tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

96. The method of claim 89, wherein the antidepressant comprises escitalopram or citalopram.

97. The method of claim 89, wherein the efficacy of the depression treatment is evaluated using the Hamilton Depression Rating Scale (HRSD₁₇), the Quick Inventory of Depressive Symptomatology (QIDS), subscales thereof, or specific questions thereof.

98. The method of claim 89, wherein after at least 8-weeks of antidepressant treatment, the subject has:
greater concentration levels compared to baseline concentration levels of acylcarnitine C3, C4 and C5; arginine, proline, methionine sulfoxide; phosphatidylcholines (PC as C36:1, C30:0, C42:2); phosphatidylcholine ethers (PC ae C34:3, C38:2, C36:3); and sphingomyelin SM C24:0; and
decreased concentration levels compared to baseline of acylcarnitines C8, C10, C12, C14:2, C16, C16:1, C18, C18:1, and C18:2; serotonin and sarcosine.

99. The method of claim 89, wherein after at least 8-weeks of antidepressant treatment, the subject has:
greater concentration levels compared to baseline levels of acylcarnitine indoles in TRP metabolism; 5-hydroxytryptophan (THTP), 5-hydroxyindoleacetic acid (5HIAA), indole-3-acetic acid (I3AA); homogentisic acid (HGA), vanillylmandelic acid, 4-hydroxyphenylacetic acid (4HPAC), 4-hydroxybenzoic acid (4HBAC), guanine (G), paraxanthine (PXAN), xanthosine (XANTH), salicylic acid (SA), the ratio of 4-hydroxyphenylacetic acid to tyrosine, the ratio of 5-hydroxyindoleacetic acid to serotonin, the ratio of uric acid to xanthosine, and the ratio of paraxanthine to xanthosine; and
decreased concentration levels of serotonin, 3-methoxy-4-hydroxyphenylethyleneglycol (MHPG), xanthosine, hypoxanthine, and the ratio of 3-methoxy-4-hydroxyphenylethyleneglycol to tyrosine.

100. The method of claim 89, wherein after at least 8-weeks of cognitive behavioral therapy, the subject has:
greater concentration levels compared to baseline levels of acylcarnitine (C0, C3, C4, and C5), α-aminoadipic acid, glutamate, and proline, isoleucine, aspartic acid, histidine, valine, tryptophan, tyrosine, phenylalanine, methionine, methionine-sulfoxide, and sarcosine; acylcarnitines (C10:2, C161-OH, C16-OH, C3:1, C3-OH, C4:1, C5:1, C5:1-DC, C5-OH (C3-DC-M), C6:1, C9); Phosphatidylcholines: (PCaa C24:0, C28:1, C30:0, C32:0, C32:1, C32:3, C34:1, C34:2, C34:3, C34:4, C36:1, C36:2, C36:3, C36:4, C36:5, C36:6, C38:0, C38:3, C38:4, C38:5, C38:6, C40:2, C40:4, C40:5, C40:6, C42:0, C42:1, C42:2, C42:4, C42:5, C42:6); (PC ae C30:2, C32:1, C32:2, C34:0, C34:1, C34:2, C34:3, C36:0, C36:1, C36:2, C36:3, C36:4, C36:5, C38:0, C38:2, C38:3, C38:4, C38:5, C38:6, C40:1, C40:2, C40:3, C40:4, C40:5, C40:6, C42:1, C42:2, C42:3, C42:4, C42:5, C44:3, C44:4, C44:5, C44:6); (LysoPC a C24:0, C26:0, C26:1, C28:0, C28:1); Sphingomyelins: SM (OH) C14:1, C22:1, C24:1, SM C16:0, C16:1, C18:0, C24:0, C24:1, C26:0, C26:1; and
decreases in the subject's depression score.

101. A method for differentiating a depression subject's depression phenotype, the method comprising:
identifying a subject experiencing depression or at risk of depression;
obtaining a sample from the subject and evaluating the concentration level of the one or more biomarker metabolites in comparison to the control concentration levels of the one or more biomarker metabolites;
administering an effective amount sufficient to attenuate, reduce, or eliminate the symptoms of depression of one or more antidepressants, cognitive behaviorial therapy, or a combination thereof for a period of time;
obtaining an additional sample from the subject and evaluating the concentration level of the one or more biomarker metabolites in comparison to the subject's initial concentration levels of the one or more biomarker metabolites and the control concentration levels of the one or more biomarker metabolites;
evaluating the phenotype and efficacy of the initial depression treatment; and
continuing the one or more initial depression treatments, administering an additional depression treatment, or administering one or more second depression treatments (switching the treatment regimen).

102. The method of claim 101, wherein the biomarker metabolite comprises one or more of:
Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine);
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine);
Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydoxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
Amino Acids: alanine; arginine; asparagine; aspartate; citrulline; glutamine; glutamate; glycine; histidine; isoleucine; lysine; methionine; omithine; phenylalanine; proline; serine; threonine; tryptophan; tyrosine; or valine;
Biogenic Amines: acetylomithine; asymmetric dimethylarginine; alpha-aminoadipic acid; camosine; creatinine; DOPA; dopamine; histamine; kynurenine; methionine sulfoxide; nitrotyrosine; phenylethylamine; putrescine; sarcosine; symmetric dimethylarginine; serotonin; spermidine; spermine; taurine; cis-4-hydroxyproline; or trans-4-hydroxyproline;
Glycerophospholipids (i.e., PC aa): PC aa C24:0; PC aa C26:0; PC aa C28:1; PC aa C30:0; PC aa C32:0; PC aa C32:1; PC aa C32:3; PC aa C34:1; PC aa C34:2; PC as C34:3; PC aa C34:4; PC aa C36:0; PC aa C36:1; PC as C36:2; PC aa C36:3; PC aa C36:4; PC as C36:5; PC as C36:6; PC as C38:0; PC as C38:3; PC aa C38:4; PC aa C38:5; PC aa C38:6; PC aa C40:1; PC aa C40:2; PC aa C40:3; PC aa C40:4; PC aa C40:5; PC aa C40:6; PC aa C42:0; PC aa C42:1; PC aa C42:2; PC aa C42:4; PC aa C42:5; or PC aa C42:6;
Glycerophospholipids (i.e., PC ae): PC ae C30:0; PC ae C30:1; PC ae C30:2; PC ae C32:1; PC ae C32:2; PC ae C34:0; PC ae C34:1; PC ae C34:2; PC ae C34:3; PC ae C36:0; PC ae C36:1; PC ae C36:2; PC ae C36:3; PC ae C36:4; PC ae C36:5; PC ae C38:0; PC ae C38:1; PC ae C38:2; PC ae C38:3; PC ae C38:4; PC ae C38:5; PC ae C38:6; PC ae C40:1; PC ae C40:2; PC ae C40:3; PC ae C40:4; PC ae C40:5; PC ae C40:6; PC ae C42:0; PC ae C42:1; PC ae C42:2; PC ae C42:3; PC ae C42:4; PC ae C42:5; PC ae C44:3; PC ae C44:4; PC ae C44:5; or PC ae C44:6;
Glycerophospholipids (i.e., Lyso PC): lysoPC a C14:0; lysoPC a C16:0; lysoPC a C16:1; lysoPC a C17:0; lysoPC a C18:0; lysoPC a C18:1; lysoPC a C18:2; lysoPC a C20:3; lysoPC a C20:4; lysoPC a C24:0; lysoPC a C26:0; lysoPC a C26:1; lysoPC a C28:0; or lysoPC a C28:1; or
Sphingolipids (i.e., SM): SM (OH) C14:1; SM C16:0; SM (OH) C16:1; SM C16:1; SM C18:0; SM C18:1; SM C20:2; SM (OH) C22:1; SM (OH) C22:2; SM C24:0; SM (OH) C24:1; SM C24:1; SM C26:0; or SM C26:1;
short-chain fatty acids (C3:0, C5:0); medium and long-chain fatty acids (C6:0, C9:0, C11:0, C10:0, C12:0, C13:0, C14:0, C15:0, C16:0, C17:0, C18:0, C20:3 (cis-8,11,14), C22:4 (cis-7,10,13,16); eicosapentaenoic acid (EPA, C20:5 (cis-5,8,11,14,17)); arachidonic acid (C20:4 (cis-5,8,11,14)); C22:4 (cis-7,10,13,16); C22:5 (cis-7,10,13,16,19); C20:5 (cis-5,8,11,14,17), C22:6 (cis-4,7,10,13,16,19), C22:5 (cis-7,10,13,16,19), C19:2 (cis-10,13); polyunsaturated fatty acids (PUFAs);
secondary bile acids (tauroursodeoxycholic acid (TDCA) and glycoursodeoxycholic acid (GUDCA));
3-hydroxykynurenine (3OHKY), 5-hydroxyindoleacetic acid (5HIAA), 5-hydroxytryptophan (5HTP), indole-3-acetic acid (I3AA), kynurenine (KYN), serotonin (5HT), 4-hydroxyphenylacetic acid (4HPAC), 4-hydroxyphenyllacetic acid (4HPLA), homogentisic acid (HGA), homovanillic acid (HVA), methoxy-hydroxyphenyl glycol (MHPG), vanillylmandelic acid (VMA), α-tocopherol (ATOCO), δ-tocopherol (DTOCO), γ-tocopherol (GTOCO), 4-hydroxybenzoic acid (4HBAC), guanine (G), guanosine (GR), hypoxanthine (HX), uric acid (URIC), xanthine (XAN), paraxanthine (PXAN), xanthosine (XANTH), salicylate (SA), α-methyltryptophan (AMTRP), indole-3-propionic acid (I3PA), hippuric acid, succinic acid, (±)-2-methylpentanoic acid; acetic acid, pelargonic acid, or theophylline;
or combinations thereof.

103. The method of claim 101, wherein the sample from the subject is one or more of whole blood, serum, plasma, urine, saliva, or other body fluids.

104. The method of claim 101, wherein the control sample is from an untreated subject or a subject or a population of subjects not experiencing depression or not at risk for depression.

105. The method of claim 101, wherein the depression is Major Depression Disorder (MDD), core depression (CD+), anxious depression (ANX+), neurovegetative symptoms of melancholia (NVSM+), or subclinical characteristics associated with depression.

106. The method of claim 101, wherein the period of time is at least: 4-weeks, 8-weeks, 12-weeks, 16-weeks, 32-weeks, 64-weeks, or greater than 64-weeks.

107. The method of claim 101, wherein the antidepressant comprises one or more tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram.

108. The method of claim 101, wherein the antidepressant comprises escitalopram or citalopram.

109. The method of claim 101, wherein the type of depression (phenotype) and efficacy of the depression treatment is evaluated using the Hamilton Depression Rating Scale (HRSD₁₇), the Quick Inventory of Depressive Symptomatology (QIDS), subscales thereof, or specific questions thereof.

110. The method of claim 101, wherein the baseline detection of an decreased concentration level of at least one biomarker metabolite compared to a control comprising one or more of acylcarnitines C0, C3, C18m, C5:1, C5-DC/C6-OH, C16-OH, or combinations thereof indicates the subject has at least one independent indicator of core depression (CD+).

111. The method of claim 101, wherein the baseline detection of an decreased concentration level of at least one biomarker metabolite compared to a control comprising one or more of acylcarnitines C5-DC/C6-OH and C16-OH, or combinations thereof and higher concentration levels of acylcarnitine C10 compared to a control, indicates the subject has at least one independent indicator of neurovegetative symptoms of melancholia (NVSM+).

112. The method of claim 101, wherein after at least 8-weeks of antidepressant treatment, subjects having core depression (CD+), had greater concentration levels of acylcarnitines C5-DC/C6-OH, C5-M-DC, C6, C14:1, C16, C16-OH, C16:1, and C18:1-OH compared with concentration levels of subjects having neurovegetative symptoms of melancholia (NVSM+).

113. The method of claim 101, wherein after at least 8-weeks of antidepressant treatment, subjects having core depression (CD+), had lower concentration levels of acylcarnitines C2, C3 and C6 compared with anxious depression (ANX+) subjects.

114. The method of claim 101, wherein after at least 8-weeks of antidepressant treatment, subjects having neurovegetative symptoms of melancholia (NVSM+), had lower concentration levels of acylcarnitines C0, C2, C3, C5-DC/C6-OH, C5-M-DC, C6, C10, C14:1, C16, C16-OH, C16:1 and C18:1-OH compared with anxious depression (ANX+) subjects.

115. The method of claim 101, wherein after at least 8-weeks of antidepressant treatment, subjects having core depression (CD+), had increased concentration levels of acylcarnitines C0, C3, C4, C5-M-DC and C5:1 compared with baseline concentration levels of these biomarker metabolites.

116. The method of claim 101, wherein after at least 8-weeks of antidepressant treatment, subjects having core depression (CD+) or anxious depression (ANX+), had increased concentration levels of acylcarnitines C0, C3, and C4 compared with baseline concentration levels of these biomarker metabolites.

117. The method of claim 101, wherein after at least 8-weeks of antidepressant treatment, subjects having core depression (CD+) or neurovegetative symptoms of melancholia (NVSM+), had decreased concentration levels of acylcarnitines C8, C10, and C12 compared with baseline concentration levels of these biomarker metabolites.

118. The method of claim 101, wherein after at least 8-weeks of antidepressant treatment, subjects having core depression (CD+) or neurovegetative symptoms of melancholia (NVSM+), had decreased concentration levels of acylcarnitines C14:1, C14:2, C16, C16-OH, C16:1, C18, C18:1, C18:1-OH, C18:2 compared with baseline concentration levels of these biomarker metabolites.

119. The method of claim 101, wherein after at least 8-weeks of antidepressant treatment, subjects having core depression (CD+), neurovegetative symptoms of melancholia (NVSM+) or anxious depression (ANX+), had decreased concentration levels of acylcarnitines, C5-OH, C16:1, C18:1, and C18:2, compared with baseline concentration levels of these biomarker metabolites.
